(19) United States
(12) Patent Application Publication
(10) Pub. No.: US 2020/0316231 A1
(43) Pub. Date: Oct. 8, 2020

(54) Compositions And Methods For Imaging Immune Cells

(71) Applicant: The Trustees of the University of Pennsylvania, Philadephia, PA (US)

(72) Inventor: Mark A. Sellmyer, Philadelphia, PA (US)

(21) Appl. No.: 16/870,484

(22) Filed: May 8, 2020

Related U.S. Application Data
(63) Continuation-in-part of application No. 16/511,052, filed on Jul. 15, 2019.
Application No. 16/511,052 is a division of application No. 15/572,632, filed on Nov. 8, 2017, now Pat. No. 10,398,790.
Application No. 15/572,632 is a continuation-in-part of application No. 16/123,797, filed on Sep. 6, 2018.
(60) Provisional application No. 62/845,707, filed on May 9, 2019.
(60) Provisional application No. 62/159,327, filed on May 10, 2015.
(60) Provisional application No. 62/554,699, filed on Sep. 6, 2017.

(51) Int. Cl. A61K 51/04; A61K 31/395; A61K 31/495; A61K 49/00; C07D 239/49; C07D 403/12; A61K 47/54; A61K 31/505

(52) U.S. Cl. CPC A61K 51/0459; A61K 31/395; A61K 31/495; A61K 49/0021; A61K 49/0052; A61K 51/0453; C07B 2200/05; C07D 239/49; C07D 403/12; A61K 47/547; A61K 31/505; Y02A 50/473; Y02A 50/481; A61K 51/0497

(57) ABSTRACT

The present disclosure provides immunes cells comprising a radiolabeled tracer useful in imaging tests such as positron emission topography (PET)/computed tomography (CT) scans. The present disclosure further includes engineered cells comprising a chimeric antigen receptor (CAR) further comprising a nucleic acid molecule comprising a ligand binding domain capable of binding to radiolabeled tracer. This disclosure also includes methods for assessing the efficacy or toxicity of an adoptive cell therapy in a subject, methods for detecting the quantity of engineered T cells in a subject, methods for monitoring an immunotherapy treatment in a subject and methods of imaging engineered T cells in a subject. In some embodiments, the radiolabeled tracer is [¹⁸F]fluoropropyl-trimethoprim ([¹⁸F]FPTMP).

### CROSS REFERENCE TO RELATED APPLICATIONS

This application claims the benefit of the priority of U.S. Provisional Patent Application No. 62/845,707, filed May 9, 2019, which is incorporated herein by reference in its entirety.

This application is also a continuation-in-part of U.S. patent application Ser. No. 16/511,052, filed Jul. 15, 2019, which is a divisional application of U.S. patent application Ser. No. 15/572,632, filed Nov. 8, 2017, now U.S. Pat. No. 10,398,790, which is the U.S. national stage of International Patent Application No. PCT/US2016/031600, filed May 10, 2016, which claims the benefit of the priority of U.S. Provisional Patent Application No. 62/159,327, filed May 10, 2015, each of which is incorporated herein by reference in its entirety.

This application is also a continuation-in-part of U.S. patent application Ser. No. 16/123,797, filed Sep. 6, 2018, which claims the benefit of the priority of U.S. Provisional Patent Application No. 62/554,699, filed Sep. 6, 2017, each of which is incorporated herein by reference in its entirety.

### SEQUENCE LISTING

The Sequence Listing for this application is labeled “103241.006526 Sequence Listing.txt,” which was created on May 1, 2020 and is 32 Kilobytes. The entire content is incorporated herein by reference in its entirety.

### TECHNICAL FIELD

This invention relates to the field of imaging immune cells using radiolabeled tracers.

### BACKGROUND

Given the increasing importance of cell-based therapies, there is a great need to develop techniques that allow for rapid characterization of new strategies and molecular targets. Imaging is particularly important for clinical management of patients with cancer, and molecular imaging, most notably with the radiotracers FDG and DOTATATE, has played an important role in the assessment of target expression and treatment response. Similar position emission tomography (PET) radiotracers to monitor a cell-based therapy with respect to proper trafficking to the target or development of off-target toxicity, have been slow in developing in part because of the high barrier to entry of such nuclear molecular imaging technologies coupled with the various challenges of the clinical implementation for gene therapy. Several groups have worked on developing new cell-based PET reporter genes, with strong preclinical benchmarking of strategies that use for example, herpes simplex virus thymidine kinase (HSV-tk), human neuroendocrine receptor (hNET), and prostate specific membrane antigen (PSMA). In these studies, hNET paired with meta-¹⁸F-fluorobenzylguanidine [¹⁸F]MFBG was extrapolated to be capable of identifying approximately 30,000-45,000 engineered cells. Still, only HSV-tk has been applied clinically, and its limitations include immunogenicity of the enzyme as well as background uptake at the site of a tumor. In general, reporter genes require the addition of a receptor, transporter, or enzyme to the host cells, they have the potential to reduce the viability or efficacy of those cells, which limits their clinical application if they are only being employed for imaging.

Cell-based therapeutics have considerable promise across a variety of fields, from regenerative medicine to cancer therapies. However, reliable human imaging of the distribution and trafficking of genetically engineered cells such as chimeric antigen receptor (CAR) T cells remains a challenge.

There is a need in the art for improved methods to track engineered cells, including immune cells used for cell-based therapy and adoptive immunotherapy.

### SUMMARY OF THE INVENTION

The present disclosure includes compositions and methods of using small molecules for positron emission tomography (PET) imaging of immunes cells.

In one aspect, provided herein are engineered cells comprising a chimeric antigen receptor (CAR) and further comprising a nucleic acid molecule comprising a ligand binding domain capable of binding to a radiolabeled tracer. In one embodiment, the cells are T cells.

In another aspect, methods of assessing the efficacy or toxicity of an adoptive cell therapy in a subject are provided. The methods comprise: (a) administering to the subject an engineered T cell comprising a chimeric antigen receptor (CAR) and a nucleic acid molecule comprising a ligand binding domain; (b) administering to the subject a radiolabeled tracer capable of binding to the ligand binding domain; (c) detecting the amount of radiolabeled tracer bound by imaging; and, (d) assessing the efficacy or toxicity of the adoptive cell therapy in the subject.

In another aspect, methods of detecting the quantity of engineered T cells in a subject are provided. The methods comprise (a) administering to the subject an engineered T cell comprising a chimeric antigen receptor (CAR) and a nucleic acid molecule comprising a ligand binding domain; (b) administering to the subject a radiolabeled tracer capable of binding to the ligand binding domain; and, (c) imaging the amount of radiolabeled tracer bound thereby detecting the quantity of engineered T cells in the subject.

In a further aspect, methods of monitoring an immunotherapy treatment in a subject are also provided. The methods comprise (a) administering to the subject an engineered T cell comprising a chimeric antigen receptor (CAR) and a nucleic acid molecule comprising a ligand binding domain; (b) administering to the subject a radiolabeled tracer capable of binding to the ligand binding domain; and, (c) detecting the level of radiolabeled tracer bound by imaging as a measure of the immunotherapy treatment. In some embodiments, the imaging is performed by positron emission tomography (PET), computed tomography (CT) or bioluminescence (BL).

In yet another aspect, methods of imaging engineered T cells in a subject are provided. The methods comprise (a) administering to the subject an engineered T cell comprising a chimeric antigen receptor (CAR) and a nucleic acid molecule comprising a ligand binding domain; (b) administering to the subject a radiolabeled tracer capable of binding to the ligand binding domain; and, (c) detecting the radiolabeled tracer by imaging using positron emission tomography (PET) or computerized tomography (CT).

In one embodiment, the ligand binding domain is E. coli dihydrofolate reductase (eDHFR). In one embodiment, the radiolabeled tracer is [¹⁸F]fluoropropyl-trimethoprim ([¹⁸F]FPTMP). In one embodiment, the engineered T cell(s) is/are autologous to the subject. In another embodiment, the engineered T cell(s) is/are allogenic to the subject. In another embodiment, the subject is a mammal. In yet another embodiment, the mammal is a human.

Other aspects and embodiments of the invention will be readily apparent from the following detailed description of the invention.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific compositions, methods, devices, and systems disclosed. In addition, the drawings are not necessarily drawn to scale.

FIG. 1 is a diagram of TMP as a PET radiotracer coupled with Ec DFHR reporter gene in mammalian cells.

FIG. 2 is a dose-response and competition dot blot showing various concentrations of recombinant Ec DHFR spotted on to a nitrocellulose membrane.

FIG. 3 are fluorescent microscopy images of HEK293 and HCT 116 cells and virally transduced YFP-DHFR cells assessed for yellow fluorescent protein transgene expression.

FIG. 4 is a bar graph of counts per minute (cpm) of HEK 293 control cells or DHFR cells incubated with [¹¹C]TMP with and without competing cold TMP (10 μM) or methotrexate (MTX) (10 μM). The competing compounds were added at 10 μM. Error bars represent standard deviation (n=3).

FIG. 5 is a bar graph of counts per minute (cpm) of HCT 116 control cells or DHFR cells incubated with [¹¹C]TMP with and without competing cold TMP (10 μM). Error bars represent standard deviation (n=3). Mammalian DHFR inhibitor methotrexate (10 μM) was added and showed little effect on specificity of TMP binding.

FIG. 6 is a bar graph of counts per minute (cpm) of bacteria (E. coli) incubated with [¹¹C]TMP after being assayed for uptake with a gamma counter. Error bars represent standard deviation (n=3).

FIG. 7 is a bar graph illustrating the fold induction from of the data of FIG. 7. It is the signal in live bacterial group over heat killed or live bacterial with cold TMP competition.

FIG. 8 is a plot of cpm vs. colony forming units (cfu/mL) showing that [¹¹C]TMP shows a greater binding/signal ratio in the presence of more live bacteria.

FIG. 9 is a bar graph illustrating the fold induction of live bacteria (Staph, Pseudomonas, and E. coli) over heat killed bacteria.

FIG. 10 the immunoblot probing for YFP of YFP-DHFR fusion proteins. The expected fusion protein molecular weight is 45 kDa. GADPH provided a loading control.

FIGS. 11A/B are line and bar graphs, respectively, showing the quantification of SUV (signal/dose/g) from small animal micro PET/CT scans of DHFR or control tumors with [¹¹C]TMP where HCT116 tumors were xenografted subcutaneously (10 million cells) to the shoulders of nude mice. The tumors were grown for 10 days and imaged using small animal PET followed by CT. Error bars represent standard deviation (n=3). B) Fold change of SUV (DHFR signal divided by control signal) with and without oral TMP block.

FIGS. 12A/B are bar graphs showing the in vivo biodistribution of [¹¹C]TMP in the absence and presence of an oral TMP block. The Biodistribution studies were completed 90 minutes after injection of [¹¹C]TMP. Dissected tissues were analyzed with a gamma counter. Error bars represent standard deviation (n=3).

FIG. 13 is a bar graph showing the sensitivity of detection of transduced DHFR reporter cells. HEK293 DFHR cells were injected subcutaneously in matrigel (150 μL) at concentrations of 3×10⁶, 3×10⁵, and 3×10⁴ cells 24 h prior to radiotracer administration and PET imaging. The matrigel was harvested and measured on a gamma counter. Error bars represent the SEM (n=3) and there was statistical significance of 3×10⁶ and 3×10⁵ cells (P<0.05 Student T, two tailed).

FIG. 14 is an autoradiograph of an explanted GI tract from the stomach to the rectum of a mouse.

FIGS. 15A/B are thin layer films and radio-chromatographs showing the spot size and relative counts from mouse urine compared to parent [¹¹C]TMP.

FIG. 16 is a graph illustrating the percent of radiosignal in the bladder while under anesthesia (no micturition) as assessed by measuring the signal in the bladder divided by the total counts in the animal over time (n=1).

FIG. 17A/B are bar graphs showing the results of [¹⁸F]FPTMP uptake/specificity studies in HCT116 DHFR cells (FIG. 17A) and HCT116 (FIG. 17B) cells.

FIG. 18 is a line graph showing a Bₘₐₓ of 2870±106 fmol/mg and K_{d} of 0.465±0.07 nM in HCT and HCT116 DHFR cells.

FIG. 19 is a line graph showing [¹⁸F]FPTMP uptake after serial dilutions of 293 (•) and 293 DHFR (▪) cells.

FIG. 20A illustrates the quantification of in vivo uptake data in an experiment performed similarly to FIG. 11 but with [¹⁸F]FPTMP. Error bars represent standard deviation (n=3). FIG. 20B is an expanded section of the 0 to 2.5 y-axis over 50 minutes.

FIGS. 21A/B are bar graphs showing the normalized signal in tissue and biodistribution of [¹⁸F]FPTMP and quantification of tumor to muscle ratios for the control and DHFR. Error bars represent standard deviation (n=3).

FIGS. 22A/B are bar graphs showing the % uptake of [¹⁸F]FPTMP in the absence and presence of cold TMP for heat-killed S. aureus and E. coli bacteria after 15 minutes (FIG. 22A) and 3 hours (FIG. 22B).

FIG. 23 is a schematic and image of a mouse injected with turpentine (left leg), live bacteria (right leg), heat killed bacteria (right shoulder) and mouse breast cancer cells (left shoulder) prior to imaging.

FIGS. 24A-H are coronal (FIGS. 24A-C) and axial (FIGS. 24D-F) images of a live mouse after injection of [¹⁸F]FPTMP and next day injection and imaging of [¹⁸F]FDG. The data shows [¹⁸F]FPTMP uptake only in live bacteria, but in turpentine inflammation. The data shows [¹⁸F]FDG uptake only in both live bacteria and turpentine inflammation. FIGS. 24G-24H are bar graphs showing quantification of the levels of uptake as seen in FIG. 24, in infection versus inflammation for FPTMP (FIG. 24G) and FDG (FIG. 24H).

FIGS. 25A-F are coronal (FIGS. 25A-C) and axial (FIGS. 275-F) images of a live mouse after injection of [¹⁸F]FPTMP and next day injection and imaging of [¹⁸F]FDG showing the absence of [¹⁸F]FPTMP uptake in tumor cells and the presence of [¹⁸F]FDG in tumor cells.

FIGS. 26A-26C are series of drawings of the structure of eDHFR PET reporter protein, trimethoprim, [¹⁸F]FPTMP and reporter plasmids. FIG. 26A) Bacterial DHFR complexed with NADPH and TMP (10.2210/pdb3FRE/pdb).²⁹ FIG. 26B) Structures of trimethoprim (TMP) and [¹⁸F]fluoropropyl-trimethoprim, [¹⁸F]FPTMP. FIG. 26C) Triple reporter plasmid in pELPS with eDHFR (PET reporter gene) fused to yellow fluorescent protein (YFP) and a T2A cleavage site followed by Renilla luciferase, and CAR plasmid pELPS with GD2-scFv-CD8 hinge 4-1BB-CD3z and a 2A cleavage site followed by mCherry selection marker.

FIGS. 27A-27E are series of images and graphs depicting [¹⁸F]FPTMP uptake in eDHFR cells in vitro and in vivo. FIG. 27A) Time course of in vitro uptake of eDHFR transduced HCT116 cells with methotrexate (MTX, 10 μM) and unlabeled TMP (10 μM) as blocking agents (n=4). FIG. 27B) Similar to (FIG. 27A) but with untransduced, control HCT116 cells. FIG. 27C) Scheme of location of eDHFR HCT116 and control tumors. FIG. 27D) Representative small animal PET/CT images in axial and coronal planes. FIG. 27E) Time course quantification of eDHFR tumor uptake compared to control from FIG. 27D (n=3). Error bars represent the SD.

FIGS. 28A-28C are series of graphs showing in vitro bioluminescence imaging (BLI) and [¹⁸F]FPTMP uptake of DYR-transduced primary human T cells. FIG. 28A) Primary T-cells were transduced with pELPS DHFR-YFP-T2A-Renilla (DYR) and sorted on YFP expression. 1×10⁵ DYR T cells or NTD T cells were incubated with 1.5 uM coelenterazine in a 96-well plate and subjected to BLI. Mean total flux is shown for each group (n=3). FIG. 28B) 1×10⁶ DYR or NTD T cells were incubated with 2×10⁶ counts per minute (CPM) of [¹⁸F]FPTMP for 30 minutes prior to washings with PBS. Uptake as a percentage of injected dose (% ID) was measured by gamma counting and normalized per million cells (n=3). FIG. 28C) Primary human T cells that had been activated with anti-CD3/CD28 antibody-coated beads were co-transduced with DYR lentivirus as well as lentivirus encoding the GD2-E101K-4-1BB CAR containing a mCherry fluorescent protein separated by a T2A site. The population of double positive (DP) T cells was isolated by flow cytometric cell sorting. (Collected DP population shown in red). T cells within the YFP gate (blue) were collected to serve as CAR-negative control DYR T cells. Error bars represent the SEM.

FIGS. 29A-29F are series of graphs and images showing in vivo CAR T cell trafficking. FIG. 29A) NSG immunodeficient mice were xenografted in the subcutaneous shoulder regions with GD2+ tumors (143b human osteosarcoma, right shoulder) and GD2-tumors (HCT116 human colon cancer cells, left shoulder), 10 million cells per tumor. The tumors were grown for 14 days when mice were injected with 1×10⁶ DYR-CAR T cells or control DYR T cells via tail vein. The mice were imaged on day 7 and 13, first with BLI after coelenterazine (ctz) injection via tail vein and then with PET/CT after [¹⁸F]FPTMP injection (˜100 μCi via tail vein). For quantification, regions of interest were drawn around the entire tumors, and the signal maximum in the tumors was divided by the signal maximum from the heart/mediastinal blood pool signal, thereby yielding a target to background ratio. FIG. 29B) [¹⁸F]FPTMP target to background ratio in the spleen was increased on day 7 in several mice treated with DYR-CAR T cells, which decreased by day 13. Mice treated with control DYR T cells showed no significant splenic signal over background. FIG. 29C) Mouse 2 (M2, plus sign) demonstrated BLI and PET signal from the spleen at day 7 that decreased by day 13 (red arrowheads and red arrows). FIG. 29D) Quantification of PET signal over background at the site of GD2+ 143b tumor at days 7 and 13 in the mice receiving DYR-CAR T cells (left panel) and mice receiving DYR control T cells (right panel). FIG. 29E) BLI of mouse 4 (M4, plus sign), showing T cells present in the spleen at day 7 and then concentrated at the site GD2+ tumor on day 13. Focal areas of PET signal in the GD2+ tumor on PET/CT images are highlighted with red arrows. FIG. 29F) Neither DYR-CAR nor DYR control T cells showed focal areas of significant uptake in HCT116 tumors that do not express the GD2 epitope. DYR-CAR Mouse 3 (M3, asterisk) did show some signal above background on ex vivo [¹⁸F]FPTMP autoradiography, that was supported by anti-human CD8 IHC.

FIGS. 30A-30B are series of images depicting DYR-CAR M4 GD2− tumor, and GD2+ tumor autoradiography and immunohistochemistry (IHC) for rad-path correlation. FIG. 30A) Gross specimen of GD2− HCT116 tumor paired with [¹⁸F]FPTMP ex vivo autoradiography (acquired same day as day 13 PET imaging), and overlay image. FIG. 30 B) Gross specimen of GD2+ 143b tumor as in (FIG. 30A). The areas of autoradiography signal correlate with the areas containing positive anti-human CD8 IHC staining cells. Automated detection of tissue (yellow outlines) and DAB positive cells (red marks) was performed using QuPath software.

FIG. 31 is a table providing an evaluation of eDHFR and Control 116 [¹⁸F]FPTMP uptake with competitive inhibitors. Values displayed are in % Input/100 μg Protein (n=4).

FIGS. 32A-32B are series of graphs depicting an ex vivo biodistribution of tissues including eDHFR and Control HCT116 tumors. Mice were sacrificed at the completion of the PET/CT imaging session. FIG. 32A) Uptake in percent injected dose per gram (% ID/g) was assay with a gamma counter (n=3). FIG. 32B) Ratio of tumor uptake to muscle (n=3). Error bars represent the standard deviation.

FIG. 33 is a series of graphs illustrating HCT116 cell GD2 staining with flow cytometry. HCT116 (left panel) and known GD2⁺ positive control SY5Y cells (right panel) were stained with APC isotype control or APC-anti-GD2 and analyzed by flow cytometry.

FIG. 34 is an image of DYR-CAR M4 spleen. IHC was performed on DYR-CART M4 spleens for CD8 (see FIGS. 29A-29F showing persistent PET signal from the M4 spleen on day 13). CD8 positive CAR T cells are present in the splenic periphery (20×).

FIGS. 35A-35D are series of images depicting BLI, PET/CT, autoradiography and IHC of DYR-CAR M3 tumors. FIG. 35A) BLI shows splenic signal on D7 that decreased by D13 (arrow). New foci appear on D13 in the tumors. The areas in the 143b tumor are highlighted by the red box while the areas in the HCT116 tumor are indicated with the arrowhead. B) PET/CT shows foci of uptake (red boxes) in the peripheral and medial areas of the 143b (GD2+) tumor and low level of generalized uptake in the HCT116 tumor. FIG. 35C) Autoradiography of a random section of the 143b tumor shows scattered areas of uptake and one area of radiosignal that correlates with CD8 IHC (red boxes). FIG. 35D) An area of peripheral uptake on autoradiography correlates with the area of CD8 IHC (large blue box). The small blue boxes are 20× zoom of the areas on the thumbnail image.

FIGS. 36A-36C are series of images showing BLI, autoradiography and IHC of Control M1 tumors. FIG. 36A) BLI shows foci of signal overlying the HCT116 tumor (GD2−). FIG. 36B) Autoradiography of the 143b tumor shows scattered areas of uptake overlying the bone of the upper arm. There were no CD8 positive T cells on IHC. FIG. 36C) The HCT116 tumor did not show any specific signal on auto-radiography and there were no CD8 positive T cells on IHC.

FIGS. 37A-37B are HLA peptide motif search results listing the predicted half-time of dissociation to HLA class one molecules (SEQ ID NOs: 4-45).

FIG. 38 is an image depicting an estimation of cellular density where PET signal can be determined. CD8 DYR-CAR T-cells were counted on a medium power field (10×). There are 200 cells counted (Software: Image J). The result for calculating the volume in cu millimeters (mm³) of a rectangular box shape, with a length of 1.65 millimeters (mm), a width (thickness) of 10 micrometers (μm) and a height of 1.1 millimeters (mm) is 0.01815 mm³. Thus, per mm³ the number of cells needed for micro PET/CT detection is approximately (1/0.01815)*200=(55.1)*200=11,000 cells per mm³.

FIGS. 39A-39B are series of diagrams illustrating iCasp9 pathway and the ligand-induced degradation (LID) system. FIG. 39A: iCasp9 is based on human caspase 9, in which the recruitment domain of the caspase has been replaced by F36V-FKBP. This allows the caspase pathway to be activated by the small molecule AP1903, which is a dimer of Shield-1 that causes apoptosis via dimerization of the F36V-FKBP/caspase 9 fusion protein. FIG. 39B: The LID system is based on the addition of a degradation sequence (degron) to the C terminus of F36V-FKBP, which is then fused to a protein of interest. In the absence of Shield-1 the degron is bound to FKBP and the protein is stable. However, when Shield-1 is present, it binds tightly to FKBP, displaces the degron, and induces rapid degradation of the LID domain and the fused protein of interest.

FIG. 40 is a graph depicting the chemical structures of Shield-1 and AP1903.

FIGS. 41A-41B are series of graphs and images showing cell uptake with [¹¹C]TMP. FIG. 41A: HEK293 cell uptake studies with [¹¹C]TMP. FIG. 41B: Small animal PET/CT imaging of DHFR+ and DHFR− tumors with [¹¹C]TMP.

FIGS. 42A-42B are series of chemical structures illustrating the routes of synthesis of Shield-1 precursor. FIG. 42A: First route, compound A was synthesized as a mixture of enantiomers. FIG. 42B: Second route, stereoselective synthesis of compound A, which was then coupled to the secondary amine (synthesized as in the 1^{st} route). No HPLC separation of diastereomers was needed.

FIGS. 43A-43C are series of chemical structures and graphs depicting the properties of [¹¹C]Shld1. FIG. 43A: Synthesis of [¹¹C]Shld1. FIG. 43B: HPLC analysis of [¹¹C]Shld1 compared with cold Shield-1 from Cheminpharma, LLC. FIG. 43C: Luminescence of HCT116 cells stably expressing L106P-tsLuc following treatment with cold Shield-1 (3 nM to 10 uM) and incubation with luciferin.

FIGS. 44A-44B are series of histograms showing [¹¹C]Shld1 uptake in HEK293 cells (FIG. 44A) and in HCT116 cells (FIG. 44B) after 40 min. HEK293 and HCT116 cells transduced with F36V-FKBP take up C-11 Shield-1 well at 40 minutes. The uptake can be blocked by a large excess of cold FK506 (10 and WT cells do not take up C-11 Shield-1, thus the amount of nonspecific binding is low.

FIG. 45 is a series of images illustrating HEK 293-F36V-FKBP-YFP cells. The nucleus was stained with DAPI.

FIG. 46 is a histogram depicting [¹¹C]Shield-1 biodistribution study. 50 μCi of [¹¹C]-Shield-1 was injected via the tail vein, and mice were sacrificed at 2 min, 25 min, and 45 min following injection of radiotracer (n=4 mice per time point).

FIG. 47 is a graph demonstrating caspase activation following treatment of CAR T cells with AP20187. The inducible caspase 9 was incorporated into CART cells directed against desmoglein-3, and was activated by treatment with varying concentrations of AP20187, a chemical inducer of dimerization which is an analog of AP1903.

FIGS. 48A-48B is a series of chemical structures illustrating the synthesis of [¹⁸F]-fluoroethyl-Shield-1 (FIG. 48A) and other [¹⁸F] labeled derivatives of Shield-1 (FIG. 48B).

FIG. 49 is a series of diagrams illustrating the generation of pBMN-eDHFR-Casp9.

FIG. 50 is a western blot depicting the eDHFR-iCasp9 fusion protein in various cells: HEK293, HCT116 and MB231.

FIG. 51 is a series of images depicting the labeling of DHFR-icasp9 cells with Ligandlink (fluorescein label with TMP): WT cells do not bind to fluorescent TMP, but MB231 cells transduced with DHFR-iCasp9 do.

FIG. 52 is a series of images depicting the labeling of DHFR-icasp9 cells with Ligandlink (fluorescein label with TMP): WT cells do not bind to fluorescent TMP, but HEK293 cells transduced with DHFR-iCasp9 do.

FIG. 53 is a series of images depicting the labeling of DHFR-icasp9 cells with Ligandlink (fluorescein label with TMP): WT cells do not bind to fluorescent TMP, but HCT116 cells transduced with DHFR-iCasp9 do.

FIG. 54 is a graph illustrating a [¹⁸F]-TMP cell uptake study. MB231 cells transduced with DHFR-iCasp9 take up [¹⁸F]-TMP rapidly, and the uptake slightly increases over time. The uptake can be blocked by a large excess of cold TMP, thus the nonspecific binding is low.

FIG. 55 is a histogram illustrating a [¹⁸F]-TMP cell uptake study. MB231 cells transduced with DHFR-iCasp9 take up [¹⁸F]-TMP, with similar uptake at 30 min and 120 min. The uptake can be blocked by a large excess of cold TMP, and WT cells do not take up [¹⁸F]-TMP, thus the amount of nonspecific binding is low.

FIG. 56 is a graph illustrating a [¹⁸F]-TMP cell uptake study. HCT116 cells transduced with DHFR-iCasp9 take up [¹⁸F]-TMP rapidly, and the uptake is stable over time. The uptake can be blocked by a large excess of cold TMP, thus the amount of nonspecific binding is low.

FIG. 57 is a histogram illustrating a [¹⁸F]-TMP cell uptake study. HCT116 cells transduced with DHFR-iCasp9 take up [¹⁸F]-TMP rapidly, with similar uptake at 30 min and 120 min. The uptake can be blocked by a large excess of cold TMP, and WT cells do not take up [¹⁸F]-TMP, thus the amount of nonspecific binding is low.

FIG. 58 is a histogram illustrating a [¹⁸F]-TMP cell uptake study. HEK293 cells transduced with DHFR-iCasp9 take up [¹⁸F]-TMP well at 30 min. The uptake can be blocked by a large excess of cold TMP, and WT cells do not take up [¹⁸F]-TMP, thus the amount of nonspecific binding is low.

FIG. 59 is a series of chemical structures illustrating the synthesis of fluoropropyl-Shield-1 (FP-Shield-1) from the Shield-1 precursor.

FIG. 60 is a graph depicting the luminescence of HCT116 cells stably expressing L106P-tsLuc following treatment with increasing concentrations of either cold Shield-1 (from Cheminpharma, LLC) or FP-Shield-1, and incubation with luciferin. This graph demonstrates that the affinity of FP-Shield-1 for F36V-FKBP is similar to commercially available Shield-1.

FIGS. 61A-61F are a series of graphs depicting the % viability of MDA-MB231 cells that have been transduced with different DHFR-iCasp9 constructs, and treated with a variety of Bis-TMP compounds at increasing concentrations. The linker between DHFR and iCasp9 was modified to be 5, 6, 9, 15, or 18 amino acids in length. The linker between the TMP molecules in the Bis-TMP compounds was modified to be 6, 8, 10, 16, 21, or 27 atoms in length. Wild-type MDA-MB231 cells were also evaluated as a control. Most combinations did not produce any cell killing; only DHFR-iCasp9 linkers of 15 or 18 amino acids, and Bis-TMP linkers of 21 and 27 atoms demonstrated cell killing.

FIGS. 62A-62B are a series of graphs depicting the % viability of MDA-MB231 cells that have been transduced with DHFR-15-iCasp9 or DHFR-18-iCasp9 (with 15 or 18 amino acid linkers, respectively) and treated with Bis-TMP-21 or Bis-TMP-27 (with 21 or 27 atoms linkers, respectively) at increasing concentrations. The graphs illustrate potent activation of the DHFR-iCasp9 suicide gene at low nM concentrations of Bis-TMP. The DHFR-18-iCasp9 suicide gene and Bis-TMP-27 were the most effective combination, killing ˜70% of cells with an IC₅₀ of ˜5 nM.

FIGS. 63A-63B are a series of chemical structures depicting all of the Trimethoprim (TMP) compounds of the present disclosure. Bis-TMP=bis-trimethoprim and Tris-TMP=tris-trimethoprim.

### DETAILED DESCRIPTION OF THE INVENTION

Trimethoprim (TMP) is a small molecule antibiotic routinely used in the clinic that has high affinity and specificity for the E. coli dihydrofolate reductase enzyme (Ec DHFR), a bacterial protein involved in DNA synthesis that is highly genetically conserved across many bacterial species. The Ec DHFR protein is a small, 159 residue, 18 kDa essential enzyme involved in DNA and amino acid synthesis in all living organisms that is often used in biochemical studies and protein engineering tools.

To date, there is no single agent that provides a facile and repeatable imaging tool for long term tracking of engineered cells, an increasingly important mode of therapy for cancer and other diseases. It is additionally apparent that multiple genetic reporter genes capable of imaging and tracking multiple types of cells are needed as the complexity of cell therapy advances.

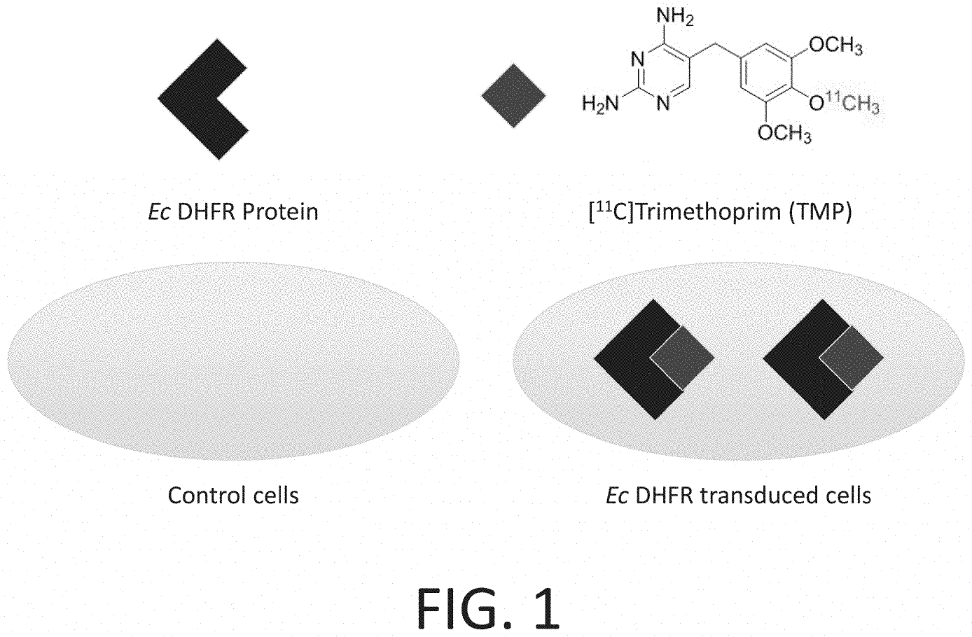

The TMP radiolabeled compounds described have high specificity for DHFR at very low concentrations. FIG. 1 is a diagram of TMP as a PET radiotracer coupled with Ec DFHR reporter gene. The compounds described herein permit efficient diagnosis thereby suggesting significant downstream cost differences. Specifically, the ability of the compounds described herein, due to their efficacy in identifying locations of infection, may lead to the use of fewer and less antibiotics, appropriate care for patients, reduced lengths of hospital stays, and avoidance of unnecessary surgery and biopsy. In one embodiment, if there is increased signal after compound administration from an area of clinical concern when compared to appropriate control tissue, the increased signal correlates which a highly suggestion of bacterial infection.

The compounds discussed herein have a wide range of uses. In one embodiment, the compounds are capable of imaging bacteria, both commensal and infectious. In another embodiment, the compounds may be used as a positron emission tomography (PET) reporter probe for imaging transgenic cells. The binding and retention of TMP by Ec DHFR will allow for clearance of non-bound probe providing high contrast imaging of bacteria or transgenic cells carrying Ec DHFR in whole animals or humans. The ability to longitudinally and non-invasively monitor basic bacterial infections such as pneumonia, osteomyelitis, cystic fibrosis superinfection or transgene expression in engineered cells used for cancer immunotherapy would be a powerful, groundbreaking advance beyond current standard PET imaging technologies and could revolutionize the diagnostic imaging armamentarium for many related clinical settings.

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range. In some embodiments, the measurable is an amount or a temporal duration. In some embodiments, the term “about” refers to variations of ±20%, ±10%, ±5%, ±1%, or ±0.1% from the specified value, as such variations are appropriate to perform the disclosed methods. When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

The term “antigen” or “Ag” as used herein is defined as a molecule that provokes an immune response. This immune response may involve either antibody production, or the activation of specific immunologically-competent cells, or both. The skilled artisan will understand that any macromolecule, including, e.g, proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

“Allogeneic” refers to any material derived from a different animal of the same species.

“Xenogeneic” refers to any material derived from an animal of a different species.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have specificity to a selected target, for example a B cell surface receptor. CARs may also comprise an intracellular activation domain, a transmembrane domain and an extracellular domain comprising a tumor associated antigen binding region. In some aspects, CARs comprise an extracellular domain comprising an anti-B cell binding domain fused to CD3-zeta transmembrane and intracellular domain.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

The term “CRISPR/CAS,” “clustered regularly interspaced short palindromic repeats system,” or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via RNA-guided DNA cleavage.

In the type II CRISPR/Cas system, short segments of foreign DNA, termed “spacers” are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

To direct Cas9 to cleave sequences of interest, crRNA-tracrRNA fusion transcripts, hereafter referred to as “guide RNAs” or “gRNAs” may be designed, from human U6 polymerase III promoter. CRISPR/CAS mediated genome editing and regulation, highlighted its transformative potential for basic science, cellular engineering and therapeutics.

The term “CRISPRi” refers to a CRISPR system for sequence specific gene repression or inhibition of gene expression, such as at the transcriptional level.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

The term “FK506 Binding Protein (FKBP)” refers to a family of proteins that have prolyl isomerase activity and are related to the cyclophilins in function, though not in amino acid sequence. FKBPs have been identified in many eukaryotes from yeast to humans and function as protein folding chaperones for proteins containing proline residues. FKBPs belong to the immunophilin family. In some embodiments, the FKBP is F36V-FKBP. In some embodiments, a dimerized ligand capable of binding FKBP is useful for the methods of this invention. In some embodiments, the dimerized ligand capable of binding FKBP refers to a compound represented by the structure:

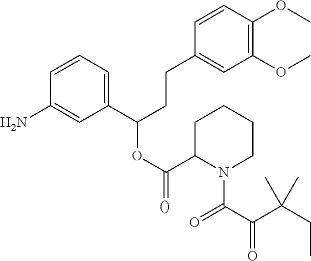

or any derivative or analog thereof.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are about 50% homologous; if about 90% of the positions, are matched or homologous, the two sequences are about 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. In some embodiments, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “imaging” or “tracking” as used herein refers to any method of scanning the body of a subject using techniques such as positron emission tomography (PET), computed tomography (CT), single photon emission computed tomography (SPECT), bioluminescence imaging (BLI) among others.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

When “an immunologically effective amount,” “an autoimmune disease-inhibiting effective amount,” or “therapeutic amount” is indicated, the precise amount of the compositions to be administered can be determined by a physician or researcher with consideration of individual differences in age, weight, tumor size, extent of infection or metastasis, and condition of the patient (subject).

As used herein, an “instructional material” includes a publication, a recording, a diagram, or any other medium of expression which can be used to communicate the usefulness of the compositions and methods herein. The instructional material of the kit may, for example, be affixed to a container which contains the nucleic acid, peptide, and/or composition or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “limited toxicity” as used herein, refers to the peptides, polynucleotides, cells and/or antibodies manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The terms “patient” or “subject” as used herein refer to a mammalian animal. In one embodiment, the patient or subject is a human. In another embodiment, the patient or subject is a veterinary or farm animal, a domestic animal or pet, or animal normally used for clinical research. In still a further embodiment, the subject or patient has cancer. The subject or patient has either been recognized as having or at risk of having cancer.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “suicide gene” as used herein refers to a suicide or apoptosis-inducing gene operably linked to a promoter, which may be constitutive or inducible. Examples of a suicide gene include, but are not limited to, a herpes simplex virus thymidine kinase (HSV-TK), the cytoplasmic domain of Fas, a caspase such as caspase-8 or caspase-9, cytosine deaminase, E1A, FHIT, and other known suicide or apoptosis-inducing genes. The suicide gene comprises a ligand binding domain and a suicide domain.

The term “suicide gene product” as used herein refers to the expression product of the suicide gene.

The term “suicide domain” as used herein means that portion of the suicide gene that when activated induces DNA cleavage, generally leading to apoptosis of a cell in which the suicide domain resides.

A “ligand binding domain” as used herein means a domain that binds a ligand, for example a radiolabeled tracer or a small molecule suicide switch.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In some aspects, the disclosure provides engineered cells comprising a chimeric antigen receptor (CAR) and further comprising a nucleic acid molecule comprising a ligand binding domain capable of binding to a radiolabeled tracer. In one embodiment, the cells are T cells.

In further aspects, the disclosure provides uses of cells known in the art that can be genetically modified. In some embodiments, the genetically engineering cell is a T-cell, NK-cell, macrophage, B-cell, stem cell, hematopoietic stem cell, mesenchymal stem cell, neuroprogenitor cell, induced pluripotent cell, or any combination thereof.

Other aspects include methods for assessing the efficacy or toxicity of an adoptive cell therapy in a subject, methods for detecting the quantity of engineered T cells in a subject, methods for monitoring an immunotherapy treatment in a subject and methods of imaging engineered T cells in a subject.

In some embodiments, the imaging is performed via Positron Emission Topography (PET). In other embodiments, the efficacy or toxicity of the adoptive cell therapy in the subject is assessed and continuation, modification or termination of the therapy is recommended.

In some embodiments, the radiolabeled tracer is [¹⁸F]fluoropropyl-trimethoprim ([¹⁸F]FPTMP). In other embodiments, the radiolabeled tracer can be a monomer or a dimer selected from the group consisting of [¹¹C]-Trimethoprim ([¹¹C]-TMP) and [¹⁸F]-Trimethoprim ([¹⁸F]-TMP), or any radiolabeled derivative known in the art capable of binding to dihydrofolate reductase (DHFR). Various TMP based radiolabeled tracers are described in US Patent Application Publication No. US-2016/031600, incorporated herein by reference in its entirety.

In some embodiments, the radiolabeled tracer is administered to the subject at very low concentrations, so its occupies about 1 to about 5% of the available target receptors. In some embodiments, the administered radiolabeled tracer does not have a measurable physiological effect on the subject.

The radiolabeled tracer compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining target protein concentration and/or substrate receptor occupancy. Isotopically-labeled compounds are prepared by any suitable method or by processes using an appropriate isotopically-labeled reagent in place of the non-labeled reagent otherwise employed.

One aspect of this invention includes an engineered cell comprising a chimeric antigen receptor (CAR) and further comprising a nucleic acid comprising a suicide gene comprising a ligand binding domain and a suicide domain wherein the ligand binding domain is capable of binding to radiolabeled tracer or a small molecule suicide switch.

The invention contemplates use of any cell known in the art that can be genetically modified. In some embodiments, the genetically engineering cell is a T-cell, NK-cell, macrophage, B-cell, stem cell, hematopoietic stem cell, mesenchymal stem cell, neuroprogenitor cell, induced pluripotent cell, or any combination thereof.

Other aspects of this invention includes methods for inducing apoptosis of an engineered cell, methods for assessing the efficacy or toxicity of an adoptive cell therapy in a subject, methods for detecting the quantity of engineered T cells in a subject, methods for monitoring an immunotherapy treatment in a subject and methods of imaging engineered T cells in a subject.

In some embodiments, the methods of the invention comprise administering to the subject an engineered T cell comprising a chimeric antigen receptor (CAR) and a nucleic acid molecule comprising a suicide gene that comprises a ligand binding domain and a suicide domain, administering to the subject a radiolabeled tracer capable of binding to the ligand binding domain; and, detecting the amount of radiolabeled tracer bound by imaging.

In other embodiments, the efficacy or toxicity of the adoptive cell therapy in the subject is assessed and continuation, modification or termination of the therapy is recommended. In some embodiments, when adoptive cell therapy is assessed as toxic the radiolabeled tracer is replaced by a cold (non radiolabeled) dimerized ligand capable of binding to the ligand binding domain and activating the suicide domain thereby inducing cell death of the engineered T cells.

In some embodiments, the radiolabeled tracer is a radiolabeled monomeric or a dimeric compound. In some embodiments, the radiolabeled tracer is a radiolabeled monomer. In some embodiments, the radiolabeled tracer is selected from the group consisting of [¹¹C]-Shield-1, [¹⁸F]-Shield-1, or any radiolabeled derivative known in the art capable of binding to FKBP (such as F36V-FKBP). In other embodiments, the radiolabeled tracer is a monomer or a dimer selected from the group consisting of [¹¹C]-Trimethoprim ([¹¹C]-TMP) and [¹⁸F]-Trimethoprim ([¹⁸F]-TMP), or any radiolabeled derivative known in the art capable of binding to dihydrofolate reductase (DHFR).

In some embodiments, the radiolabeled tracer is administered to the subject at very low concentrations, so its occupies 1-5% of the available target receptors. In some embodiments, the administered radiolabeled tracer does not have a measurable physiological effect on the subject.

Another aspect of this invention includes a chemical inducer of dimerization (CID) wherein the CID is a dimerized ligand capable of binding to the ligand binding domain of the suicide gene and activating it thereby inducing cell death.

In certain embodiments, the CID is a Bis-Trimethoprim (Bis-TMP) of Formula (I), or a salt, solvate, enantiomer, diastereoisomer or tautomer thereof:

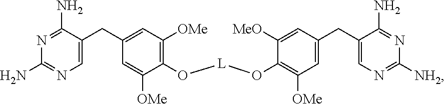

wherein L is a chemical linker having a length of about 1 to about 50 atoms.

In certain embodiments, L is an alkylene, heteroalkylene, alkenylene, heteroalkenylene, alkynylene, or heteroalkynylene. In other embodiments L has a backbone consisting of about 31 atoms. In other embodiments L has a backbone consisting of 25 atoms. In yet other embodiments L has a backbone consisting of 19 atoms. In yet other embodiments L has a backbone consisting of 14 atoms.

In certain embodiments, L is a linker selected from the group consisting of: —(CH₂)ₖ—;

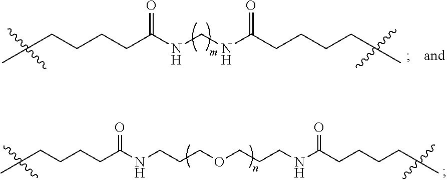

wherein k is an integer from 1-50, m is an integer from 1-38 and n is an integer from 1-11.

In certain embodiments, k is selected from the group consisting of 31, 25, 19 and 14. In other embodiments, m is selected from the group consisting of 19, 13, 7 and 2. In yet other embodiments, n is selected from the group consisting of 1, 3 and 5.

In certain embodiments, the Bis-TMP of Formula (I) is a compound selected from the group consisting of:

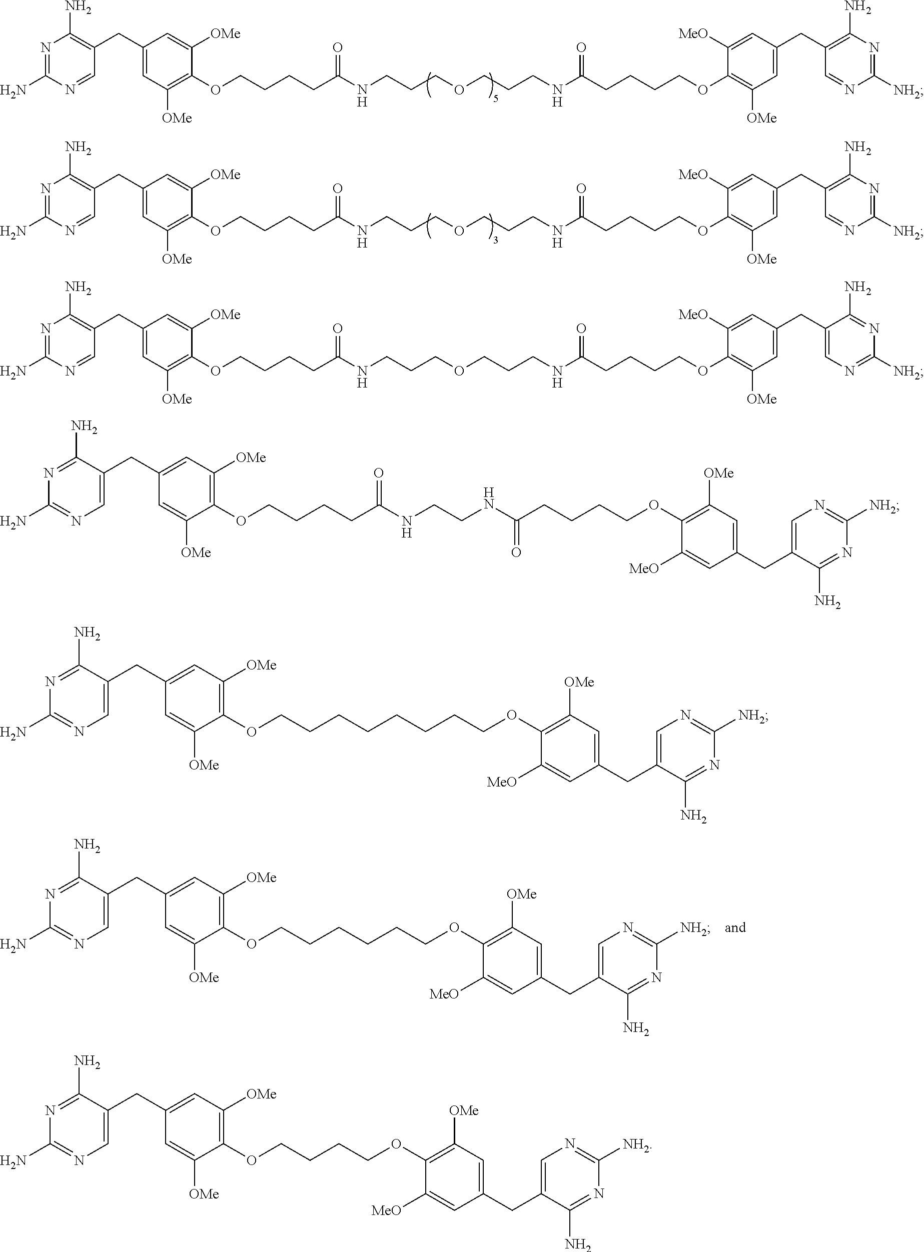

Another aspect of this invention includes an engineered cell comprising achimeric antigen receptor (CAR) and further comprising a ligand-induced degradation (LID) system and a ligand wherein the ligand comprises a radiolabeled tracer. In some embodiments, a degradation sequence (degron) is fused to the C terminus of F36V-FKBP.

In one embodiment, the ligand of F36V-FKBP is a compound of formula (II).

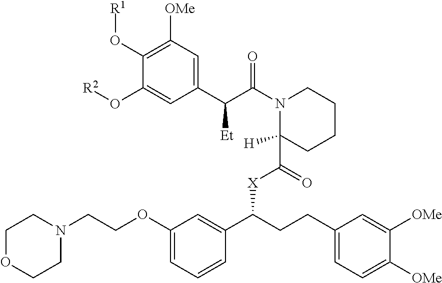

wherein

X is NH or O;

a) R¹ is CH₂Y, CH₂CH₂Y, or CH₂CH₂CH₂Y and R² is CH₃; or

b) R² is CH₂Y, CH₂CH₂Y, or CH₂CH₂CH₂Y and R¹ is CH₃; and

Y is F or ¹⁸F.

In other embodiments, the radiolabeled tracer is selected from the group consisting of [¹¹C]-Shield-1; [¹⁸F]-Shield-1. In some embodiments, the radiolabeled tracer is replaced by a cold non radiolabeled ligand selected from the group consisting of Shield-1, AP1903 and AP20187, wherein the cold non radiolabeled ligand is capable of degrading the CAR.

Suicide Gene:

Some of the potential side effects of CAR T cells can be overcome by the co-expressing a suicide gene in the CAR T cell. In the present invention, a radiolabeled tracer is added to the engineered CAR T cells of the invention and these cells are then tracked in vivo, monitored and selectively removed in case toxicity arises in the subject or after targeting B cells for depletion when treating a disease or condition.

The methods of the invention include use of an isolated nucleic acid comprising a suicide gene. Examples of suicide genes include, but are not limited to, herpes simplex virus thymidine kinase (HSV-TK), the cytoplasmic domain of Fas, a caspase such as caspase-8 or caspase-9, cytosine deaminase, E1A, FHIT, and other known suicide or apoptosis-inducing genes (Straathof et al., 2005, Blood 105:4247-4254; Cohen et al., 1999, Leuk. Lymphoma 34:473-480; Thomis et al., 2001, Blood 97:1249-1257; Tey et al., 2007, Biol. Blood Marrow Transplant 13:913-924; and Di Stasi et al., 2011, N. Engl. J. Med. 365:1673-1683).

The suicide gene may be operably linked to a promoter, such as an inducible promoter sequence. Examples of inducible promoters include, but are not limited to, a heat shock promoter, a tetracycline-regulated promoter, a steroid-regulated promoter, a metal-regulated promoter, an estrogen receptor-regulated promoter, and others known in the art. In one aspect, the invention includes an isolated nucleic acid sequence comprising a nucleic acid sequence comprising a suicide gene and a nucleic acid encoding a chimeric antigen receptor. In another aspect, the invention includes an isolated nucleic acid sequence comprising a suicide gene and a nucleic acid encoding a chimeric antigen receptor.

In one embodiment, the suicide gene is under the control of an inducible promoter.

In yet another embodiment the suicide domain is an inducible caspase 9 (iCasp9) domain.

In some embodiments, the suicide gene is in an expression vector. In an exemplary embodiment, the present invention includes a vector comprising a nucleic acid sequence comprising a suicide gene. The expression vector may also include other genes, such as a chimeric antigen receptor and/or CRISPR system disclosed elsewhere herein.

The invention also includes a cell comprising the suicide gene. In an exemplary aspect, the present invention includes a modified cell comprising a nucleic acid comprising a suicide gene and a nucleic acid encoding a chimeric antigen receptor.

In one embodiment, the CAR modified T cell comprises nucleic acids encoding a suicide gene as a separate nucleic acid sequence from the CAR construct. For example, HSV-TK, iCasp9, the cytoplasmic domain of Fas, or a caspase can be incorporated into genetically engineered T cells separate from the CAR construct. In another embodiment, the CAR modified T cell comprises a suicide gene in the same construct as the nucleic acids encoding the CAR. In this embodiment, the nucleic acid comprising the suicide gene may be upstream or downstream of the nucleic acid encoding the CAR.

In one embodiment, expression of the suicide gene is activated in the cell by contacting the cell with an inducing agent administered to the cell or to a subject comprising the cell. The inducing agent then activates an inducible promoter to express the suicide gene. In such an embodiment, the inducing agent is administered to the subject to induce expression of the suicide gene.

In one embodiment, the suicide binding domain comprises a ligand binding domain selected from the group consisting of FKBP, F36V-FKBP, E. coli dihydrofolate reductase (eDHFR). eDHFR is a bacterial protein involved in DNA synthesis that is highly genetically conserved across many bacterial species. The eDHFR protein is a small, 159 residue, 18 kDa essential enzyme involved in DNA and amino acid synthesis in all living organisms that is often used in biochemical studies and protein engineering tools. In one embodiment, iCasp9 domain comprises eDHFR ligand binding domain (eDHFR-iCasp9). In another embodiment, eDHFR-iCasp9 further comprises a linker consisting of 15 or 18 amino acids in length. In some embodiments, the linker comprises Ser-Gly-Gly-Gly-Ser (SEQ ID NO: 1) amino acids motifs.

In another embodiment, the ligand is a monomer or a dimer radiolabeled tracer. In some embodiments, the radiolabeled tracer is a monomer that does not activate the suicide gene.

In one embodiment, the radiolabeled tracer is selected from the group consisting of [¹¹C]-Shield-1; [¹⁸F]-Shield-1; [¹¹C]-Trimethoprim ([¹¹C]-TMP) and [¹⁸F]-Trimethoprim ([¹⁸F]-TMP). Shield-1 is a monomer with high affinity for F36V-FKP domain. TMP is a small molecule antibiotic routinely used in the clinic that has high affinity and specificity for the eDHFR (see for instance US patent application US2016/0272618 incorporated herein by reference in its entirety). As a monomer, TMP also does not activate the suicide gene iCasp9. Various TMP based radiolabeled tracers are described in US Patent application US2016/031600, incorporated herein by reference in its entirety. The radiolabeled tracer compounds listed herein are stabilizing compounds and do not trigger the activation of the suicide gene unless dimerized.

In some embodiments, a suicide gene product that is expressed from the suicide gene is activated by an activating agent, such as a dimerization agent. For example, the dimerization agents, such as AP1903 (rimiducid), AP20187 or any derivative or analog known in the art, promote dimerization and activation of caspase-9 molecules. In other instances, the dimerization agent is a chemical inducer of dimerization (CID) such as BIS-Trimethoprim (Bis-TMP), or any derivative or analog thereof promotes dimerization and activation of DHFR caspase-9 molecules. In some embodiments where constitutive expression of the suicide gene is initially desired, expression of the suicide gene may be turned off in the cell by contacting the cell with an inhibiting agent administered to the cell or to a mammal comprising the cell. The inhibiting agent selectively turns off expression. For example, caspase-9 is constitutively expressed in the cell and the addition of an inhibiting agent represses expression or activation of caspase-9. In one embodiment, the inhibiting agent is administered to the subject to repress expression of the suicide gene.

In some embodiments where constitutive expression of the suicide gene is initially desired, activation of the suicide gene product may be repressed in the cell by contacting the cell with an inhibiting agent, such as a solubilizing agent, administered to the cell or to a mammal comprising the cell. The inhibiting agent represses activation of the suicide gene product, such as by preventing dimerization of the caspase-9 molecules. In one embodiment, the solubilizing agent is administered to the subject to repress activation of the suicide gene product.

In some aspects, the suicide gene is not immunogenic to the cell comprising the suicide gene or host harboring the suicide gene. Although thymidine kinase (TK) may be employed, it can be immunogenic. Alternatively, examples of suicide genes that are not immunogenic to the host include caspase-9, caspase-8, and cytosine deaminase.

In yet another embodiment, suicide gene expression is linked in tandem to dimerization domains, which cause aggregation and degradation of the transcript, preventing cell-surface expression and hence function of the suicide gene.

In some embodiments, a solubilizing agent can be useful. Solubilization of the dimerization domains with a solubilizing agent, administered to the cell or to a mammal comprising the cell, prevents aggregation and allows the construct to egress through the secretory system.

Chimeric Antigen Receptor (CAR)

Described herein are engineered cells with a CAR and a suicide gene and a ligand comprising a radiolabeled tracer. Also described are nucleic acids encoding a CAR or an engineered cell (e.g. T cell) comprising a CAR, wherein the CAR includes an antigen binding domain, a transmembrane domain and an intracellular domain.

One or more domains or a fragment of a domain of the CAR may be human. In one embodiment, the disclosure provides a fully human CAR. The nucleic acid sequences coding for the desired domains can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than as a cloned molecule.

Examples of CARs are described in U.S. Pat. Nos. 8,911,993, 8,906,682, 8,975,071, 8,916,381, 9,102,760, 9,101,584, and 9,102,761, all of which are incorporated herein by reference in their entireties.

Antigen Binding Domain

In one embodiment, the CAR comprises an antigen binding domain that binds to a B cell. Cell surface markers selectively found on B cells may act as an antigen that binds to the antigen binding domain of the CAR.

The anti-B cell antigen binding domain can include any domain that binds to the B cell and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof, such as a single chain variable fragment (scFv).

The antigen binding domain may bind one or more B cell antigens, such as, but not limited to, any surface marker selectively found on a B cell, such as a pro-B cell, pre-B cell, immature B cell, mature B cell, memory B cell, and plasma cell. In one embodiment, the antigen binding domain binds at least one B cell antigen, such as CD19, BCMA, and any combination thereof. In another embodiment, the antigen binding domain binds at least one B cell antigen, such as CD20, CD21, CD27, CD38, CD138, and any combination thereof.

In some instances, it is beneficial for the antigen binding domain to be derived from the same species in which the CAR will ultimately be used in. For example, for use in humans, it may be beneficial for the antigen binding domain of the CAR to comprise a human antibody, humanized antibody as described elsewhere herein, or a fragment thereof.

It is also beneficial that the antigen binding domain is operably linked to another domain of the CAR, such as the transmembrane domain or the intracellular domain, both described elsewhere herein, for expression in the cell. In one embodiment, a nucleic acid encoding the antigen binding domain is operably linked to a nucleic acid encoding a transmembrane domain and a nucleic acid encoding an intracellular domain.

In addition to B cell antigen binding domains, the antigen binding domain can be any antigen binding domain suitable for introduction into a CAR construct, where binding of the antigen binding domain to its cognate binding partner has a beneficial effect on a subject.

Transmembrane Domain

With respect to the transmembrane domain, the CAR can be designed to comprise a transmembrane domain that connects the antigen binding domain of the CAR to the intracellular domain. In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some instances, a variety of human hinges can be employed as well including the human Ig (immunoglobulin) hinge.

In one embodiment, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

Intracellular Domain

The intracellular domain or otherwise the cytoplasmic domain of the CAR is responsible for activation of the cell in which the CAR is expressed. The term “intracellular domain” is thus meant to include any portion of the intracellular domain sufficient to transduce the activation signal. In one embodiment, the intracellular domain includes a domain responsible for an effector function. The term “effector function” refers to a specialized function of a cell. Effector function of a T cell, for example, may be cytolytic activity or helper activity including the secretion of cytokines.

In one embodiment, the intracellular domain of the CAR includes a domain responsible for signal activation and/or transduction. The intracellular domain may transmit signal activation via protein-protein interactions, biochemical changes or other response to alter the cell's metabolism, shape, gene expression, or other cellular response to activation of the chimeric intracellular signaling molecule.

Examples of an intracellular domain for use herein include, but are not limited to, the cytoplasmic portion of the T cell receptor (TCR) and any co-stimulatory molecule that acts in concert to initiate signal transduction following antigen receptor engagement, as well as any derivative or variant of these elements and any synthetic sequence that has the same functional capability. In one embodiment, the intracellular domain of the CAR comprises dual signaling domains. The dual signaling domains may include a fragment or domain from any of the molecules described herein.

Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rb), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combination thereof.

In one embodiment, the intracellular domain of the CAR includes any portion of a co-stimulatory molecule, such as at least one signaling domain from CD3, CD27, CD28, CD83, CD86, CD127, 4-1BB, 4-1BBL, PD1, PD1L, T cell receptor (TCR), any derivative or variant thereof, any synthetic sequence thereof that has the same functional capability, and any combination thereof.

Between the antigen binding domain and the transmembrane domain of the CAR, or between the intracellular domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In one embodiment, the spacer domain may comprise up to about 300 amino acids, preferably about 10 to about 100 amino acids and most preferably about 25 to about 50 amino acids. In another embodiment, a short oligo- or polypeptide linker, preferably between about 2 and about 10 amino acids in length may form the linkage between the transmembrane domain and the intracellular domain of the CAR. An example of a linker includes a glycine-serine doublet.

In some embodiments, the CAR further comprises a signal peptide.

In some embodiments, the CAR further comprises a dimerization domain, such a dimerization domain from FKBP, F36V-FKBP, F36M-FKBP, E. coli dihydrofolate reductase (eDHFR) or similar molecule. In such an embodiment, the presence of CAR molecules on the surface of the modified T cell is prevented by spontaneous aggregation of the CAR molecules in the cytoplasm or other internal location in the cell.

Between the antigen binding domain and the transmembrane domain of the CAR, or between the intracellular domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In one embodiment, the spacer domain may comprise up to 300 amino acids, preferably 10 to 100 amino acids and most preferably 25 to 50 amino acids. In another embodiment, a short oligo- or polypeptide linker, preferably between 2 and 10 amino acids in length may form the linkage between the transmembrane domain and the intracellular domain of the CAR. An example of a linker includes a glycine-serine doublet.

In some embodiments, the CAR further comprises a signal peptide.

In some embodiments, the CAR further comprises a dimerization domain, such a dimerization domain from FKBP, F36V-FKBP, F36M-FKBP, E. coli dihydrofolate reductase (eDHFR) or similar molecule. In such an embodiment, the presence of CAR molecules on the surface of the modified T cell is prevented by spontaneous aggregation of the CAR molecules in the cytoplasm or other internal location in the cell.

CRISPR/Cas

The present invention further includes modifying the T cell described herein by deleting the endogenous T cell receptor (TCR) and/or major histocompatibility complex (MHC) molecules with genome editing technology to reduce or prevent the transmission of stimulatory signals through the endogenous TCR/MHC complex.

The CRISPR/Cas system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/CAS system can thereby be engineered to cleave virtually any DNA sequence by redesigning the gRNA in cell lines (such as 293T cells), primary cells, and CAR T cells. The CRISPR/CAS system can simultaneously target multiple genomic loci by co-expressing a single CAS9 protein with two or more gRNAs, making this system uniquely suited for multiple gene editing or synergistic activation of target genes.

One example of a CRISPR/Cas system used to inhibit gene expression, CRISPRi, is described in U.S. Publication No.: 2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In one embodiment, the CRISPR system comprises an expression vector, such as, but not limited to, an pAd5F35-CRISPR vector. In one embodiment, the modified T cell described herein is further modified by introducing a Cas expression vector and a guide nucleic acid sequence specific for a gene into the modified T cell. In another embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

The guide nucleic acid sequence is specific for a gene and targets that gene for Cas endonuclease-induced double strand breaks. The sequence of the guide nucleic acid sequence may be within a loci of the gene. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

The guide nucleic acid sequence may be specific for a T cell receptor (TCR) chain (such as an alpha, beta, gamma and/or delta chain), a major histocompatibility complex protein (such as a HLA class I molecule and/or HLA class II molecule), and any combination thereof.

The guide nucleic acid sequence includes a RNA sequence, a DNA sequence, a combination thereof (a RNA-DNA combination sequence), or a sequence with synthetic nucleotides. The guide nucleic acid sequence can be a single molecule or a double molecule. In one embodiment, the guide nucleic acid sequence comprises a single guide RNA.

In some embodiments, a T cell is modified to express a CAR and the CAR T cell is further modified to delete endogenous TCR or MHC molecules, such as before administration to a subject. In one embodiment, the CAR modified T cell described herein is further modified by deleting a gene selected from the group consisting of a T cell receptor (TCR) chain, a major histocompatibility complex protein, and any combination thereof. In another embodiment, the T cell is modified before administration to the subject in need thereof.

In some embodiments, a T cell is modified to express a CAR, administered to a subject, and then further modified in vivo to delete endogenous TCR or MHC molecules, such as through inducing targeted gene deletion. In one embodiment, the modified T cell described herein is modified by inducing a CRISPR/Cas system to minimize native reactivity of the modified T cell or host reactivity to the modified T cell. In some embodiments, inducing the Cas expression vector comprises exposing the modified T cell to an agent that activates an inducible promoter in the Cas expression vector. In such an embodiment, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In some embodiments, a T cell is modified to delete endogenous TCR or MHC molecules prior to modification to express the CAR. In some embodiments, the modified T cell is further modified by deleting TCR or MHC molecules prior to inducing expression of the suicide gene.

Introduction of Nucleic Acids

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001)).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

RNA

In one embodiment, the nucleic acids introduced into the T cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a chimeric membrane protein. By way of example, the template encodes an antibody, a fragment of an antibody or a portion of an antibody. By way of another example, the template comprises an extracellular domain comprising a single chain variable domain of an antibody, such as anti-CD3, and an intracellular domain of a co-stimulatory molecule. In one embodiment, the template for the RNA chimeric membrane protein encodes a chimeric membrane protein comprising an extracellular domain comprising an antigen binding domain derived from an antibody to a co-stimulatory molecule, and an intracellular domain derived from a portion of an intracellular domain of CD28 and 4-1BB.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a capon the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA is electroporated into the cells, such as in vitro transcribed RNA.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors may be utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in US Patent Application Publication Nos. 2004/0014645, 2005/0052630, 2005/0070841, 2004/0059285, and 2004/0092907A. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in, e.g., U.S. Pat. Nos. 6,567,694; 6,516,223, 5,993,434, 6,181,964, 6,241,701, and 6,233,482; electroporation may also be used for transfection of cells in vitro as described e.g. in U.S. Patent Application Publication No. 2007/0128708. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

Sources of T Cells

Prior to expansion, a source of T cells is obtained from a subject. Non-limiting examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, umbilical cord, and tumors. In certain embodiments, any number of T cell lines available in the art, may be used. In certain embodiments, T cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. In some embodiments, the method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of about 2 billion cells/ml is used. In one embodiment, a concentration of about 1 billion cells/ml is used. In a further embodiment, greater than about 100 million cells/ml is used. In a further embodiment, a concentration of cells of about 10, 15, 20, 25, 30, 35, 40, 45, or 50 million cells/ml is used. In yet another embodiment, a concentration of cells from about 75, 80, 85, 90, 95, or 100 million cells/ml is used. In further embodiments, concentrations of about 125 or about 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing about 20% DMSO and about 8% human serum albumin, or other suitable cell freezing media. The cells are then frozen to about −80° C. at a rate of about 1 per minute and stored in the vapor phase of a liquid nitrogen storage tank. Other methods of controlled freezing may be used as well as uncontrolled freezing immediately at about −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

Expansion of T Cells

As demonstrated by the data disclosed herein, expanding the T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is about 70% or greater before passing the cells to another culture apparatus. More preferably, the level of confluence is about 90% or greater. A period of time can be any time suitable for the culture of cells in vitro. The T cell medium may be replaced during the culture of the T cells at any time. Preferably, the T cell medium is replaced about every about 2 to 3 days. The T cells are then harvested from the culture apparatus whereupon the T cells can be used immediately or cryopreserved to be stored for use at a later time. In one embodiment, cryopreserving the expanded T cells is provided. The cryopreserved T cells are thawed prior to introducing nucleic acids into the T cell.

In another embodiment, the method comprises isolating T cells and expanding the T cells. In another embodiment, the method further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion can be an alternative or in addition to other methods of expansion described herein. Briefly, ex vivo culture and expansion of T cells comprises the addition to the cellular growth factors or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as about 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example about 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore, the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., about 37° C.) and atmosphere (e.g., air plus about 5% CO₂).

The medium used to culture the T cells may include an agent that can co-stimulate the T cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more by culturing the electroporated population.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

Methods of Treatment

The present invention includes methods for treating cancer, autoantibody or alloantibody diseases or conditions in a subject. In one embodiment, after tumor suppression or B cell depletion by the modified T cells, or following the onset of an adverse reaction to the engineered T cells, the engineered T cells are then selectively ablated in the subject by activating via a dimerization agent, a suicide gene that has been inserted into the engineered T cells.

In one aspect, the invention includes a method for assessing the efficacy or toxicity of an adoptive cell therapy. The modified engineered T cell comprises a nucleic acid encoding a chimeric antigen receptor, comprises a suicide gene and a radiolabeled tracer.

In one embodiment, the method further comprises inducing expression of the suicide gene to produce a suicide gene product that induces cell death of the modified T cell. In one such embodiment, administering an inducing agent induces expression of the suicide gene. In another embodiment, inducing expression of the suicide gene occurs after the modified T cell exerts cytotoxic function against B cells or after an onset of an adverse reaction in the subject to the modified T cell.

In one embodiment, the ligand binding domain is selected from the group consisting of FKBP, F36V-FKBP, and eDHFR.

In another embodiment, the ligand is a monomer or a dimer radiolabeled tracer. In some embodiments, the radiolabeled tracer is a monomer that does not activate the suicide gene.

In one embodiment, the radiolabeled tracer is selected from the group consisting of [¹¹C]-Shield-1; [¹⁸F]-Shield-1; [¹¹C]-Trimethoprim ([¹¹C]-TMP) and [¹⁸F]-Trimethoprim ([¹⁸F]-TMP).

In another embodiment, the method further comprises activating a suicide gene product of the suicide gene to induce cell death of the modified T cell. In one such embodiment, administering an activating agent to promote dimerization of the suicide gene product to activate the suicide gene product. In another embodiment, activating the suicide gene product occurs after the modified T cell exerts cytotoxic function against B cells or after an onset of an adverse reaction in the subject to the modified T cell or after therapeutic effect is achieved.

In yet another embodiment, the method further comprises inhibiting expression of the suicide gene to inhibit cell death of the modified T cell. In one such embodiment, administering an inhibiting agent inhibits expression of the suicide gene. In another embodiment, administering the inhibiting agent occurs concurrently with administration of the modified T cell and continues as the modified T cell exerts cytotoxic function against B cells and may be ceased after an onset of an adverse reaction in the subject to the modified T cell.

The modified T cells can be administered to an animal, preferably a mammal, even more preferably a human, to suppress a tumor or an immune reaction, such as those common to an autoimmune disease or condition or an alloantibody disease or condition.

In addition, the modified T cells of the present invention can be used for the treatment of any condition in which a diminished or otherwise inhibited immune response, especially a cell-mediated immune response, is desired in order to treat or alleviate the disease.

Pharmaceutical Compositions

Pharmaceutical compositions may comprise the modified T cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, compositions are formulated for intravenous administration.

Pharmaceutical compositions may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one aspect, pharmaceutical compositions formulated for use in the methods are described herein, the composition comprising an engineered cell comprising a nucleic acid encoding a suicide gene and a nucleic acid encoding a chimeric antigen receptor comprising an anti-B cell binding domain, a transmembrane domain, a costimulatory domain and an intracellular signaling domain and a ligand comprising a radiolabeled tracer. In some embodiments, the radiolabeled tracer is administered at very low concentrations, so its occupies 1-5% of the available target receptors. In some embodiments, the administered radiolabeled tracer does not have a measurable physiological effect on the subject.

The cells may be administered may be autologous, allogeneic or xenogeneic with respect to the subject undergoing therapy.

Cells can be administered in dosages and routes and at times to be determined in appropriate pre-clinical and clinical experimentation and trials. Cell compositions may be administered multiple times at dosages within these ranges. Administration of the cells may be combined with other methods useful to treat the desired disease or condition as determined by those of skill in the art.

The methods described herein may include administering the composition described herein via a combination therapy in prior to, concurrently with, or subsequent to another medication such as a chemotherapeutic. Accordingly, encompassed is a method of administration of chemotherapeutics, radiation, and/or immunotherapy in conjunction with the composition described herein. In one embodiment, the composition and chemotherapeutic, radiation, and/or immunotherapy are administered to the patient by one or more selected routes of administration sequentially. In another embodiment, a chemotherapeutic agent, radiation, and/or immunotherapy is administered before treatment with a composition described herein. In another embodiment, a chemotherapeutic agent, radiation, and/or immunotherapy is administered after treatment with the composition described herein. In still another embodiment, a chemotherapeutic agent, radiation, and/or immunotherapy is administered during treatment with a composition described herein.

It can generally be stated that a pharmaceutical composition comprising the modified T cells described herein may be administered at a dosage of about 10⁴ to 10⁹ cells/kg body weight, in some instances about 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the modified T cells may be carried out in any convenient manner known to those of skill in the art. The cells may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells are injected directly into a site of inflammation in the subject, a local disease site in the subject, a lymph node, an organ, a tumor, and the like.

In certain embodiments, the modified T cells are administered to a subject. Subsequent to administration, blood is drawn or apheresis is performed, and T cells are modified and expanded therefrom using the methods described here, and are then infused back into the patient. This process can be carried out multiple times every few weeks. In certain embodiments, T cells can be modified from blood draws of from about 10 cc to about 400 cc. In certain embodiments, T cells are modified from blood draws of about 20 cc, 30 cc, 40 cc, 50 cc, 60 cc, 70 cc, 80 cc, 90 cc, or 100 cc. Not to be bound by theory, using this multiple blood draw/multiple reinfusion protocol, may select out certain populations of T cells.

It should be understood that the method and compositions are not limited to the particular formulations set forth in the examples. The following examples are put forth so as to provide those of ordinary skill in the art with a complete disclosure and description of how to make and use the cells, expansion and culture methods, and therapeutic methods, and are not intended to limit the scope described herein.

Unless otherwise indicated, conventional techniques of molecular biology (including recombinant techniques), microbiology, cell biology, biochemistry and immunology, are utilized which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides, and, as such, may be considered. Particularly useful techniques for particular embodiments will be discussed in the sections that follow.

The Compounds

The TMP radiolabeled compounds described have high specificity for DHFR at very low concentrations. The compounds discussed herein have a wide range of uses. In one embodiment, the compounds are capable of imaging natural or engineered immune cells. In another embodiment, the compounds may be used as a positron emission tomography (PET) reporter probe for imaging engineered cells. The binding and retention of TMP by Ec DHFR will allow for clearance of non-bound probe providing high contrast imaging of cells carrying Ec DIFR in a mammal such as but not limited to a human. The ability to longitudinally and non-invasively monitor transgene expression in engineered cells used for cancer immunotherapy would be a powerful, groundbreaking advance beyond current standard PET imaging technologies.

The compounds herein are radiolabeled with a radiotracer ligand. The radiotracer ligand is one that is sufficiently stable to permit binding to TMP and subsequent administration to a patient. In one embodiment, the compound is a radiolabeled TMP. In another embodiment, the compound has the structure of formula (I) or a pharmaceutically acceptable salt or prodrug thereof:

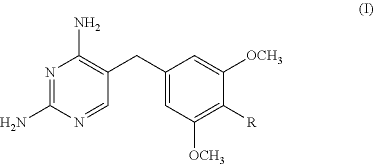

In this structure, R is —O—C¹¹—(C₁ to C₆ alkyl), an O—C¹¹-glycol, O—(C₁ to C₆ alkyl)-¹⁸F, —OCH₂-(¹⁸F substituted phenyl), —OCH₂CH₂-(¹⁸F-substituted triazole), —OCH₂CH₂-(¹⁸F-substituted tetrazole), ¹⁸F-substituted boron-dipyrromethene, —O(CH₂CH₂O)ₙCH₂CH₂-¹⁸F, —OCH₂CH₂CH₂NHC(O)(CH₂CH₂O)ₙ—CH₂CH₂-¹⁸F, -L¹-⁶⁸Ga, -L¹-⁶⁴Cu, -L¹-^{99m}Tc, radioactive halogen, ²¹¹At, -L¹-¹⁰B, -L¹-³²P, -L-⁹⁰Y-L-¹⁰³Pd, -L¹-¹³¹Cs, -L¹-¹⁵³Sm, -L¹-¹⁷⁷Lu, -L¹-²¹¹At, -L¹-²¹²Bi, -L¹-²¹²Po, -L¹-²¹²Pb, -L¹-²²³Ra, or -L¹-²²⁵Ac; n is 1-3; and L is a linker; or a pharmaceutically acceptable salt or prodrug thereof.

In one embodiment, the radioactive halogen is ¹⁸F, ¹²³I, ¹²⁵I, ¹²⁴I, ¹³¹I, ³²Cl, ³³Cl, ³⁴Cl, ⁷⁴Br, ⁷⁵Br, ⁷⁶Br, ⁷⁷Br, or ⁷⁸Br.

In another embodiment, R is O¹¹CH₃.

In a further embodiment, R is ¹⁸F.

In yet other embodiments, L¹ is DTPA, HEHA, NOTA, DOTA, CHX-A, or TCMC.

In yet another embodiment, the compound has the following structure:

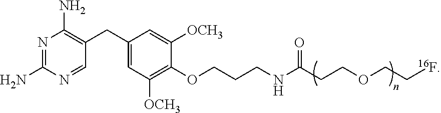

In still a further embodiment, the compound has the following structure:

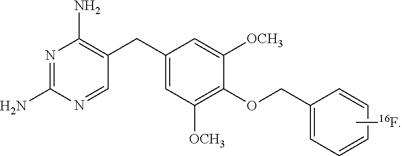

In another embodiment, the compound has the following structure:

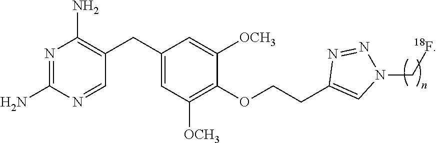

In a further embodiment, the compound has the following structure:

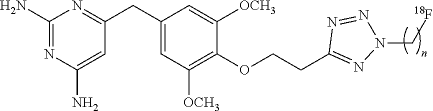

In another embodiment, the compound has the following structure:

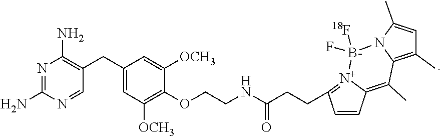 [SMILES: COc1cc(Cc2cnc(N)nc2N)cc(OC)c1OCCNC(=O)CCC1=[N+]2C(=C(C)c3c(C)cc(C)n3[B-]2(F)[18F])C=C1]

In yet a further embodiment, the compound has the following structure:

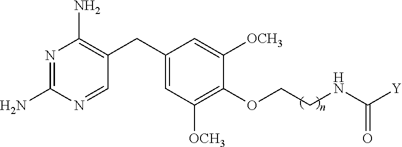

wherein, n is 1 to 6; and Y is

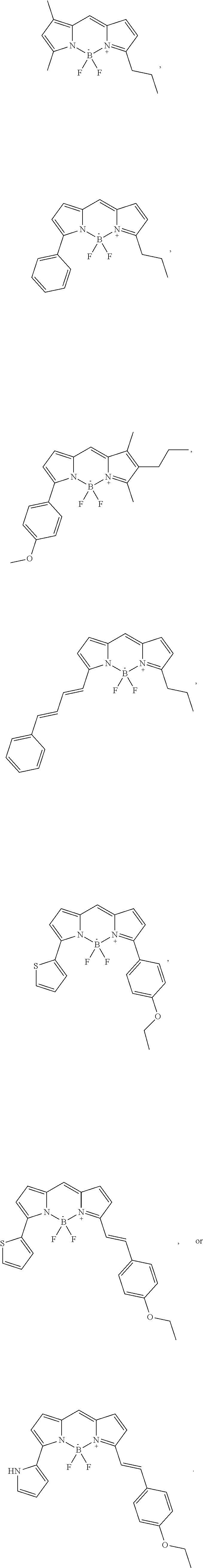

In still another embodiment, ⁶⁴Cu, ⁶⁸Ga, ¹⁰B, ³²P, ⁹⁰Y, ¹⁰³Pd, ¹³¹Cs, ¹⁵³Sm, ¹⁷⁷Lu, ²¹¹At, ²¹²Bi, ²¹²Po, ²¹²Pb, ²²³Ra, or ²²⁵Ac is chelated.

In a further embodiment, wherein the chelation is performed using:

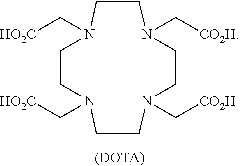

In another embodiment, the chelation is performed using:

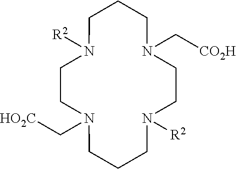

wherein, R² is, independently, H or CH₂CO₂H.

In a further embodiment, the chelation is performed using:

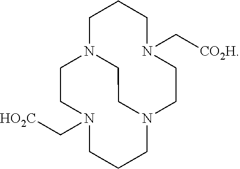 [SMILES: O=C(O)CN1CCCN2CCN(CCCN(CC(=O)O)CC2)CC1]

In another embodiment, the chelation is performed using:

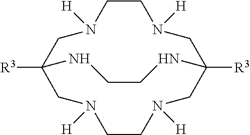

wherein, R³ is, independently, H or NH₂.

In yet a further embodiment, the chelation is performed using:

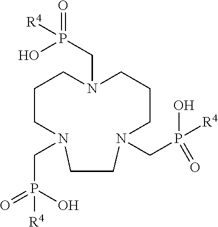

wherein, R⁴ is, independently, H, —(CH₂)₂CO₂H, CH₂OH, or phenyl.

In still another embodiment, the chelation is performed using:

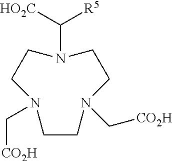

wherein, R⁵ is H or —(CH₂)₂CO₂H.

In a further embodiment, the chelation is performed using:

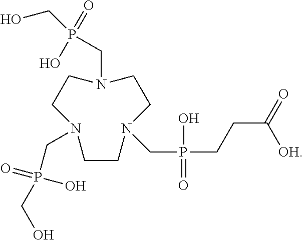 [SMILES: O=C(O)CCP(=O)(O)CN1CCN(CP(=O)(O)CO)CCN(CP(=O)(O)CO)CC1]

In yet a further embodiment, the chelation is performed using:

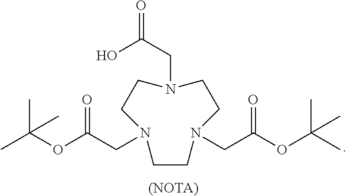

In still another embodiment, the chelation is performed using:

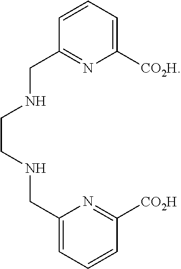 [SMILES: O=C(O)c1cccc(CNCCNCc2cccc(C(=O)O)n2)n1]

In a further embodiment, R is:

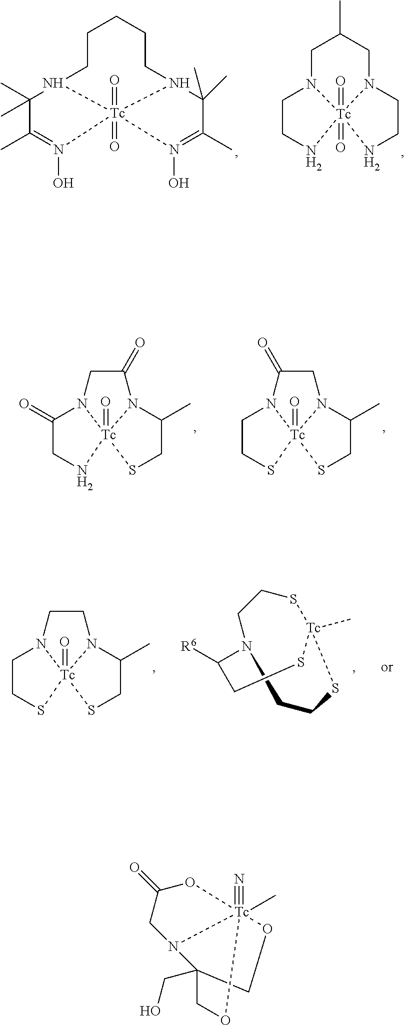

wherein, R⁶ is alkyl or alkoxy.

In this structure, R contains a radioactive isotope. In one embodiment, the radioactive isotope is bound directly to the TMP base molecule. In another embodiment, the radioactive isotope is bound to the base molecule through another chemical moiety. In a further embodiment, the radioactive isotope is a radiolabeled halogen, radiolabeled alkoxy, radiolabeled, glycol, or radiolabeled alkyl group. In yet another embodiment, the radiolabel is —O¹¹—(C₁ to C₆ alkyl), a -glycol-¹¹CH₃, —(C₁ to C₆ alkyl)-¹⁸F, ¹⁸F, ¹²³I, ¹²⁵I, ¹²⁴I, ¹³¹I, ³²Cl, ³³Cl, ³⁴Cl, ⁷⁴Br, ⁷⁵Br, ⁷⁶Br, ⁷⁷Br, ⁷⁸Br, ⁶⁴Cu and ⁶⁸Ga, or radiotherapeutic radioisotopes including ¹⁰B, ³²P, ⁹⁰Y, ¹⁰³Pd, ¹³¹Cs, ¹⁵³Sm, ¹⁷⁷Lu, ²¹¹At, ²¹²Bi, ²¹²Po, ²¹²Pb, ²²³Ra, or ²²⁵Ac. In still a further embodiment, the radiolabel is O-¹¹CH₃ or CH₂CH₂CH₂¹⁸F. In yet another embodiment, the radiolabel is —(C₁ to C₆ alkoxy amido)-chelated ⁶⁴Cu or —(C₁ to C₆ alkoxy amido)-chelated ⁶⁸Ga. Instill a further embodiment, the radiolabel is -DOTA-⁶⁴Cu or -NOTA-⁶⁸Ga. In other embodiments, the radiotherapeutic radioisotope is chelated.

The term “glycol” as used herein refers to an organic compound have two hydroxyl groups, each hydroxyl being attached to different carbon atoms. In one embodiment, the glycol is ethylene glycol, propylene glycol, 1,3-butanediol, 1,4-butanediol, 2-ethyl-1,3-hexanediol, 2-methyl-2-propyl-1,3-propanediol, polyglycols, among others. One or more hydrogen bound to the carbon atom may be substituted with a radioactive isotope described herein.

The term “alkyl” is used herein to refer to both straight- and branched-chain saturated aliphatic hydrocarbon groups. In one embodiment, an alkyl group has 1 to about 10 carbon atoms. In another embodiment, an alkyl group has 1 to about 6 carbon atoms. In a further embodiment, an alkyl group has 1 to about 4 carbon atoms. The alkyl group may be optionally substituted with one or more radiolabel as provided above.

The term “alkoxy” as used herein refers to the O-(alkyl) group, where the point of attachment is through the oxygen-atom and the alkyl group is defined above. The alkoxy group may be optionally substituted with one or more radiolabel as provided above.

The term “halogen” as used herein refers to Cl, Br, F, or I groups.

Methods of Production

The compounds described above may be prepared by known chemical synthesis techniques. Among such preferred techniques known to one of skill in the art are included the synthetic methods described in conventional textbooks relating to the construction of synthetic compounds. [¹¹C]Trimethoprim ([¹¹C]TMP) and [¹⁸F]Fluoropropyltrimethoprim ([¹⁸F]FP-TMP) may be prepared as described in the examples and according to the following.

A. [¹¹C]TMP

[¹¹C]TMP was prepared using skill in the art and the steps outlined in Scheme 1 and Example 1.

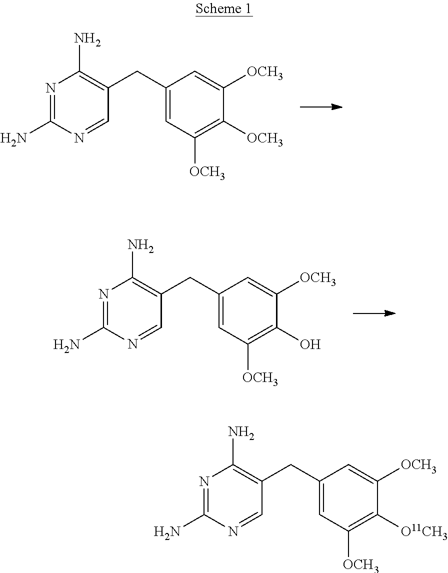

[¹¹C]TMP was prepared using intermediate 5-(3,5-dimethoxy-4-hydroxy-benzyl)pyrimidine-2,4-diamine using the procedure in Chem. Bio. Chem., 2007, 8:767-774. See, Scheme 1. In one embodiment, trimethoprim (3 g, 10.03 mmol, purchased from Sigma-Aldrich) is selectively demethylated by HBr (48% in water) for 20 min at 95° C. After the reaction, the reaction mixture was cooled down and NaOH (8.92 mL, 50% w/w) added. After precipitation, the precipitate was filtered and collected, and re-dissolved in boiling water. NH₄OH was added to the mixture solution until pH 7, recrystallized at 4° C., filtered, and collected. 5-(3,5-dimethoxy-4-hydroxy-benzyl)pyrimidine-2,4-diamine was obtained as a pink solid (1.51 g) in 52.9% yield; ¹H NMR (DMSO-d₆) δ 8.06 (s, —OH), 7.45 (s, 1H), 6.48 (s, 2H), 5.99 (s, —NH₂), 5.63 (s, —NH₂), 3.71 (s, 6H), 3.47 (s, 2H). Followed by next step, 5-(3,5-dimethoxy-4-hydroxy-benzyl)pyrimidine-2,4-diamine was reacted with [¹¹C]CH₃I for 5 min at 70° C. in the present of 5 N NaOH (4 μL) as a base. After the reaction, the reaction mixture was purified by HPLC with 12% EtOH in 0.01 M Phosphate buffer (pH=3). The flow rate of HPLC was 3 mL/min and the product ([¹¹C]TMP) was eluted at 12 min retention time. The radiochemical yield was 40-50% from [¹¹C]CH₃I, radiochemical purity was over 99%, and the specific activity was 37-56 GBq/μmol.

[¹¹C]TMP has a half-life of about 20.4 minutes. In one embodiment, [¹¹C]TMP is synthesized at a facility having a cyclotron.

There are also key advantages to using [¹¹C]TMP as a radiotracer. First, the synthetic route to [¹¹C]TMP is only two-steps and facile. The precursor is inexpensive and widely available. TMP as an antibiotic is often used clinically in combination with sulfamethoxazole and thus the toxicity profile is well known. Since [¹¹C]TMP is the same chemical structure as the unlabeled antibiotic, this tracer may be rapidly applied into patients. TMP radiotracer imaging may have a much lower background as the target (bacterial DHFR) is not native to human cells. This leads to high sensitivity imaging, for example 3×10⁵ cells implanted were detected, which is a clinically relevant concentration based on current therapies which typically implant greater than 10⁷ cells.

Other advantages of [¹¹C]TMP include the ability to possibly cross the blood brain barrier. By doing so, [¹¹C]TMP may be utilized for visualizing cells in areas of high background for reporter proteins derived from nervous system tissue (e.g. hNET and D2R) or in cases where alternative small molecule probes do not adequately cross the blood brain barrier.

Other radiolabeled alkyl groups may be incorporated into the para position in place of ¹¹CH₃. In one embodiment, one or more carbon atoms of the alkyl group contains a ¹C atom.

B. [¹⁸F]FP-TMP

The inventors also found the synthesis route of ¹⁸F-radiolabeled derivatives of TMP. Of importance, the inventors finally found that substituting the O-atom at the para position of the benzene ring with a longer chain permitted ¹⁸F labeling. In one embodiment, the para-position O-atom is substituted with an alkoxylated silate. In another embodiment, the para-position O-atom was substituted with 3-bromopropoxy-tert-butyldimethyl silate. This step is important to attach alkoxy linker of 4-hydroxyl group in benzene ring for TMP compound, can be purified by flash column and selectively deprotected for TBDMS group without any effect of Boc protecting group. Accordingly, one method of preparing the following ¹⁸F-radiolabeled derivative was performed as described herein.

The inventors found that [¹⁸F]Fluoropropyl trimethoprim has a 110 min half-life. This longer shelf life advantageously permits increased incubation time of the compound with the target bacteria with less loss of signal due to isotope decay. Further, this also permits metabolic clearance of the compound.

Compositions Containing the Compound

Pharmaceutical compositions useful herein, in one embodiment, contain a compound discussed above in a pharmaceutically acceptable carrier or diluent with other optional suitable pharmaceutically inert or inactive ingredients. In another embodiment, a compound described above is present in a single composition. In a further embodiment, a compound described above is combined with one or more excipients and/or other therapeutic agents as described below.

(i) Salts

The compounds discussed above may encompass tautomeric forms of the structures provided herein characterized by the bioactivity of the drawn structures. Further, the compounds may also be used in the form of salts derived from pharmaceutically or physiologically acceptable acids, bases, alkali metals and alkaline earth metals.

In one embodiment, pharmaceutically acceptable salts can be formed from organic and inorganic acids including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids.

In another embodiment, pharmaceutically acceptable salts may also be formed from inorganic bases, desirably alkali metal salts including, e.g., sodium, lithium, or potassium, such as alkali metal hydroxides. Examples of inorganic bases include, without limitation, sodium hydroxide, potassium hydroxide, calcium hydroxide, and magnesium hydroxide. Pharmaceutically acceptable salts may also be formed from organic bases, such as ammonium salts, mono-, di-, and trimethylammonium, mono-, di- and triethylammonium, mono-, di- and tripropylammonium, ethyldimethylammonium, benzyldimethylammonium, cyclohexylammonium, benzyl-ammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1-n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmono-ethanolammonium, diethanolamine, ethylenediamine, and the like. In one example, the base is selected from among sodium hydroxide, lithium hydroxide, potassium hydroxide, and mixtures thereof.

(ii) Prodrugs

The salts, as well as other compounds, can be in the form of esters, carbamates and other conventional “pro-drug” forms, which, when administered in such form, convert to the active moiety in vivo. In one embodiment, the prodrugs are esters. In another embodiment, the prodrugs are carbamates. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996), which is incorporated by reference.

(iii) Carriers and Diluents

The pharmaceutical compositions include a compound described herein formulated neat or with one or more pharmaceutical carriers for administration, the proportion of which is determined by the solubility and chemical nature of the compound, chosen route of administration and standard pharmacological practice. The pharmaceutical carrier may be solid or liquid.

The compound may be administered by any desirable route, taking into consideration the specific condition for which it has been selected. The compound may, therefore, be delivered orally, by injection, i.e., transdermal, intravenous, subcutaneous, intramuscular, intravenous, intra-arterial, intraperitoneal, intrathecal, intracavitary, or epidural, among others.

Although the compound may be administered alone, it may also be administered in the presence of one or more pharmaceutical carriers that are physiologically compatible. The carriers may be in dry or liquid form and must be pharmaceutically acceptable. Liquid pharmaceutical compositions are typically sterile solutions or suspensions.

When liquid carriers are utilized, they are desirably sterile liquids. Liquid carriers are typically utilized in preparing solutions, suspensions, emulsions, syrups and elixirs. In one embodiment, the compound is dissolved a liquid carrier. In another embodiment, the compound is suspended in a liquid carrier. One of skill in the art of formulations would be able to select a suitable liquid carrier, depending on the route of administration. In one embodiment, the liquid carrier includes, without limitation, water, organic solvents, oils, fats, or mixtures thereof. In another embodiment, the liquid carrier is water containing cellulose derivatives such as sodium carboxymethyl cellulose. In a further embodiment, the liquid carrier is water and/or dimethylsulfoxide. Examples of organic solvents include, without limitation, alcohols such as monohydric alcohols and polyhydric alcohols, e.g., glycols and their derivatives, among others. Examples of oils include, without limitation, fractionated coconut oil, arachis oil, corn oil, peanut oil, and sesame oil and oily esters such as ethyl oleate and isopropyl myristate.

Alternatively, the compound may be formulated in a solid carrier. In one embodiment, the composition may be compacted into a unit dose form, i.e., tablet or caplet. In another embodiment, the composition may be added to unit dose form, i.e., a capsule. In a further embodiment, the composition may be formulated for administration as a powder. The solid carrier may perform a variety of functions, i.e., may perform the functions of two or more of the excipients described below. For example, the solid carrier may also act as a flavoring agent, lubricant, solubilizer, suspending agent, filler, glidant, compression aid, binder, disintegrant, or encapsulating material. Suitable solid carriers include, without limitation, calcium phosphate, dicalcium phosphate, magnesium stearate, talc, starch, sugars (including, e.g., lactose and sucrose), cellulose (including, e.g., microcrystalline cellulose, methyl cellulose, sodium carboxymethyl cellulose), polyvinylpyrrolidine, low melting waxes, ion exchange resins, and kaolin. The solid carrier can contain other suitable excipients, including those described below.

Examples of excipients which may be combined with the compound include, without limitation, adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjustors, preservatives, solubilizers, sorbents, stabilizers, sweeteners, surfactants, suspending agents, syrups, thickening agents, or viscosity regulators. See, the excipients described in the “Handbook of Pharmaceutical Excipients”, 5^{th} Edition, Eds.: Rowe, Sheskey, and Owen, APhA Publications (Washington, D.C.), Dec. 14, 2005, which is incorporated herein by reference.

Methods of Using the Compound

As discussed above, the radiolabeled compound have potential in a wide scope of applications including, without limitation, imaging inflammation, infection, immunotherapy, and treating cancer. As described herein, a therapeutically or prophylactically effective amount of a compound is that amount of a compound which provides a sufficient amount of radiation. The effective amount of may be determined by the attending physician, formulation and route of delivery, condition treated, compound, route of delivery, age, weight, severity of the patient's symptoms, and response pattern of the patient. In one embodiment, effective amount does not exceed normal organ dose limits. In one embodiment, the effective amount is about 0.01 mg/kg to 10 mg/kg body weight. In another embodiment, the effective amount is less than about 5 g/kg, about 500 μg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 100 μg/kg, about 75 μg/kg, about 50 μg/kg, about 25 μg/kg, about 10 μg/kg, or about 1 μg/kg.

A therapeutically or prophylactically effective amount of a compound may also be that amount of a compound which provides a sufficient amount of radiation. The sufficient amount of radiation may vary depending upon the formulation and route of delivery. In one embodiment, the amount (i.e., per unit) of the compound is that which does not exceed normal organ dose limits. In one embodiment, the compound delivers about 1 μCi to about 100 mCi of radiation. In other embodiments, the compound delivers about 1 μCi to about 50 mCi, or about 1 μCi to about 10 mCi, of radiation. However, the effective amount to be used is subjectively determined by the attending physician and variables such as the size, age and response pattern of the patient.

These effective amounts may be provided on regular schedule, i.e., daily, weekly, monthly, or yearly basis or on an irregular schedule with varying administration days, weeks, months, etc. Alternatively, the effective amount to be administered may vary. In one embodiment, the effective amount for the first dose is higher than the effective amount for one or more of the subsequent doses. In another embodiment, the effective amount for the first dose is lower than the effective amount for one or more of the subsequent doses.

Imaging Methods

The compounds discussed herein show robust uptake in vitro and in vivo and, and favorable distribution and sensitivity. In some embodiments, the compounds permit non-invasively monitoring of less than 0.5 million engineered cells, which is an advance beyond the current PET report gene technologies.

In some embodiments, the compounds and methods described herein permit distinguishing diseases/conditions which are often misdiagnosed as being due to bacterial infection. In one embodiment, the compounds and methods distinguish chemical or aspiration pneumonitis from bacterial pneumonia.

A number of different genera and species of bacteria may be imaged using the compounds and methods discussed herein. In one embodiment, the bacteria are sensitive to trimethoprim. In another embodiment, the bacterium is E. coli, S. aureus, P. aureginosa, Enterobacter, Haemophilus, Klebsiella, Morganella, Proteus, Providencia, Salmonella, Serratia, Streptococcus A, Streptococcus B, Streptococcus C, Streptococcus G, Mycobacterium TB, or any combination thereof. The bacteria may be commensal or infections. In one embodiment, the bacterial are gastrointestinal tract bacteria.

Not only are the compounds and methods useful in tracking bacteria, but they may be used in methods of monitoring treatment of a bacterial infection in a subject. In doing so, an antibiotic is administered to the subject using antibiotics known in the art. In one embodiment, the antibiotic is trimethoprim. An effective amount of a compound described may then be administered to the subject and the compound tracked to determine an approximate location of the infection. Alternatively, the compound is administered to the subject prior to administration of the antibiotic.

Accordingly, methods of imaging immune cells, such as but not limited to T cells and CAR T cells in a subject are provided. The method includes administering an effective amount of a compound described herein and (b) tracking the radiolabeled derivative. By doing so, methods of tracking or monitoring the cells are provided.

Immunotherapy Methods

The compounds discussed herein may be used for imaging therapeutic cells and in methods of immunotherapy. The methods include genetically engineering cells from the patient to express dihydrofolate reductase. The engineered cells may then be tagged with a compound described herein. In one embodiment, the cells are transgenic cells carrying E. coli DHFR. The genetically engineered and tagged cells may then be administered to the patient and tracked using techniques such as imaging.

Such genetic engineering may be performed using skill in the art. In one embodiment, the genetically engineering cells are T-cells, NK-cells, macrophages, B-cells, stem cells, hematopoietic stem cells, mesenchymal stem cells, neuroprogenitor cells, induced pluripotent cells, or any combinations thereof. In one embodiment, the genetically engineering cells are CAR T cells.

In one embodiment, the compounds may be used for tracking or treating cancer or autoimmune diseases, immune deficiency related diseases (e.g. HIV) or other immune related conditions.

Additional Uses

The compounds discussed herein also have use in other applications. Accordingly, the compounds may be used in “problem-solving”, i.e., when physician have difficulties determining a medical condition in a patient and traditional laboratory and imaging diagnostics have failed. For example, such conditions include, without limitation, cases of osteomyelitis or fever of unknown origin.

A significant “problem” which may be addressed by the compounds discussed herein include cancer. Accordingly, methods of treating cancer in a subject are provided and include initiating treatment of the cancer. In one embodiment, the subject is treated using a chemotherapeutic, radiation, or immunotherapy. The subject is then administered an effective amount of a compound described herein and the compound is tracked using imaging as previously described.

As noted above, the methods described herein may include administering a compound described herein via a combination therapy in prior to, concurrently with, or subsequent to another medication such as a chemotherapeutic. Accordingly, encompassed is a method of administration of chemotherapeutics, radiation, and/or immunotherapy in conjunction with a compound described herein. In one embodiment, the compound and chemotherapeutic, radiation, and/or immunotherapy are administered to the patient by one or more selected routes of administration sequentially. In another embodiment, a chemotherapeutic agent, radiation, and/or immunotherapy is administered before treatment with a compound described herein. In another embodiment, a chemotherapeutic agent, radiation, and/or immunotherapy is administered after treatment with a compound described herein. In still another embodiment, a chemotherapeutic agent, radiation, and/or immunotherapy is administered during treatment with a compound described herein.

The chemotherapeutic, radiation, and/or immunotherapy used to treat the cancer may be selected by one skill in the art.

In another embodiment, the compounds may be utilized in when the bacterial DHFR DNA is genetically attached to any gene of interest. Specifically, after standard transcription and translation, the protein target of the compound, bacterial DHFR, may be attached to the protein of interest. Imaging the compound provides the cellular concentration of the protein of interest. Advantageously, the genetic fusion of DHFR to a protein of interest does not affect protein of interest function. Examples of proteins include, without limitation, RAS, RAC, BAX, BRCA1, BRCA2, P53, RB, RHO, MTOR, or CAS9. See, Iwamoto, Chem. Biol., 17:981-988, 2010.

In a further embodiment, the compounds may be used as probes. In one embodiment, the probe is a PET reporter probe. In another embodiment, the compounds may be used prior to or after surgeries in an effort to determine if there is an existing bacterial infection and/or inflammation. The use of the compounds may be for a number of different types of surgeries including, without limitation, orthopedic surgery, abdominal, biliary, pancreatic, breast, prostate, GI, GU, endocrine, oncologic, neurologic, vascular, and podiatric surgery.

Kits Containing the Compound

Also provided herein are kits or packages of pharmaceutical formulations containing a compound or composition described herein. The kits may be organized to indicate a single formulation or combination of formulations to be taken at each desired time. The composition may also be sub-divided to contain appropriate quantities of the compound. For example, the unit dosage can be packaged compositions, e.g., packeted powders, vials, ampoules, prefilled syringes or sachets containing liquids.

Suitably, the kit contains packaging or a container with the compound formulated for the desired delivery route. Suitably, the kit contains instructions on dosing and an insert regarding the compound. Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. For example, the kit may also contain instructions for use of the delivery device. Other suitable components to include in such kits will be readily apparent to one of skill in the art, taking into consideration the desired indication and the delivery route. The doses are repeated daily, weekly, or monthly, for a predetermined length of time or as prescribed.

The compound or composition described herein can be a single dose or for continuous or periodic discontinuous administration. For continuous administration, a package or kit can include the compound in each dosage unit, e.g., solution, lotion, tablet, pill, or other unit described above or utilized in drug delivery. When the compound is to be delivered with periodic discontinuation, a package or kit can include placebos during periods when the compound is not delivered. When varying concentrations of a composition, of the components of the composition, or of relative ratios of the compound or other agents within a composition over time is desired, a package or kit may contain a sequence of dosage units, so varying.

A number of packages or kits are known in the art for the use in dispensing pharmaceutical agents for oral use. In one embodiment, the package has indicators for each period. In another embodiment, the package is a labeled blister package, dial dispenser package, or bottle.

The packaging means of a kit may itself be geared for administration, such as an inhalant, syringe, pipette, eye dropper, or other such like apparatus, from which the formulation may be applied to an infected area of the body, such as the lungs, injected into a subject, or even applied to and mixed with the other components of the kit.

The compound or composition of these kits also may be provided in dried or lyophilized forms. When reagents or components are provided as a dried form, reconstitution generally is by the addition of a suitable solvent. It is envisioned that the solvent also may be provided in another packaging means.

The kits may include a means for containing the vials in close confinement for commercial sale such as, e.g., injection or blow-molded plastic containers into which the desired vials are retained.

Irrespective of the number or type of packages, the kits also may include, or be packaged with a separate instrument for assisting with the injection/administration or placement of the ultimate complex composition within the body of an animal. Such an instrument may be an inhalant, syringe, pipette, forceps, measuring spoon, eye dropper or any such medically approved delivery means. Other instrumentation includes devices that permit the reading or monitoring of reactions in vitro.

In one embodiment, a pharmaceutical kit is provided and contains a compound of formula (I). The compound may be in the presence or absence of one or more of the carriers or excipients described above. The kit may optionally contain a chemotherapeutic and/or instructions for administering the chemotherapeutic and the compound to a subject having cancer.

In a further embodiment, a pharmaceutical kit is provided and contains a chemotherapeutic in a first dosage unit, one or more of a compound selected from those described herein in a second dosage unit, and one or more of the carriers or excipients described above in a third dosage unit. The kit may optionally contain instructions for administering the chemotherapeutic and/or compound to a subject having cancer.

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C., pressure is at or near atmospheric.

### ILLUSTRATIVE EMBODIMENTS

Provided here are illustrative embodiments of the disclosed technology. These embodiments are illustrative only and do not limit the scope of the present disclosure or of the claims attached hereto.

### Embodiments on Radiotracer Derivatives of Trimethoprim for Diagnostic Imaging

### Embodiment 1

A compound having the structure of formula (I), or a pharmaceutically acceptable salt or prodrug thereof, wherein, R is —O¹¹—(C₁ to C₆ alkyl), an O¹¹-glycol, —(C₁ to C₆ alkyl)-¹⁸F, ¹⁸F, ¹²³I, ¹²⁵I, ¹²⁴I, ¹³¹I, ³²Cl, ³³Cl, ³⁴Cl, ⁷⁴Br, ⁷⁵Br, ⁷⁶Br, ⁷⁷Br, ⁷⁸Br, ⁶⁴Cu, ⁶⁸Ga, ¹⁰B, ³²P, ⁹⁰Y, ¹⁰³Pd, ¹³¹Cs, ¹⁵³Sm, ¹⁷⁷Lu, ²¹¹At, ²¹²Bi, ²¹²Po, ²¹²Pb, ²²³Ra, or ²²⁵Ac:

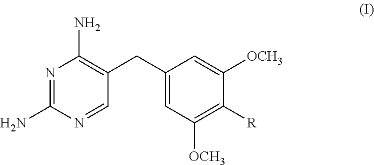

wherein, R is —O—C¹¹—(C₁ to C₆ alkyl), an O—C¹¹-glycol, —O—(C₁ to C₆ alkyl)-¹⁸F, —OCH₂-(¹⁸F substituted phenyl), —OCH₂CH₂-(¹⁸F-substituted triazole), —OCH₂CH₂-(¹⁸F-substituted tetrazole), ¹⁸F-substituted boron-dipyrromethene, —O(CH₂CH₂O)ₙCH₂CH₂-¹⁸F, —OCH₂CH₂CH₂NHC(O)(CH₂CH₂O)ₙ—CH₂CH₂-¹⁸F, -L¹-⁶⁸Ga, -L¹-⁶⁴Cu, -L¹-^{99m}Tc, radioactive halogen, ²¹¹At, -L¹-¹⁰B, -L¹-³²P, -L¹-⁹⁰Y, -L¹-¹⁰³Pd, -L¹-¹³¹Cs, -L¹-¹⁵³Sm, -L¹-¹⁷⁷Lu, - L¹-²¹¹At, -L¹-²¹²Bi, -L¹-²¹²Po, -L¹-²¹²Pb, -L¹-²²³Ra, or -L¹-²²⁵Ac; n is 1-3; L¹ is a linker; or a pharmaceutically acceptable salt or prodrug thereof.

### Embodiment 2

The compound of Embodiment 1, wherein said radioactive halogen is ¹⁸F, ¹²³I, ¹²⁵I, ¹²⁴I, ¹³¹I, ³²Cl, ³³Cl, ³⁴Cl, ⁷⁴Br, ⁷⁵Br, ⁷⁶Br, ⁷⁷Br, or ⁷⁸Br.

### Embodiment 3

The compound of Embodiment 1, wherein R is O¹¹CH₃.

### Embodiment 4

The compound of Embodiment 1, wherein R is ¹⁸F.

### Embodiment 5

The compound of Embodiment 1, which has the following structure:

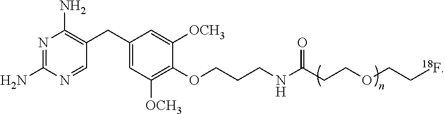

### Embodiment 6

The compound of Embodiment 1 which has the following structure:

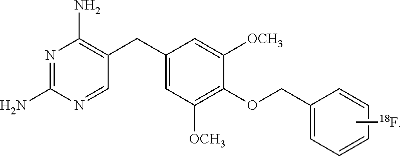

### Embodiment 7

The compound of Embodiment 1, which has the following structure:

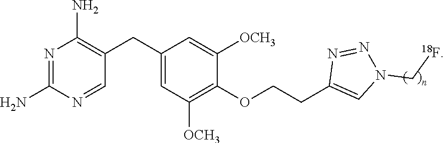

### Embodiment 8

The compound of Embodiment 1, which has the following structure:

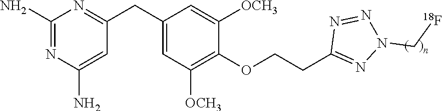

### Embodiment 9

The compound of Embodiment 1 which has the following structure:

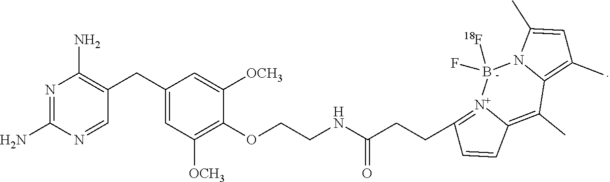

### Embodiment 10

The compound of Embodiment 1, which has the following structure:

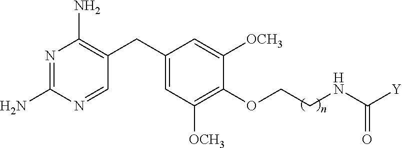

wherein: n is 1 to 6; and

### Y is

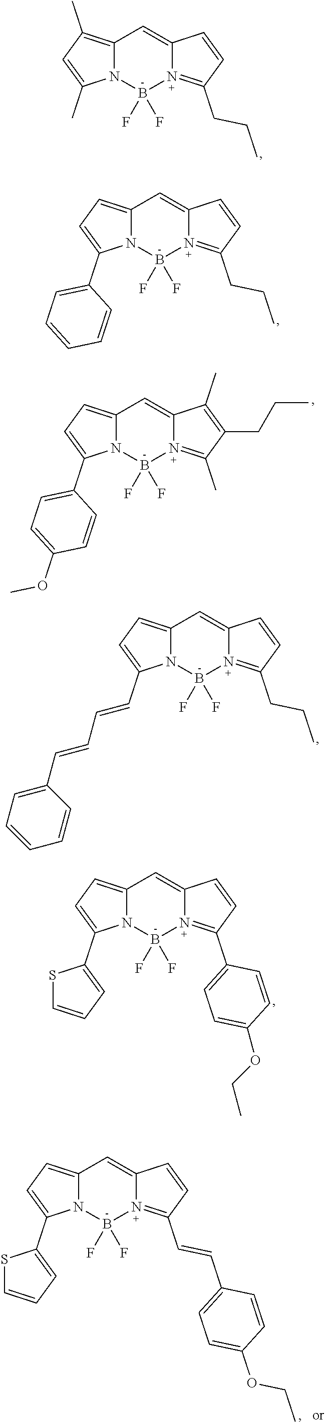

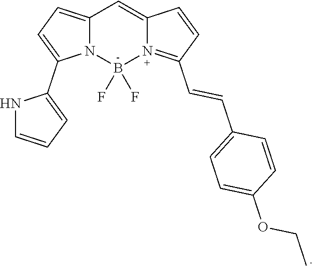

### Embodiment 11

The compound of Embodiment 1, wherein ⁶⁴Cu, ⁶⁸Ga, ¹⁰B, ³²P, ⁹⁰Y, ¹⁰³Pd, ¹³¹Cs, ¹⁵³Sm, ¹⁷⁷Lu, ²¹¹At, ²¹²Bi, ²¹²Po, ²¹²Pb, ²²³Ra, or ²²⁵Ac is chelated.

### Embodiment 12

The compound of Embodiment 11, wherein the chelation is performed using:

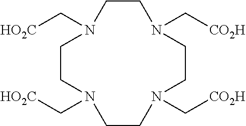 [SMILES: O=C(O)CN1CCN(CC(=O)O)CCN(CC(=O)O)CCN(CC(=O)O)CC1]

### Embodiment 13

The compound of Embodiment 11, wherein the chelation is performed using:

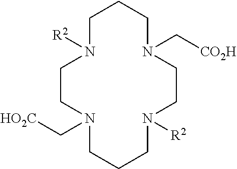

wherein, R² is, independently, H or CH₂CO₂H.

### Embodiment 14

The compound of Embodiment 11, wherein the chelation is performed using:

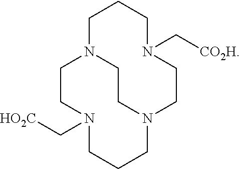 [SMILES: O=C(O)CN1CCCN2CCN(CCCN(CC(=O)O)CC2)CC1]

### Embodiment 15

The compound of Embodiment 11, wherein the chelation is performed using:

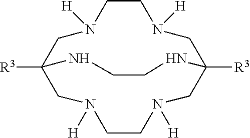

wherein, R³ is, independently, H or NH₂.

### Embodiment 16

The compound of Embodiment 11, wherein the chelation is performed using:

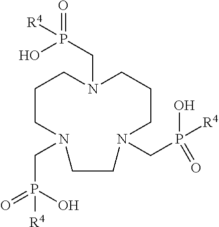

wherein, R⁴ is, independently, H, —(CH₂)₂CO₂H, CH₂OH, or phenyl.

### Embodiment 17

The compound of Embodiment 11, wherein the chelation is performed using:

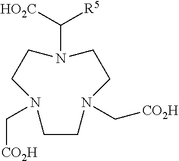

wherein, R⁵ is H or —(CH₂)₂CO₂H.

### Embodiment 18

The compound of Embodiment 11, wherein the chelation is performed using:

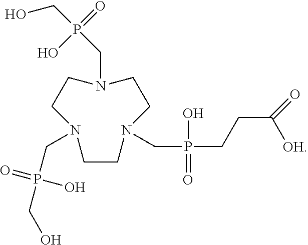 [SMILES: O=C(O)CCP(=O)(O)CN1CCN(CP(=O)(O)CO)CCN(CP(=O)(O)CO)CC1]

### Embodiment 19

The compound of Embodiment 11, wherein the chelation is performed using:

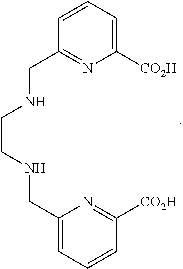 [SMILES: O=C(O)c1cccc(CNCCNCc2cccc(C(=O)O)n2)n1]

### Embodiment 20

The compound of Embodiment 1, wherein R is:

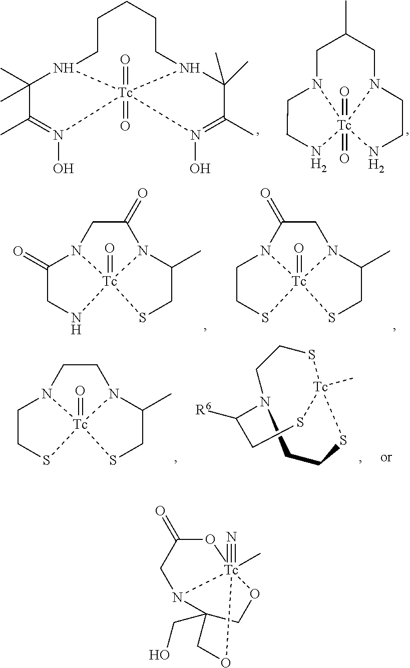

wherein, R⁶ is alkyl or alkoxy.

### Embodiment 21

A composition comprising a compound of any one of Embodiments 1 to 20 and a pharmaceutically acceptable carrier or diluent.

### Embodiment 22

A method of imaging a bacterial infection in a subject, said method comprising (a) administering an effective amount of a compound of any one of Embodiments 1 to 20 to said subject; and (b) tracking said compound.

### Embodiment 23

A method of tracking or monitoring bacteria in a subject, said method comprising (a) administering an effective amount of a compound of any one of Embodiments 1 to 20 to said subject; and (b) tracking said compound.

### Embodiment 24

A method of distinguishing a bacterial infection from inflammation, said method comprising (a) administering an effective amount of a compound of any one of Embodiments 1 to 20 to said subject; and (b) tracking said compound using imaging; wherein said infection is displayed in the image and said inflammation is absent in said image.

### Embodiment 25

The method of Embodiment 24, which distinguishes chemical or aspiration pneumonitis from bacterial pneumonia.

### Embodiment 26

The method of Embodiment 23 or 24, wherein said bacteria are commensal or infectious.

### Embodiment 27

A method of treating a bacterial infection in a subject, said method comprising (a) administering an antibiotic to said subject; (b) administering an effective amount of a compound of any one of Embodiments 1 to 20 to said subject; and (c) tracking said compound.

### Embodiment 28

A method of monitoring cancer treatment in a subject, said method comprising (a) administering a chemotherapeutic, radiation, or immunotherapy to said subject; (b) administering an effective amount of a compound of any one of Embodiments 1 to 20 to said subject; and (c) tracking said compound.

### Embodiment 29

A method of monitoring immunotherapy in a subject, said method comprising (a) genetically engineering cells from said patient to express dihydrofolate reductase; (b) tagging said engineered cells with a compound of any one of Embodiments 1 to 20; (c) administering said genetically engineered and tagged cells to said patient; and (d) tracking said genetically engineering cells and tagged cells by imaging.

### Embodiment 30

The method of Embodiment 29, wherein said cells are T-cells, NK-cells, macrophages, B-cells, stem cells, hematopoietic stem cells, mesenchymal stem cells, neuroprogenitor cells, or induced pluripotent cells.

### Embodiment 31

The method of anyone of Embodiments 22 to 30, wherein said tracking is performed using positron emission tomography or single photon emission computed tomography.

### Embodiment 32

The method of Embodiment 31, wherein the positron emission tomography which does not display areas of inflammation.

### Embodiment 33

The method of any one of Embodiments 22 to 32, wherein said radiolabeled derivative of trimethoprim is administered through oral, intravenous, intra-arterial, intraperitoneal, intrathecal, or intracavitary injection

### Embodiment 34

The method of any one of Embodiments 22 to 27, wherein said bacteria is E. coli, S. aureus, P. aureginosa, Enterobacter, Haemophilus, Klebsiella, Morganella, Proteus, Providencia, Salmonella, Serratia, Streptococcus A, Streptococcus B, Streptococcus C, Streptococcus G, Mycobacterium TB, or any combination thereof.

### Embodiment 35

The method of anyone of Embodiments 22 to 34, wherein said effective amount is about 1 to about 50 mCi.

### Embodiment 36

The method of any one of Embodiments 22 to 27, wherein said bacteria comprises gastrointestinal tract bacteria.

### Embodiment 37

The method of any one of Embodiments 22 to 36, wherein said subject is an animal or human.

### Embodiment 38

A positron emission tomography reporter probe comprising a compound of any one of Embodiments 1 to 20.

### Embodiment 39

A method of preparing the following compound:

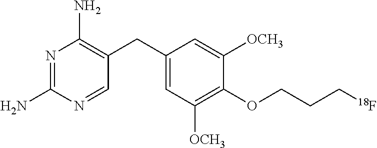 [SMILES: COc1cc(Cc2cnc(N)nc2N)cc(OC)c1OCCC[18F]]

said method comprising (i) reducing trimethoprim to provide

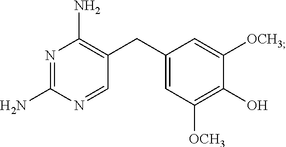 [SMILES: COc1cc(Cc2cnc(N)nc2N)cc(OC)c1O]

(ii) with 3-bromopropoxy-tert-butyldimethyl silate to provide;

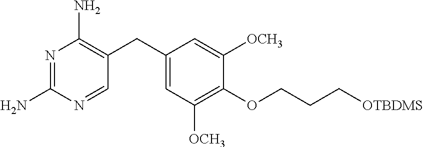 [SMILES: COc1cc(Cc2cnc(N)nc2N)cc(OC)c1OCCCO[Si](C)(C)C(C)(C)C]

iii) protecting with bis-Boc groups each NH₂ group

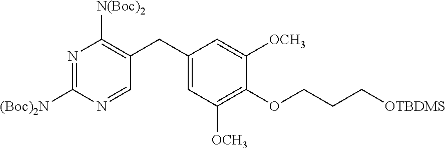

(iii) reacting the product of step (iii) deprotecting the product of step (iv) to provide

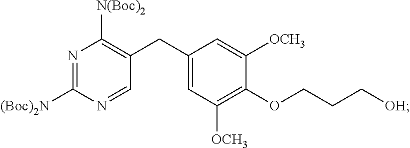

(v) mesylating the OH group of step (vi);

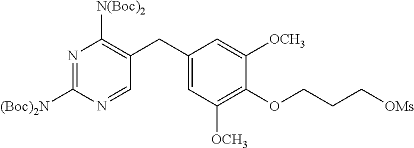

and (vii) replacing the mesylate group with ¹⁸F; and (viii) removing the BOC groups by deprotection.

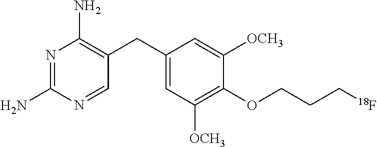 [SMILES: COc1cc(Cc2cnc(N)nc2N)cc(OC)c1OCCC[18F]]

Sfsdf

### Embodiments on Compositions and Methods for Imaging Immunes Cells

### Embodiment 1

An engineered cell comprising a chimeric antigen receptor (CAR) and further comprising a nucleic acid molecule comprising a ligand binding domain capable of binding to a radiolabeled tracer.

### Embodiment 2

The engineered cell of embodiment 1, wherein the cell is a T cell.

### Embodiment 3

The engineered cell of embodiment 1, wherein the ligand binding domain is E. coli dihydrofolate reductase (eDHFR).

### Embodiment 4

The engineered cell of embodiment 1, wherein the radiolabeled tracer is [¹⁸F]fluoropropyl-trimethoprim ([¹⁸F]FPTMP).

### Embodiment 5

A method of assessing the efficacy or toxicity of an adoptive cell therapy in a subject, the method comprising: administering to the subject an engineered T cell comprising a chimeric antigen receptor (CAR) and a nucleic acid molecule comprising a ligand binding domain; administering to the subject a radiolabeled tracer capable of binding to the ligand binding domain; detecting the amount of radiolabeled tracer bound by imaging; and, assessing the efficacy or toxicity of the adoptive cell therapy in the subject.

### Embodiment 6

A method of detecting the quantity of engineered T cells in a subject, the method comprising: administering to the subject an engineered T cell comprising a chimeric antigen receptor (CAR) and a nucleic acid molecule comprising a ligand binding domain; administering to the subject a radiolabeled tracer capable of binding to the ligand binding domain; and, imaging the amount of radiolabeled tracer bound thereby detecting the quantity of engineered T cells in the subject.

### Embodiment 7

A method of monitoring an immunotherapy treatment in a subject, the method comprising: administering to the subject an engineered T cell comprising a chimeric antigen receptor (CAR) and a nucleic acid molecule comprising a ligand binding domain; administering to the subject a radiolabeled tracer capable of binding to the ligand binding domain; and, detecting the level of radiolabeled tracer bound by imaging as a measure of the immunotherapy treatment.

### Embodiment 8

The method of any one of embodiments 5, 6 and 7, wherein the imaging is performed by positron emission tomography (PET), computerized tomography (CT) or bioluminescence (BL).

### Embodiment 9

A method of imaging engineered T cells in a subject, the method comprising: administering to the subject an engineered T cell comprising a chimeric antigen receptor (CAR) and a nucleic acid molecule comprising a ligand binding domain; administering to the subject a radiolabeled tracer capable of binding to the ligand binding domain; and, detecting the radiolabeled tracer by imaging using positron emission tomography (PET) or computed tomography (CT).

### Embodiment 10

The method of anyone of embodiments 5, 6, 7 and 9, wherein the ligand binding domain is E. coli dihydrofolate reductase (eDHFR).

### Embodiment 11

The method of any one of embodiments 5, 6, 7 and 9, wherein the radiolabeled tracer is [¹⁸F]fluoropropyl-trimethoprim ([¹⁸F]FPTMP).

### Embodiment 12

The method of any one of embodiments 5, 6, 7 and 9, wherein the engineered T cell(s) is/are autologous to the subject.

### Embodiment 13

The method of any one of embodiments 5, 6, 7 and 9, wherein the engineered T cell(s) is/are allogenic to the subject.

### Embodiment 14

The method of any one of embodiments 5, 6, 7 and 9, wherein the subject is a mammal.

### Embodiment 15

The method of embodiment 14, wherein the mammal is a human.

### Embodiments on Small Molecules for Dual Function PET and Cell Suicide Switches

### Embodiment 1

An engineered cell comprising a chimeric antigen receptor (CAR) and further comprising a nucleic acid molecule comprising a suicide gene comprising a ligand binding domain and a suicide domain wherein the ligand binding domain is capable of binding to radiolabeled tracer or a small molecule suicide switch.

### Embodiment 2

The engineered cell of embodiment 1, wherein the cell is a T cell.

### Embodiment 3

The engineered cell of embodiment 1, wherein the suicide domain is an inducible caspase 9 (iCasp9) domain.

### Embodiment 4

The engineered cell of embodiment 1, wherein the ligand binding domain is selected from the group consisting of FKBP, F36V-FKBP, and E. coli dihydrofolate reductase (eDHFR).

### Embodiment 5

The engineered cell of embodiment 3, wherein the iCasp9 domain comprises E. coli dihydrofolate reductase (eDHFR) ligand binding domain (eDHFR-iCasp9).

### Embodiment 6

The engineered cell of embodiment 5, wherein the eDHFR-iCasp9 further comprises a linker consisting of 15 or 18 amino acids in length.

### Embodiment 7

The engineered cell of embodiment 1, wherein the radiolabeled tracer is selected from the group consisting of [¹¹C]-Shield-1; [¹⁸F]-Shield-1; [¹¹C]-Trimethoprim ([¹¹C]-TMP) and [¹⁸F]-Trimethoprim ([¹⁸F]-TMP).

### Embodiment 8

A method of inducing apoptosis of an engineered cell, the method comprising introducing into the cell: a chimeric antigen receptor (CAR); a nucleic acid molecule comprising a suicide gene; and a ligand wherein the ligand comprises a dimerized Trimethoprim (TMP) that activates the suicide gene, thereby inducing apoptosis of the engineered cell.

### Embodiment 9

The method of embodiment 8, wherein the suicide gene comprises an inducible iCasp9 suicide domain comprising E. coli dihydrofolate reductase (eDHFR) ligand binding domain (eDHFR-iCasp9).

### Embodiment 10

The method of embodiment 9, wherein the eDHFR-iCasp9 further comprises a linker consisting of 15 or 18 amino acids in length.

### Embodiment 11

The method of embodiment 8, wherein the dimerized Trimethoprim is Bis-Trimethoprim (Bis-TMP) of the formula (I):

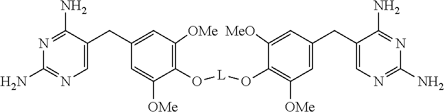

wherein L is a chemical linker having a length of about 1 to about 50 atoms.

### Embodiment 12

The method of embodiment 11, wherein the Bis-TMP is a compound selected from the group consisting of:

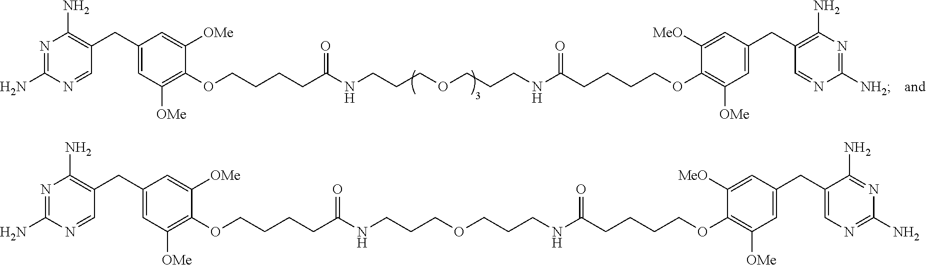

### Embodiment 13

A method of assessing the efficacy or toxicity of an adoptive cell therapy in a subject, the method comprising:

administering to the subject an engineered T cell comprising a chimeric antigen receptor (CAR) and a nucleic acid molecule comprising a suicide gene that comprises a ligand binding domain and a suicide domain; administering to the subject a radiolabeled tracer capable of binding to the ligand binding domain; detecting the amount of radiolabeled tracer bound by imaging; and, assessing the efficacy or toxicity of the adoptive cell therapy in the subject.

### Embodiment 14

The method of embodiment 13, wherein the ligand binding domain is selected from the group consisting of FKBP, F36V-FKBP, E. coli dihydrofolate reductase (eDHFR).

### Embodiment 15

The method of embodiment 13, wherein the radiolabeled tracer is selected from the group consisting of [¹¹C]-Shield-1; [¹⁸F]-Shield-1; [¹¹C]-Trimethoprim ([¹¹C]-TMP) and [¹⁸F]-Trimethoprim ([¹⁸F]-TMP).

### Embodiment 16

The method of embodiment 13, wherein when the adoptive cell therapy is assessed as toxic the radiolabeled tracer is replaced by a dimerized ligand capable of binding to the ligand binding domain and activating the suicide domain thereby inducing cell death of the engineered T cell.

### Embodiment 17

The method of embodiment 16, wherein the dimerized ligand comprises AP1903, AP20187 and Bis-TMP.

### Embodiment 18

A method of detecting the quantity of engineered T cells in a subject, the method comprising: administering to the subject an engineered T cell comprising a chimeric antigen receptor (CAR) and a nucleic acid molecule comprising a suicide gene that comprises a ligand binding domain and a suicide domain; administering to the subject a radiolabeled tracer capable of binding to the ligand binding domain; and, imaging the amount of radiolabeled tracer bound thereby detecting the quantity of engineered T cells in the subject.

### Embodiment 19

A method of monitoring an immunotherapy treatment in a subject, the method comprising: administering to the subject an engineered T cell comprising a chimeric antigen receptor (CAR) and a nucleic acid molecule comprising a suicide gene that comprises a ligand binding domain and a suicide domain; administering to the subject a radiolabeled tracer capable of binding to the ligand binding domain; and, detecting the level of radiolabeled tracer bound by imaging as a measure of the immunotherapy treatment.

### Embodiment 20

The method of any one of embodiments 13, 18 and 19, wherein the imaging is performed by positron emission tomography (PET).

### Embodiment 21

A method of imaging engineered T cells in a subject, the method comprising: administering to the subject an engineered T cell comprising a chimeric antigen receptor (CAR) and a nucleic acid molecule comprising a suicide gene that comprises a ligand binding domain and a suicide domain; administering to the subject a radiolabeled tracer capable of binding to the ligand binding domain; and, detecting the radiolabeled tracer by imaging using positron emission tomography (PET).

### Embodiment 22

The method of any one of embodiments 18, 19 and 21, wherein the suicide domain is an inducible caspase 9 (iCasp9).

### Embodiment 23

The method of any one of embodiments 18, 19 and 21, wherein the ligand binding domain is selected from the group consisting of FKBP, F36V-FKBP, E. coli dihydrofolate reductase (eDHFR).

### Embodiment 24

The method of any one of embodiments 18, 19 and 21, wherein the radiolabeled tracer is selected from the group consisting of [¹¹C]-Shield-1; [¹⁸F]-Shield-1; [¹¹C]-Trimethoprim ([¹¹C]-TMP) and [¹⁸F]-Trimethoprim ([¹⁸F]-TMP).

### Embodiment 25

The method of any one of embodiments 13, 18, 19 and 21, wherein the engineered T cell(s) is/are autologous to the subject.

### Embodiment 26

The method of any one of embodiments 13, 18, 19 and 21, wherein the engineered T cell(s) is/are allogenic to the subject.

### Embodiment 27

The method of any one of embodiments 13, 18, 19 and 21, wherein the subject is a mammal.

### Embodiment 28

The method of embodiment 27, wherein the mammal is a human.

### Embodiment 29

A composition comprising a chemical inducer of dimerization (CID), wherein the CID is a Bis-Trimethoprim (Bis-TMP) of the formula (I):

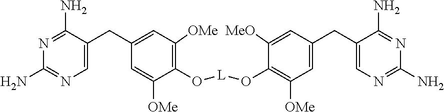

wherein L is a chemical linker having a length of about 1 to about 50 atoms.

### Embodiment 30

A composition comprising a ligand of F36V-FKBP of formula (II):

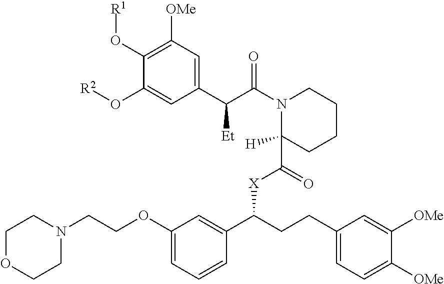

wherein

X is NH or O;

a) R¹ is CH₂Y, CH₂CH₂Y, or CH₂CH₂CH₂Y and R² is CH₃; or

b) R² is CH₂Y, CH₂CH₂Y, or CH₂CH₂CH₂Y and R¹ is CH₃; and

Y is F or ¹⁸F.

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C., pressure is at or near atmospheric.

### EXAMPLES

The present disclosure is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in these experiments are now described.

Cloning and Molecular Biology

pTRPE lentiviral vector encoding GD2-scFv-CD8 hinge 4-1BB-CD3z with E101K mutation was generated as described previously. ¹⁵ This CAR construct was subcloned into a pELPS lentiviral vector upstream of a T2A-mCherry gene to allow flow sorting of CAR⁺ T cells in these experiments To generate the eDFHR-YFP-2A-Renilla renformis luciferase (DYR) construct, PCR products encoding mammalian codon optimized eDHFR-YFP with a T2A cleavage site followed by Nhe-1 restriction site and renilla luciferase at the c-terminus were ligated into pELPS (coding sequence and protein sequence provided in the supplemental information).

Mammalian Cell Culture

HEK293 and HCT116 cells (American Type Cell Culture) were cultured in complete media: DMEM with 10% fetal bovine serum (Invitrogen), 2 mM glutamine, 100 U/mL penicillin and 100 mg/mL streptomycin (all from Gibco). Cells were maintained in a humidified incubator at 37° C. Yellow fluorescent protein-DHFR cells (“DHFR” cells) were made by introducing a YFP-DHFR fusion gene cloned into pBMN YFP-DHFR (addgene, plasmid #29326) used to generate amphotrophic retrovirus (Clonetech, #631505). HEK293 and HCT116 cells were incubated with retrovirus and polybrene (6 μg/mL) for 4 hrs at 37° C., passaged and selected with YFP fluorescence activated cell sorting (BD).

YFP-DHFR cells plated in a 6-well plate (1×10⁵ cells/well) were incubated overnight at 37° C. Live cell imaging was performed with fluorescence microscope using a GFP/YFP filter and phase contrast (Zeiss).

HCT116 cells (American Type Culture Collection) carrying the dhfr transgene were made as described previously.¹³ All cells (HCT116, 143b) were cultured in complete media: DMEM with 10% fetal bovine serum (Invitrogen), 2 mM glutamine, 100 U/mL penicillin and 100 mg/mL streptomycin (all from Gibco). Cells were maintained in a humidified incubator at 37° C.

CAR T Cell Generation

Human primary bulk T cells used in these experiments were collected from healthy volunteers and purified by the Human Immunology Core at the University of Pennsylvania. T cells were expanded by co-incubation with anti-CD3/anti-CD28-coated magnetic beads at a ratio of 3:1 beads to T cells. The next day, T cells were co-transduced with lentivirus encoding DYR and GD2-E101K CAR-T2A-mCherry (or singly transduced with DYR alone) at a multiplicity of infection of 5. Lentivirus had been harvested the supernatants of 293T cells transfected with the lentiviral plasmid along with packaging plasmids as described previously.²⁸ T cells were cultured in RPMI 1640 media supplemented with 10% FBS, 10 mmol/L HEPES buffer, 100 U/mL

penicillin, and 100 g/mL streptomycin sulfate, without additional cytokines until they had rested down with a cellular volume of approximately 400 fL. At that point, beads and media were removed, and the cells were washed in Dulbecco's phosphate-buffered saline (PBS), and resuspended in fetal bovine serum (FBS) with 5% dimethylsulfoxide (DMSO) for cryopreservation.

In Vitro Assays

Mammalian Cellular Uptake Studies.

HCT116 dhfr cells were plated in a 96-well plate (40K cells/well) 24 h prior to assay, incubated with [¹⁸F]FPTMP (˜70,000 cpm/50 uL/well, saline, <1% ethanol) in Opti-Mem (Gibco) for 120 minutes at 37° C. Excess unlabeled TMP (10 μM) or methotrexate (MTX, 10 μM) were added as blocking agents at the same time as adding the radiotracer, to determine nonspecific binding. Protein was quantified using the Lowry method and uptake assayed on a gamma counter (Perkin Elmer). [¹⁸F]FPTMP uptake in primary T cells was performed by incubating 1 million CAR and DYR T cells in solution in a 1.7 mL epitube with (2M CPM/50 μL) for 30 minutes, washed 3× with cold PBS, and assayed with a gamma counter. Uptake was measured by dividing counts by incubated dose of [¹⁸F]FPTMP and normalizing to either protein concentration (% ID/μg) or cell number (% ID/million cells).

T Cell Bioluminescence Experiment.

Primary human T cells (untransduced) and DYR T-cells (single transduction) that were sorted on YFP were added to a 96 well plate (100,000 cells). Coelenterazine-h (Nanolight) was added to the wells to a final concentration 1.5 μM and cells were assayed on a luminometer (Perkin Elmer).

Flow Cytometry and Cell Sorting

For sorting, T cells that had been transduced with the DYR and CAR viruses, or DYR alone were cultured for 8 days prior to sorting. Cells were washed with sorting buffer (PBS with 2% BSA and 1% HEPES) and analyzed and sorted on a FACS Aria II (BD) at the University of Pennsylvania Flow Cytometry and Cell Sorting. Double positive (YFP+mCherry+) or single positive (YFP+) cells were collected and returned to conditioned media for the remainder of culture. For staining of cell lines, trypsinized, PBS-washed tumor line cells were incubated with APC-conjugated anti-GD2 clone 14G2a (BioLegend, cat no. 357306) or isotype control at 1:30 dilution for 15 minutes at room temperature. Cells were then washed once in 2 mL PBS and analyzed on an LSRFortessa (BD).

Mouse Models

Tumor Uptake.

CD1 nu/nu female mice (Charles River, 6-8 weeks, n=3) received subcutaneous dorsal, shoulder injections of 10×10⁶ HCT116 control or eDHFR cells. After 21 days growth, tumors were palpable and animals were anesthetized (2% isoflurane), placed on the warmed stage for small animal PET and micro CT imaging (Molecubes), and given a tail vein injection of [¹⁸F]FPTMP (˜100 μCi/mouse). Dynamic PET/CT scans were initiated at the time of injection and acquired over 45 minutes. Static PET/CT scans were acquired for 30 minutes at 3 h and 6 h post injection. Elliptical regions of interest (ROI's) were drawn around each tumor using the CT images, and the maximum counts from each ROI was determined using MIM (MIM Software Inc., Cleveland Ohio). The ratio of uptake between eDHFR tumors and control tumors was calculated for various time points post injection. At the end of the imaging session mice were sacrificed and tissue were taken for ex vivo biodistribution calculation. Uptake in percent injected dose per gram (% ID/g) was assayed with a gamma counter (Perkin Elmer).

CAR T Cells Tracking.

Female NOD-SCID-Il2rg^{-/-} (NSG) mice (Jackson Labs) were subcutaneously xenografted with HCT116 human colon cancer cells (ATCC, 10×E6) on the left shoulder of four animals per group. 143b human osteosarcoma (ATCC, 10×E6) were implanted contralateral side of the animal in 100 μL PBS (Corning). The tumors were allowed to grow for 14 days and then animals were given either 1 million DYR T cells or 1 million CAR+DYR T cells by tail vein injection in 100 μL PBS (n=4 per group). On day 7 and 13 after T cell injection, all mice were administered “Inject-A-Lume” highly pure Coelenterazine (Nanolight, 100 μg/mouse in 30 μL of solvent (Fuel-Inject). Mice were imaged for 1-3 minutes dorsally and ventrally using an IVIS spectrum (Perkin Elmer) under isoflurane anesthesia. On the same days, all mice were then injected with [¹⁸F]FPTMP IV (˜100 μCi/mouse) and imaged with PET/CT 3 h after tracer administration and data was analyzed as above. On day 13, mice were sacrificed and tissues harvested for ex vivo autoradiography and immunohistochemistry. Mouse number 1 of the CART cell group died during PET/CT imaging during day 13 and was omitted from analysis. All animal studies were completed with University of Pennsylvania's Institutional Animal Care and Use Committee, IACUC approval.

Autoradiography and Tissue Histology.

Mice were sacrificed after [¹⁸F]FPTMP injection and imaging. Tissues were dissected and embedded with in OCT. Sections (10 micron) were cut and exposed to a phosphor-plate overnight (GE) and developed on a Typhoon digital autoradiograph (GE). Anti-human CD8 IHC and H&E staining of contiguous frozen sections was performed at the University of Pennsylvania Perelman School of Medicine Pathology Clinical Service Center and digitized (Zeiss Axio System). Pathology images presented here taken using screen captures from QuPath (Open Source Digital Pathology, github). Auto-sensing of DAB staining for anti-human CD8 was used using “simple tissue detection” and “fast cell counting” features using QuPath.

Chemical Synthesis

[¹⁸F]FPTMP synthesis was performed as previous described.¹⁴

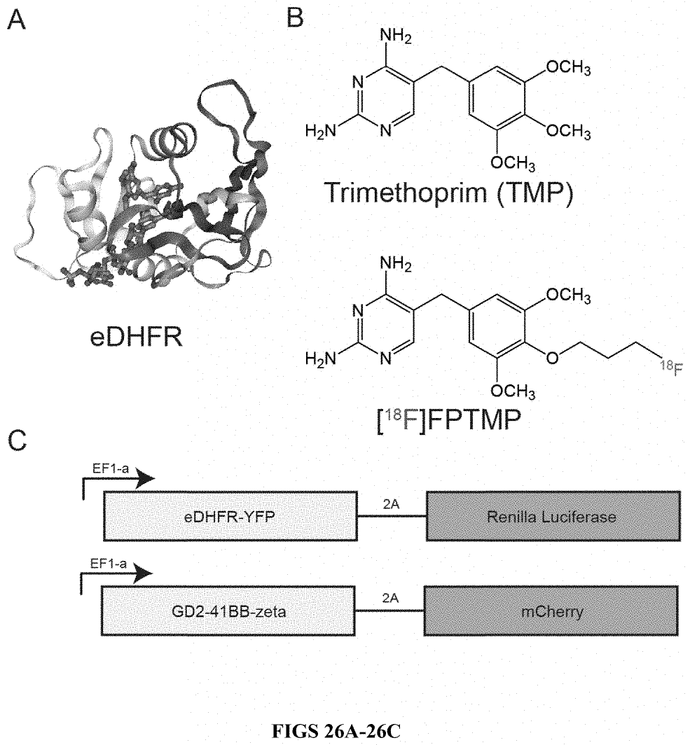

To address the clear need for facile human imaging of genetically engineered cells such as chimeric antigen receptor (CAR) T cells and to expand the tools available to investigators, the development of a high sensitivity PET reporter gene (E. coli dihydrofolate reductase, eDHFR) is shown paired with a small molecule PET probe, [¹⁸F]fluoropropyl-trimethoprim [¹⁸F]FPTMP (FIGS. 26A-26B).^{13,14} Previous investigations have shown that [¹¹C]TMP paired with eDFHR had promising uptake in tumor xenograft models. Disclosed herein are in vitro uptake and in vivo rodent models to characterize the sensitivity and biodistribution of the reporter-probe system with [¹⁸F]FPTMP and then this strategy was applied to CAR T cells targeted to the GD2 disialoganglioside, a tumor antigen present on several cancers, such as neuroblastoma, melanoma, and some types of osteosarcoma (FIG. 26C).¹⁵ In NSG mice bearing a GD2+ human osteosarcoma xenograft at one shoulder and GD2− colon cancer on the other shoulder, the CAR T cells initially accumulate in the spleen, followed by trafficking to antigen positive tumors as compared to control tumors using bioluminescence and PET imaging. Key aspects in this work are the whole-animal PET images, which suggest a high degree of signal to noise originating from foci of CAR T cells invading into a tumor, and autoradiography correlation with CD8 immunohistochemistry. PET detectable engineered cells are estimated to number in the thousands per mm³. This imaging sensitivity of [¹⁸F]FPTMP-eDHFR suggests significant potential for future clinical application and broadening the available tools for human gene/cell therapy protocols.

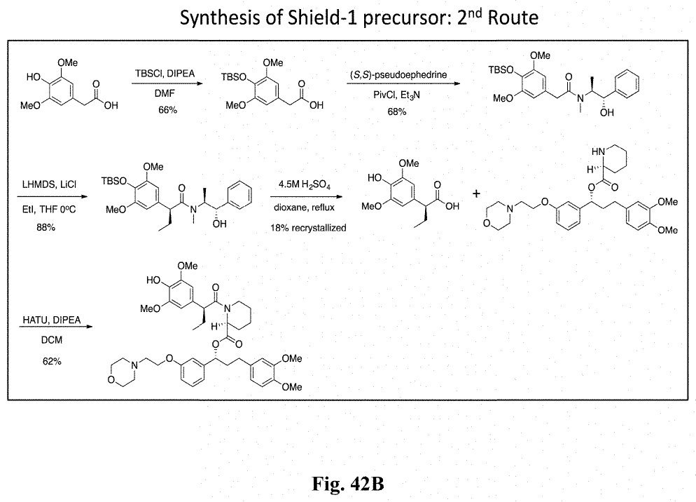

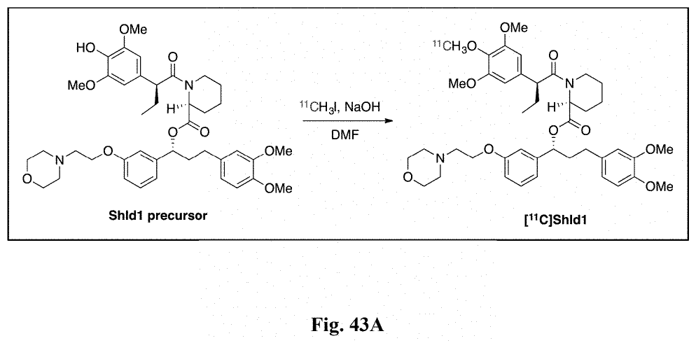

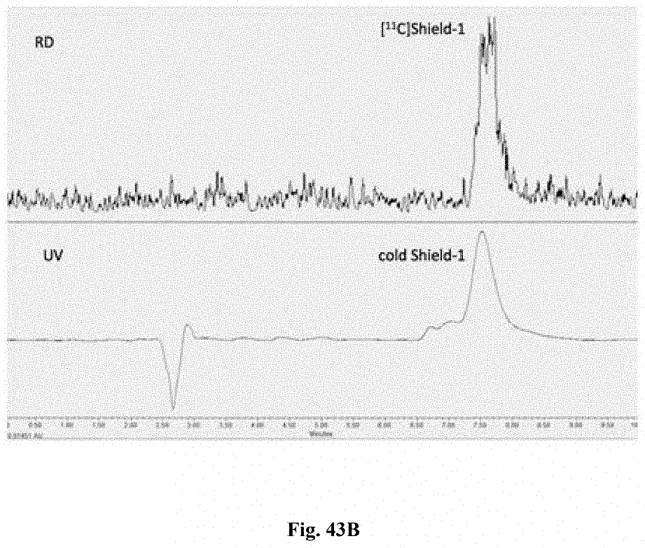

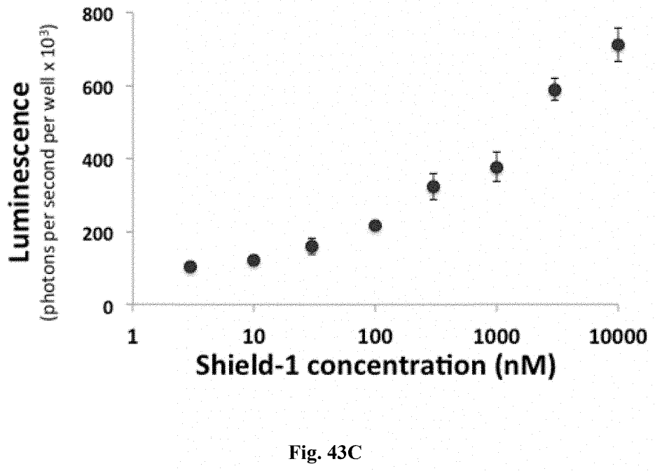

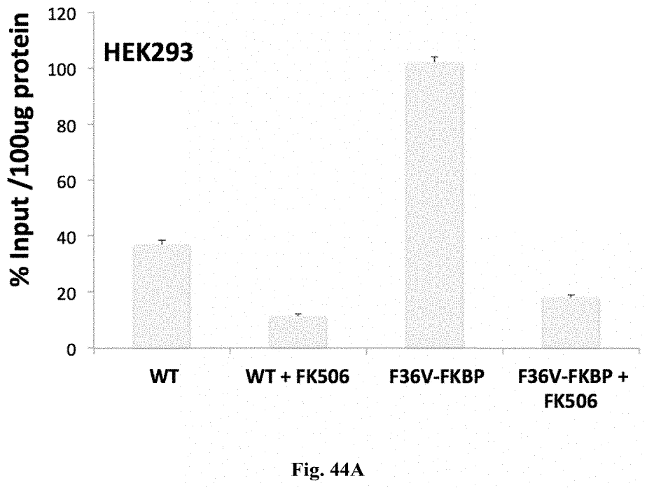

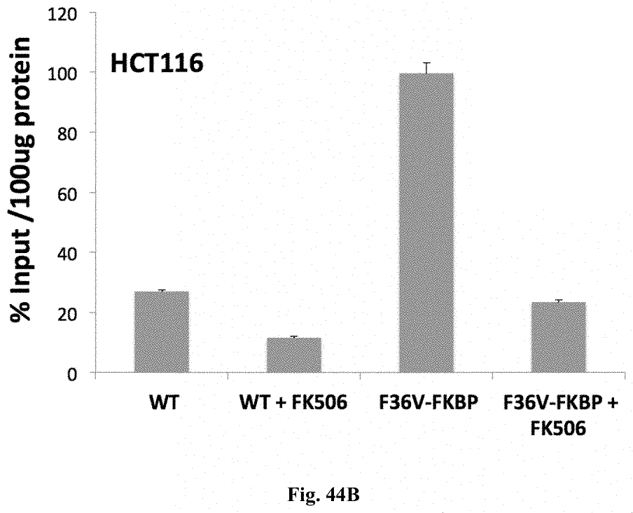

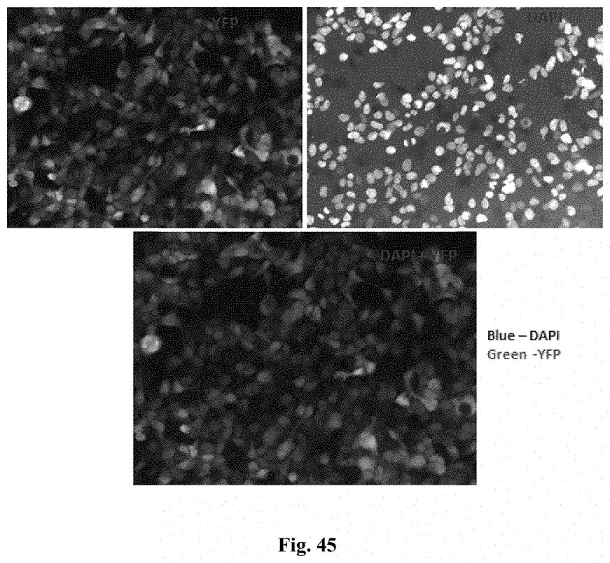

Shield-1 Precursor Synthesis (FIG. 42B):

To a solution of (S)-2-(4-hydroxy-3,5-dimethoxyphenyl)butanoic acid (79.2 mg, 0.15 mmol) in 1.5 mL dichloromethane was added N,N-diisopropylethylamine (107.7 μL, 0.61 mmol) and HATU (76.5 mg, 0.20 mmol). A solution of the amine ((R)-3-(3,4-dimethoxyphenyl)-1-(3-(2-morpholinoethoxy)phenyl)propyl (S)-piperidine-2-carboxylate) in dichloromethane was then added, and the reaction mixture was stirred at room temperature under nitrogen atmosphere for 24 h. The reaction was monitored by TLC and upon completion of reaction, the reaction mixture was quenched with saturated aqueous NaHCO₃ and extracted with dichloromethane (10 mL×3). The combined organic layers were dried over Na₂SO₄ and the solvent was evaporated under reduced pressure to obtain crude product. The crude material was purified by column chromatography on silica gel (5% methanol-dichloromethane) to obtained pure Shield-1 precursor. Yield 62% (69.8 mg); silica gel TLC R_{f}=0.33 (5% methanol-dichloromethane).

[¹¹C]Shield-1 Radiosynthesis (FIG. 43A):

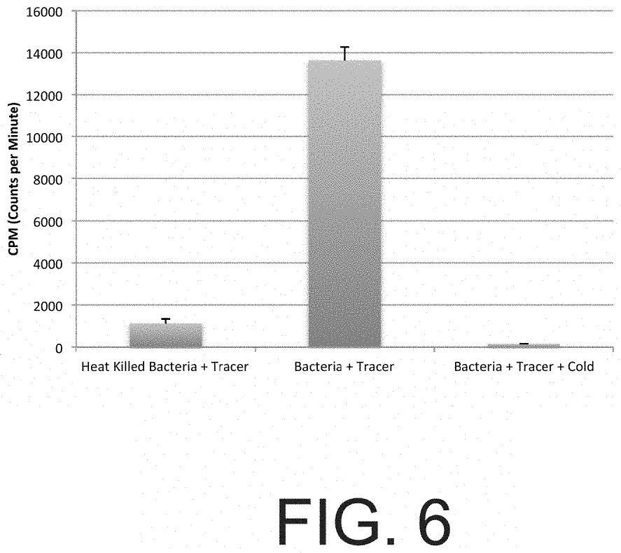

The [¹¹C]CO₂ produced by the IBA Cyclone cyclotron is converted to [¹¹C]CH₃I utilizing the GE PETtrace Me Microlab system. Briefly, [¹¹C]CO₂ is transformed to [¹¹C]CH₄ using a nickel catalyst in the presence of hydrogen gas and the produced [¹¹C]CH₄ is then reacted with gaseous iodine at high temperature to form [¹¹C]CH₃I. The radiochemical synthesis of [¹¹C]Shield1 was carried out by methylating the Shield-1 precursor. The produced [¹¹C]CH₃I was bubbled to the reaction vial containing 1.0 mg Shield-1 precursor in 200 μL anhydrous DMF and 4 μL of 5N NaOH aqueous solution. The reaction mixture was then heated at 85° C. for 5 min, quenched with 1.5 mL of HPLC mobile phase and injected to a reversed-phase Phenomenex Luna C18 (250×10 mm) HPLC column for purification using 45:55 Acetonitrile/Water with 0.1% TFA as HPLC mobile phase at a flow rate of 5 mL/min. [¹¹C]Shield1 (Rt=15 min) was collected and diluted onto a vial containing 30 mL water, passed through a Sep-Pak C18 cartridge (Waters Corp.) and washed with 10 mL water. The product was then eluted with 1 mL of 200 proof ethanol followed by dilution with 9 mL phosphate buffered saline (PBS). The radiochemical yield is 5% with a radiochemical purity greater than 95% and a specific activity greater than 533 GBq/umoL.

[¹¹C]Shield-1 Cell Uptake Study (FIGS. 6A-6B)
Radioligand: [¹¹C]-Shield-1 in PBS
Cell line: HCT 116-F36V-FKBP and HCT116; HEK 293-F36V-FKBP and HEK293
Input cpm: ˜240,000
Incubation media: Optimem (no FBS)+PBS (with Ca²⁺& Mg²⁺) 1:1 (500 uL total volume)
Blocking agent: 10 uM FK506 in optimum
Plate 1×10⁷ cells/well (6-well) 24 hours prior to uptake experiment
Trypsinize and divide cells in 2 eppendorf tube per well
Incubate with [¹¹C]-Shield-1 with or without blocking agent at 37° Celsius for 40 min (500 uL)
At end of incubation period, spin cells, wash 2× with 900 μL PBS
Protein assay-Lowry method were carried out to normalize results

Western Blot Procedure:

Cell Lysis

Cells from T75 flask were harvested and lysed with 0.5 mL of lysis buffer (RIPA buffer+phosphatase inhibitor cocktail 2 & 3+protease inhibitor). Incubate on ice for 30 min, sonicate and spin at 12.00 rpm for 10 min.

Protein Determination by Lowry Method—Run Protein Gel
Gel: TGX Miniprotean precast gels, 4-20% polyacrylamide gel, 10 well-50 uL, 8.6×6.7 cm (W×L) Cat #4561094
Running buffer: 1×Tris/Glycine/SDS
System: BioRad Tetra cell, 100 V, 1 hr 10 min
Ladder: Biorad Precison plus Protein dual color standard
Amount of protein loaded: 20 ug (1:1 with Laemmli solution)
Transfer to PVDF membrane
PVDF: Transblot Turbo Mini PVDF transfer pack (Cat #1704156)
System: BioRad Transblot Turbo (Mixed MW, 7 min)
Blocking: Odyssey blocking buffer in PBS (P/N 927-40000), 1 hr at room temp
Wash in TBST (1×TBS buffer+0.1% Tween 20)
Incubate in primary Antibody: Anti-HA (12CA5), 0.4 mg/mL in PBS, dilution used 1:1,000 mouse monoclonal: Anti-GapDH, 0.2 mg/mL, dilution used 1:1,000; goat polyclonal in TBST with 5% blocking buffer, overnight at 4° C. with gentle shaking
Wash 4× with TBST, 10 min each with gentle shaking
Incubate in secondary antibody: Goat anti-mouse IgG, dilution used 1:10,000, 800 CW-Licor: Donkey anti-goat IgG, dilution used 1:10,000, 680 RD-Licor in TBST with 5% blocking buffer, 1 hr at room temp with gentle shaking
Wash 4× with TBST, 10 min each with gentle shaking
Read Odyssey CLx Imaging system Li-COR Biosciences

Labeling of DHFR-icasp9 Cells with Ligandlink (Fluorescein Label with TMP)
Day 1 plate cells on glass bottom dish 35 mm

| | |
|---|---|
| HCT116 DHFR-icasp9 | 1.5 × 10⁶ |
| HEK293 | 1.5 × 10⁶ |
| MB231-luc | 2.0 × 10⁶ |

Day 2 labeling of cells with ligand link
Add 30 uL DMF:acetic acid to 1 vial ligand link (1 mM)
Dilute to 2 uM in DMEM with 10% FBS
Aspirate media off dish and add 1 mL diluted ligand link label
Incubate for 2 hrs
Wash with PBS (2×)
Add PBS with 10 mM glucose
View fluorescence microscope (Zeiss)

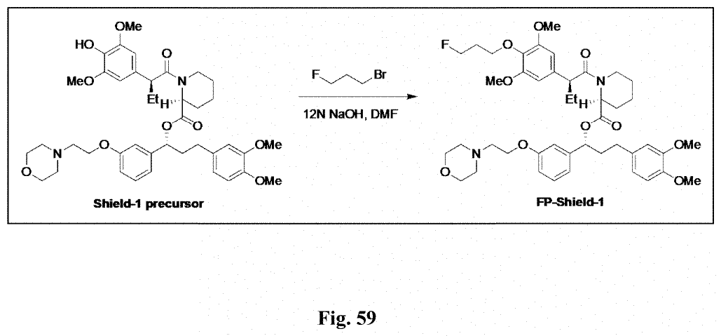

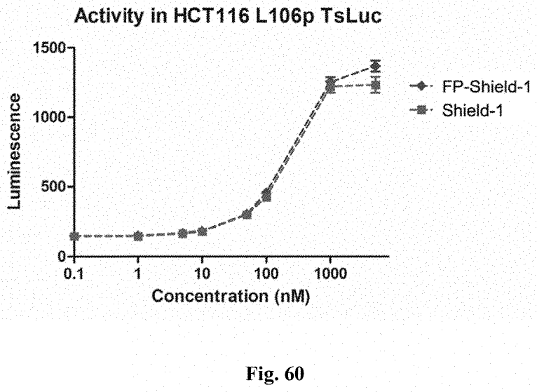

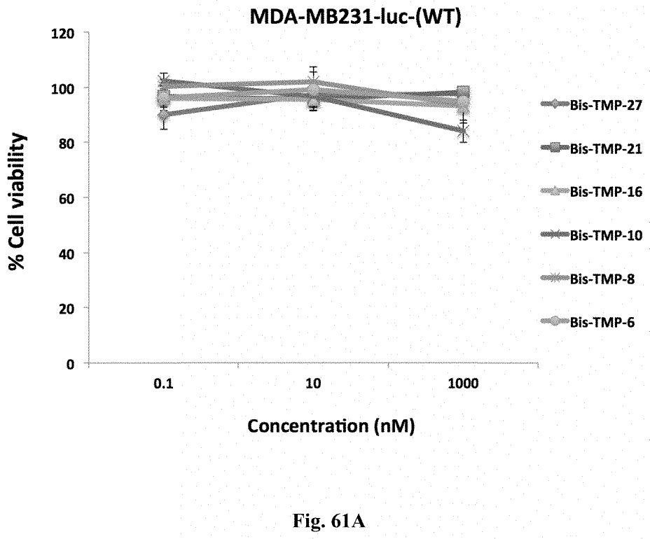

FP-Shield-1 Synthesis (FIG. 59):

To a solution of Shield-1 precursor (5.0 mg, 6.8 nmols) in DMF (0.2 mL) was added 12 N NaOH (5 μL) and 1-bromo-3-fluoropropane (3.79 mg, 27.2 nmols). The reaction mixture heated to 80° C. for 15 mins, and monitored by LC-MS. Upon completion of reaction, the crude material was purified by IPLC using reverse phase C-18 silica gel column (40% acetonitrile-water in 0.1% trifluoroacetic acid) to obtain pure FP-Shield-1. Yield 28% (1.5 mg).

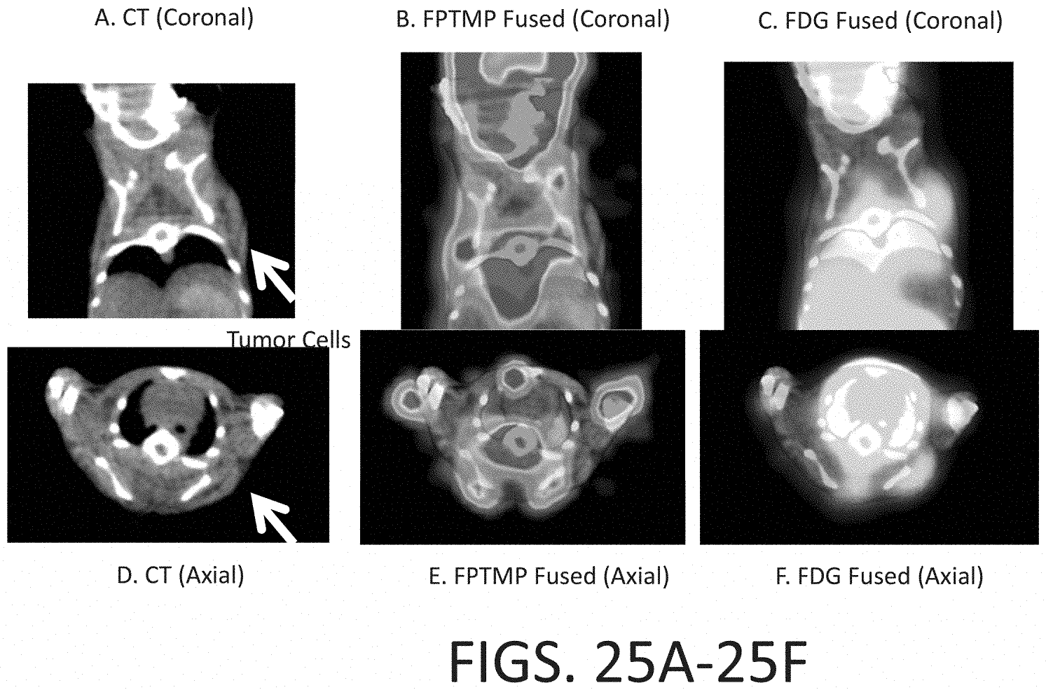

### Synthesis of the TMP Library (FIG. 25)

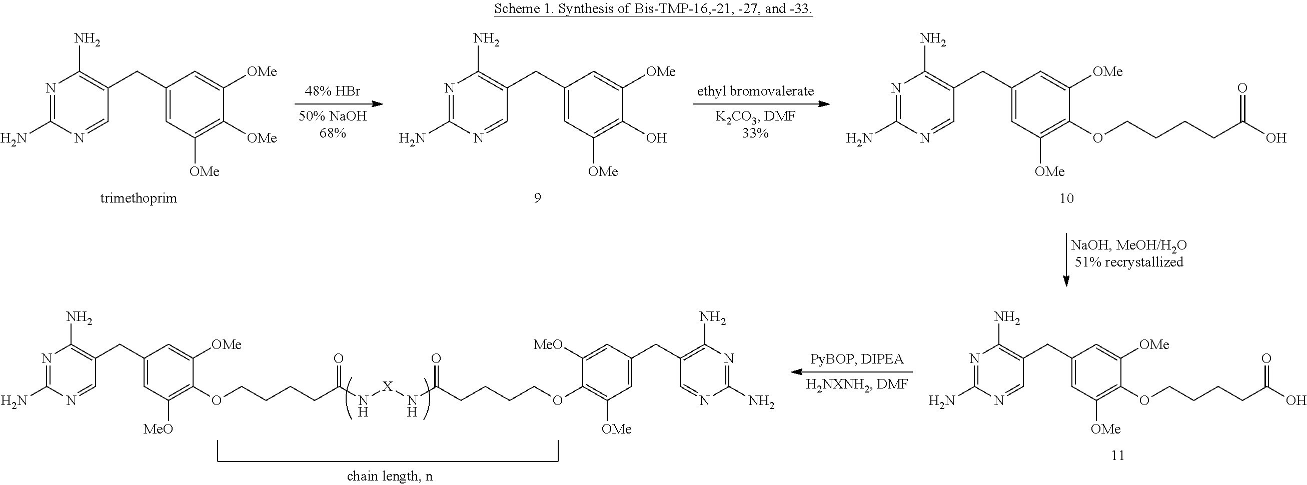

### Ethyl 5-(4-((2,4-diaminopyrimidin-5-yl)methyl)-2,6-dimethoxyphenoxy)pentanoate (10)

DMSO (16 mL) was added to a degassed flask containing phenol 9 (2.20 g, 7.97 mmols, 1 equiv). The resulting solution was evacuated then purged 3 times with nitrogen gas. DBU (1.31 mL, 8.76 mmol, 1.1 equiv) and ethyl-1-5-bromovalerate (1.39 mL, 8.76 mmol, 1.1 equiv) were added sequentially and the final mixture was evacuated then purged 3 time with nitrogen gas. After 2 h, the mixture was partially concentrated via rotavap and the resulting concentrated solution was loaded onto a silica gel column and eluted with 2-10% methanol in dichloromethane to provide 1.27 g (39% yield) of a white solid. HRMS calcd: 405.2138, obsvd: 405.2138

### 5-(4-((2,4-diaminopyrimidin-5-yl)methyl)-2,6-dimethoxyphenoxy)pentanoic Acid (11)

To a solution of ester 10 (652 mg, 1.61 mmol, 1 equiv) in methanol (16 mL, 0.1 M) was added aqueous sodium hydroxide solution (1 M, 4.8 mL, 4.8 mmol, 3 equiv). After 2.5 h, methanol was removed via rotary evaporation and the resulting aqueous solution was neutralized to pH7 using an aqueous hydrochloric acid solution (1 M, ca. 4.5 mL) which resulted in precipitation of a white solid. The solids were washed with cold water then dried to provide the desired acid (308.6 mg, 51% yield). HRMS: calcd: 377.1825, obser: 377.1833

### N,N′-(Ethane-1,2-diyl)bis(5-(4-((2,4-diaminopyrimidin-5-yl)methyl)-2,6-dimethoxyphenoxy)pentanamide) (Bis-TMP-16)

Acid 11 (42.7 mg, 0.113 mmols, 2.5 equiv) and benzotriazol-1-yl-oxytripyrrolidinophosphonium hexafluoroposphate (PyBOP, 944 mg, 0.18 mmol, 4 equiv) were combined and evacuated then purged with nitrogen gas. DMF (1 mL) and DIPEA (47 μL, 0.27 mmol, 6 equiv) were added and allowed to stir for 1.5 h. 1,2-Diaminoethane (3 μL, 0.045 mmol, 1 equiv) was added and the resulting solution was allowed to stir overnight. The crude sample was purified by prep-HPLC to afford the desired product Bis-TMP-16 (22.5 mg, 16% yield) as a clear oil. HRMS: calcd: 777.4048, observ: 777.4028

### N,N′-(oxybis(propane-3,1-diyl))bis(5-(4-((2,4-diaminopyrimidin-5-yl)methyl)-2,6-dimethoxyphenoxy)pentanamide) (Bis-TMP-21)

Acid 11 (59.0 mg, 0.1157 mmols, 2.5 equiv) and benzotriazol-1-yl-oxytripyrrolidinophosphonium hexafluorophosphate (PyBOP, 130.5 mg, 0.251 mmol, 4 equiv) were combined and evacuated then purged with nitrogen gas. DMF (1 mL) and DIPEA (66 μL, 0.376 mmol, 6 equiv) were added and allowed to stir for 30 min. Bis(3-aminopropyl) ether (8.3 mg, 0.063 mmol, 1 equiv) was added and the resulting solution was allowed to stir overnight. The crude sample was purified by prep-HPLC to afford the desired product Bis-TMP-21 (55.7 mg, 41% yield) as a clear oil. HRMS: calcd: 849.4614, observ: 849.4623

### N,N′-(((oxybis(ethane-2,1-diyl))bis(oxy))bis(propane-3,1-diyl))bis(5-(4-((2,4-diaminopyrimidin-5-yl)methyl)-2,6-dimethoxyphenoxy)pentanamide) (Bis-TMP-27)

Acid 11(41.9 mg, 0.11 mmols, 2.5 equiv) and benzotriazol-1-yl-oxytripyrrolidinophosphonium hexafluorophosphate (PyBOP, 93 mg, 0.178 mmol, 4 equiv) were combined and evacuated then purged with nitrogen gas. DMF (1 mL) and DIPEA (46 μL, 0.267 mmol, 6 equiv) were added and allowed to stir for 30 min. 4,7,10-Trioxa-1,13-tridecanediamine (9.8 μL, 0.045 mmol, 1 equiv) was added and the resulting solution was allowed to stir overnight. The crude sample was purified by prep-HPLC to afford the desired product Bis-TMP-27 (18.8 mg, 30% yield) as a clear oil. HRMS [M+Na]: calcd: 959.4967, observ: 959.4959

### N,N′-(4,7,10,13,16-pentaoxanonadecane-1,19-diyl)bis(5-(4-((2,4-diaminopyrimidin-5-yl)methyl)-2,6-dimethoxyphenoxy)pentanamide) (Bis-TMP-33)

Acid 11 (60.3 mg, 0.16 mmols, 2.5 equiv) and benzotriazol-1-yl-oxytripyrrolidinophosphonium hexafluorophosphate (PyBOP, 133 mg, 0.26 mmol, 4 equiv) were combined and evacuated then purged with nitrogen gas. DMF (1 mL) and DIPEA (67 μL, 0.384 mmol, 6 equiv) were added and allowed to stir for 30 min. 1,19-Diamino-4,7,10,13,16-pentaoxanonadecane (19.8 mg, 0.064 mmol, 1 equiv) was added and the resulting solution was allowed to stir overnight. The crude sample was purified by prep-HPLC to afford the desired product Bis-TMP-33.

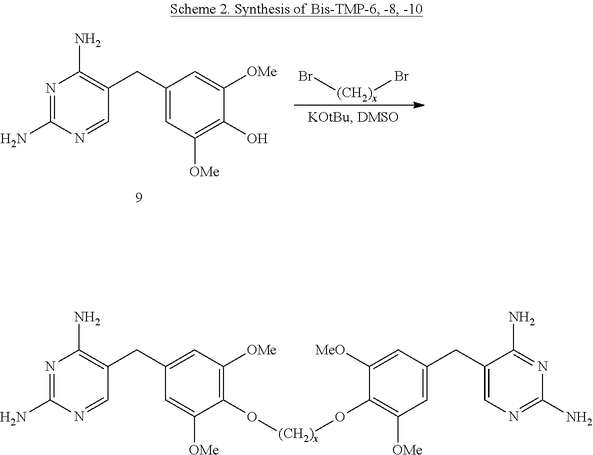

### 5,5′-(((butane-1,4-diylbis(oxy))bis(3,5-dimethoxy-4,1-phenylene))bis(methylene))bis(pyrimidine-2,4-diamine) (Bis-TMP-6)

DMSO (5.0 mL) was added to a degassed flask containing phenol 9 (200 mg, 0.724 mmols, 1.0 equiv). The resulting solution was evacuated then purged 3 times with nitrogen gas. Potassium tert-butoxide (97.5 mg, 0.87 mmol, 1.2 equiv) and 1,4-dibromobutane (28.2 μL, 0.23 mmol, 0.33 equiv) were added sequentially and the final mixture was evacuated then purged 3 time with nitrogen gas. The solution was stirred at room temperature (rt) for 48 h, and the mixture was partially concentrated under reduced pressure and the resulting concentrated liquid was purified by column chromatography on reverse phase C-18 silica gel (acetonitrile-water) to obtain Bis-TMP-6. Yield 21% (93.1 mg).

### 5,5′-(((hexane-1,6-diylbis(oxy))bis(3,5-dimethoxy-4,1-phenylene))bis(methylene))bis(pyrimidine-2,4-diamine) (Bis-TMP-8)

DMSO (5.0 mL) was added to a degassed flask containing phenol 9 (500 mg, 1.81 mmols, 1 equiv). The resulting solution was evacuated then purged 3 times with nitrogen gas. Potassium tert-butoxide (243 mg, 2.17 mmol, 1.2 equiv) and 1,6-dibromohexane (90 μL, 0.59 mmol, 0.33 equiv) were added sequentially and the final mixture was evacuated then purged 3 time with nitrogen gas. The solution was stirred at room temperature (rt) for 48 h, and the mixture was partially concentrated under reduced pressure and the resulting concentrated liquid was purified by column chromatography on reverse phase C-18 silica gel (acetonitrile-water) to obtain Bis-TMP-8. Yield 19% (220 mg).

### 5,5′-(((octane-1,8-diylbis(oxy))bis(3,5-dimethoxy-4,1-phenylene))bis(methylene))bis(pyrimidine-2,4-diamine) (Bis-TMP-10)

DMSO (2.0 mL) was added to a degassed flask containing phenol 9 (200 mg, 0.72 mmols, 1 equiv). The resulting solution was evacuated then purged 3 times with nitrogen gas. Potassium tert-butoxide (97 mg, 0.86 mmol, 1.2 equiv) and 1,8-dibromooctane (44 μL, 0.23 mmol, 0.33 equiv) were added sequentially and the final mixture was evacuated then purged 3 time with nitrogen gas. The solution was stirred at rt for 48 h, and the mixture was partially concentrated under reduced pressure and the resulting concentrated liquid was purified by column chromatography on reverse phase C-18 silica gel (acetonitrile-water) to obtain Bis-TMP-10. Yield 17% (84.2 mg).

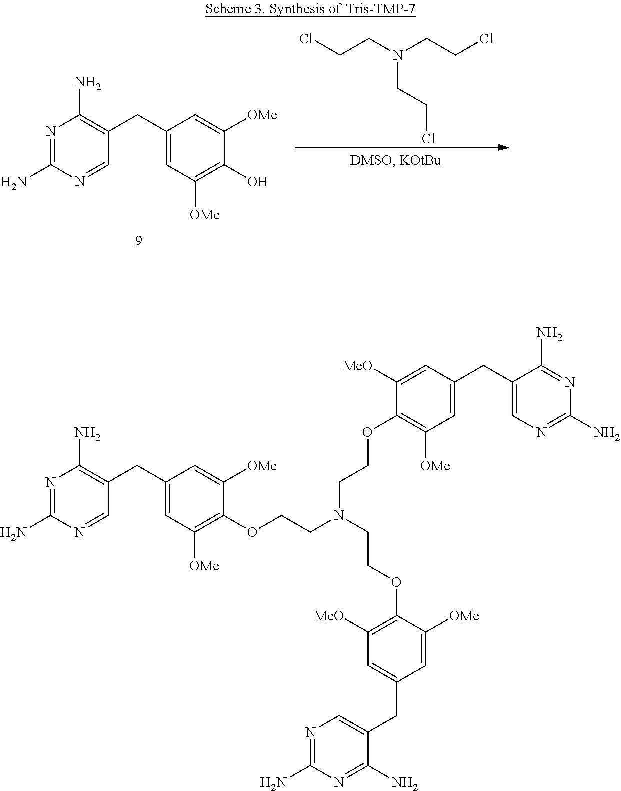 [SMILES: COc1cc(Cc2cnc(N)nc2N)cc(OC)c1O ; ClCCN(CCCl)CCCl ; COc1cc(Cc2cnc(N)nc2N)cc(OC)c1OCCN(CCOc1c(OC)cc(Cc2cnc(N)nc2N)cc1OC)CCOc1c(OC)cc(Cc2cnc(N)nc2N)cc1OC]

### 5-(4-(2-(bis(2-(4-((2,4-diaminopyrimidin-5-yl)methyl)-2,6-dimethoxyphenoxy)ethyl)amino)ethoxy)-3,5-dimethoxybenzyl)pyrimidine-2,4-diamine (Tris-TMP-7)

DMSO (2.0 mL) was added to a degassed flask containing phenol 9 (229 mg, 0.82 mmols, 1 equiv). The resulting solution was evacuated then purged 3 times with nitrogen gas. Potassium tert-butoxide (112 mg, 0.93 mmol, 4.0 equiv) and tris(2-chloroethyl)amine (50 μL, 0.21 mmol, 0.25 equiv) were added sequentially and the final mixture was evacuated then purged 3 times with nitrogen gas. The solution was stirred at rt for 48 h, and the mixture was partially concentrated under reduced pressure and the resulting concentrated liquid was purified by column chromatography on reverse phase C-18 silica gel (acetonitrile-water) to obtain Tris-TMP-7. Yield 7% (48.1 mg).

Procedure for DHFR-iCasp9 Cell Kill Assay with Bis-TMP Compounds:

Cell culture: Cell lines (MDA-MB231-luc transduced with DHFR-icasp9 with different linker lengths) were cultured in DMEM (Gibco) with 10% fetal bovine serum (Sigma), 100 U/mL penicillin and 100 ug/mL streptomycin (Invitrogen). Incubation is done at 37° C. with 5% CO2

Cell assay: Cells were plated on 96 well black plate, 3×10⁴ cells/well. The next day, cells were treated with varying concentrations of Bis-TMP compounds (Bis-TMP-#). Cells were incubated for 24 hours and luciferin was added (120 ug/mL), after which bioluminescence was measured using Perkin Elmer Enspire Multi-mode Plate reader.

Luminescence Assay with HCT116-L106P-tsLuc Cells Following Treatment with Shield-1 Compounds:
Day 1: Plate 50,000 cells/50 uL/well on 96 well black plate.
Day 2: Add compounds (FP-Shield-1, control Shield-1 in 50 uL volume). Incubate for 24 h at 37° C. with CO₂.
Day 3: Add luciferin and read immediately.

Sequences
DYR Gene Sequence (SEQ ID NO: 1):
Ggatcc(BamHI)ATGATAAGTTTGATTGCTGCTCTGGCTGTGGACCGGGTAATCGG
TATGGAAAACGCCATGCCCTGGAACCTGCCTGCCGATTTGGCTTGGTTCAAGCGCAATAC
CCTGAACAAACCAGTAATCATGGGAAGGCATACATGGGAAAGCATTGGAAGACCACTTCCC
GGTAGAAAGAATATTATCCTGTCTAGCCAGCCCGGCACGGATGATAGGGTGACATGGGTA
AAGAGCGTCGATGAGGCGATTGCGGCGTGTGGTGACGTGCCGGAAATTATGGTTATCGGA
GGCGGCAGGGTCTACGAACAGTTCCTGCCGAAGGCACAGAAGCTGTACCTCACCCACATC
GATGCAGAGGTGGAAGGAGACACGCACTTTCCAGATTACGAGCCTGATGACTGGGAGAGT
GTTTTTAGCGAATTCCATGACGCAGACGCCCAAAACTCTCACTCCTACTGCTTTGAGATTCT
CGAACGAAGGgcatgcgtgagcaagggcgaggagctgttcaccggggtggtgcccatcctggtcgagctggacggcgacgt
aaacggccacaagttcagcgtgtccggcgagggcgagggcgatgccacctacggcaagctgaccctgaagttcatctgcaccacc
ggcaagctgcccgtgccctggcccaccctcgtgaccaccttcggctacggcctgcagtgcttcgcccgctaccccgaccacatgaag
cagcacgacttcttcaagtccgccatgcccgaaggctacgtccaggagcgcaccatcttcttcaaggacgacggcaactacaagac
ccgcgccgaggtgaagttcgagggcgacaccctggtgaaccgcatcgagctgaagggcatcgacttcaaggaggacggcaacat
cctggggcacaagctggagtacaactacaacagccacaacgtctatatcatggccgacaagcagaagaacggcatcaaggtgaa
cttcaagatccgccacaacatcgaggacggcagcgtgcagctcgccgaccactaccagcagaacacccccatcggcgacggccc
cgtgctgctgcccgacaaccactacctgagctaccagtccgccctgagcaaagaccccaacgagaagcgcgatcacatggtcctg
ctggagttcgtgaccgccgccgggatcactctcggcatggacgagctgtacaagGGCAGCGGAGAGGGCAGAGGAA
GTCTTCTAACATGCGGTGACGTGGAGGAGAATCCCGGCCCTgctagcacttcgaaagtttatgatccaga
acaaaggaaacggatgataactggtccgcagtggtgggccagatgtaaacaaatgaatgttcttgattcatttattaattattatgattca
gaaaaacatgcagaaaatgctgttatttttttacatggtaacgcggcctcttcttatttatggcgacatgttgtgccacatattgagccagta
gcgcggtgtattataccagaccttattggtatgggcaaatcaggcaaatctggtaatggttcttataggttacttgatcattacaaatatctta
ctgcatggtttgaacttcttaatttaccaaagaagatcatttttgtcggccatgattggggtgcttgtttggcatttcattatagctatgagcatc
aagataagatcaaagcaatagttcacgctgaaagtgtagtagatgtgattgaatcatgggatgaatggcctgatattgaagaagatatt
gcgttgatcaaatctgaagaaggagaaaaaatggttttggagaataacttcttcgtggaaaccatgttgccatcaaaaatcatgagaa
agttagaaccagaagaatttgcagcatatcttgaaccattcaaagagaaaggtgaagttcgtcgtccaacattatcatggcctcgtgaa
atcccgttagtaaaaggtggtaaacctgacgttgtacaaattgttaggaattataatgcttatctacgtgcaagtgatgatttaccaaaaat
gtttattgaatcggacccaggattcttttccaatgctattgttgaaggtgccaagaagtttcctaatactgaatttgtcaaagtaaaaggtctt
catttttcgcaagaagatgcacctgatgaaatgggaaaatatatcaaatcgttcgttgagcgagttctcaaaaatgaacaataaGTC
GAC (Sall)
DYR Protein Sequence (SEQ ID NO: 2):
MISLIAALAVDRVIGMENAMPWNLPADLAWFKRNTLNKPVIMGRHTWESIGRPLPGR
KNIILSSQPGTDDRVTWVKSVDEAIAACGDVPEIMVIGGGRVYEQFLPKAQKLYLTHIDAEVEGD
THFPDYEPDDWESVFSEFHDADAQNSHSYCFEILERRACVSKGEELFTGVVPILVELDGDVNG
HKFSVSGEGEGDATYGKLTLKFICTTGKLPVPWPTLVTTFGYGLQCFARYPDHMKQHDFFKSA
MPEGYVQERTIFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSYQSALSKDPNEKR
DHMVLLEFVTAAGITLGMDELYKGSGEGRGSLLTCGDVEENPGPASTSKVYDPEQRKRMITGP
QWWARCKQMNVLDSFINYYDSEKHAENAVIFLHGNAASSYLWRHVVPHIEPVARCIIPDLIGMG
KSGKSGNGSYRLLDHYKYLTAWFELLNLPKKIIFVGHDWGACLAFHYSYEHQDKIKAIVHAESV
VDVIESWDEWPDIEEDIALIKSEEGEKMVLENNFFVETMLPSKIMRKLEPEEFAAYLEPFKEKGE
VRRPTLSWPREIPLVKGGKPDVVQIVRNYNAYLRASDDLPKMFIESDPGFFSNAIVEGAKKFPN
TEFVKVKGLHFSQEDAPDEMGKYIKSFVERVLKNEQ
pELPS DYR Vector Sequence (SEQ ID NO: 3):
ATTGATCACGTGAGGCTCCGGTGCCCGTCAGTGGGCAGAGCGCACATCGCCCA
CAGTCCCCGAGAAGTTGGGGGGAGGGGTTTTATGCGATGGAGTTTCCCCACACTGAGTGG
GTGGAGACTGAAGTTAGGCCAGCTTGGCACTTGATGTAATTCTCCTTGGAATTTGCCCTTT
TTGAGTTTGGATCTTGGTTCATTCTCAAGCCTCAGACAGTGGTTCAAAGTTTTTTTCTTCCAT
TTCAGGTGTCGTGATCTAGAGGATCCATGATAAGTTTGATTGCTGCTCTGGCTGTGGACCG
GGTAATCGGTATGGAAAACGCCATGCCCTGGAACCTGCCTGCCGATTTGGCTTGGTTCAA
GCGCAATACCCTGAACAAACCAGTAATCATGGGAAGGCATACATGGGAAAGCATTGGAAG
ACCACTTCCCGGTAGAAAGAATATTATCCTGTCTAGCCAGCCCGGCACGGATGATAGGGT
GACATGGGTAAAGAGCGTCGATGAGGCGATTGCGGCGTGTGGTGACGTGCCGGAAATTAT
GGTTATCGGAGGCGGCAGGGTCTACGAACAGTTCCTGCCGAAGGCACAGAAGCTGTACCT
CACCCACATCGATGCAGAGGTGGAAGGAGACACGCACTTTCCAGATTACGAGCCTGATGA
CTGGGAGAGTGTTTTTAGCGAATTCCATGACGCAGACGCCCAAAACTCTCACTCCTACTGC
TTTGAGATTCTCGAACGAAGGgcatgcgtgagcaagggcgaggagctgttcaccggggtggtgcccatcctggtcga
gctggacggcgacgtaaacggccacaagttcagcgtgtccggcgagggcgagggcgatgccacctacggcaagctgaccctgaa
gttcatctgcaccaccggcaagctgcccgtgccctggcccaccctcgtgaccaccttcggctacggcctgcagtgcttcgcccgctacc
ccgaccacatgaagcagcacgacttcttcaagtccgccatgcccgaaggctacgtccaggagcgcaccatcttcttcaaggacgac
ggcaactacaagacccgcgccgaggtgaagttcgagggcgacaccctggtgaaccgcatcgagctgaagggcatcgacttcaag
gaggacggcaacatcctggggcacaagctggagtacaactacaacagccacaacgtctatatcatggccgacaagcagaagaa
cggcatcaaggtgaacttcaagatccgccacaacatcgaggacggcagcgtgcagctcgccgaccactaccagcagaacacccc
catcggcgacggccccgtgctgctgcccgacaaccactacctgagctaccagtccgccctgagcaaagaccccaacgagaagcg
cgatcacatggtcctgctggagttcgtgaccgccgccgggatcactctcggcatggacgagctgtacaagGGCAGCGGAGA
GGGCAGAGGAAGTOTTCTAACATGCGGTGACGTGGAGGAGAATCCCGGCCCTgctagcacttc
gaaagtttatgatccagaacaaaggaaacggatgataactggtccgcagtggtgggccagatgtaaacaaatgaatgttcttgattca
tttattaattattatgattcagaaaaacatgcagaaaatgctgttatttttttacatggtaacgcggcctcttcttatttatggcgacatgttgtgc
cacatattgagccagtagcgcggtgtattataccagaccttattggtatgggcaaatcaggcaaatctggtaatggttcttataggttactt
gatcattacaaatatcttactgcatggtttgaacttcttaatttaccaaagaagatcatttttgtcggccatgattggggtgcttgtttggcatttc
attatagctatgagcatcaagataagatcaaagcaatagttcacgctgaaagtgtagtagatgtgattgaatcatgggatgaatggcct
gatattgaagaagatattgcgttgatcaaatctgaagaaggagaaaaaatggttttggagaataacttcttcgtggaaaccatgttgcca
tcaaaaatcatgagaaagttagaaccagaagaatttgcagcatatcttgaaccattcaaagagaaaggtgaagttcgtcgtccaacat
tatcatggcctcgtgaaatcccgttagtaaaaggtggtaaacctgacgttgtacaaattgttaggaattataatgcttatctacgtgcaagt
gatgatttaccaaaaatgtttattgaatcggacccaggattcttttccaatgctattgttgaaggtgccaagaagtttcctaatactgaatttgt
caaagtaaaaggtcttcatttttcgcaagaagatgcacctgatgaaatgggaaaatatatcaaatcgttcgttgagcgagttctcaaaa
atgaacaataaGTCGACAATCAACCTCTGGATTACAAAATTTGTGAAAGATTGACTGGTATTCTT
AACTATGTTGCTCCTTTTACGCTATGTGGATACGCTGCTTTAATGCCTTTGTATCATGCTATT
GCTTCCCGTATGGCTTTCATTTTCTCCTCCTTGTATAAATCCTGGTTGCTGTCTCTTTATGA
GGAGTTGTGGCCCGTTGTCAGGCAACGTGGCGTGGTGTGCACTGTGTTTGCTGACGCAAC
CCCCACTGGTTGGGGCATTGCCACCACCTGTCAGCTCCTTTCCGGGACTTTCGCTTTCCC
CCTCCCTATTGCCACGGCGGAACTCATCGCCGCCTGCCTTGCCCGCTGCTGGACAGGGG
CTCGGCTGTTGGGCACTGACAATTCCGTGGTGTTGTCGGGGAAGCTGACGTCCTTTCCAT
GGCTGCTCGCCTGTGTTGCCACCTGGATTCTGCGCGGGACGTCCTTCTGCTACGTCCCTT
CGGCCCTCAATCCAGCGGACCTTCCTTCCCGCGGCCTGCTGCCGGCTCTGCGGCCTCTTC
CGCGTCTTCGCCTTCGCCCTCAGACGAGTCGGATCTCCCTTTGGGCCGCCTCCCCGCCTG
GAATTCGAGCTCGGTACCTTTAAGACCAATGACTTACAAGGCAGCTGTAGATCTTAGCCAC
TTTTTAAAAGAAAAGGGGGGACTGGAAGGGCTAATTCACTCCCAACGAAGACAAGATCTGC
TTTTTGCTTGTACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAGTAGTGTGT
GCCCGTCTGTTGTGTGACTCTGGTAACTAGAGATCCCTCAGACCCTTTTAGTCAGTGTGGA
AAATCTCTAGCAGTAGTAGTTCATGTCATCTTATTATTCAGTATTTATAACTTGCAAAGAAAT
GAATATCAGAGAGTGAGAGGAACTTGTTTATTGCAGCTTATAATGGTTACAAATAAAGCAAT
AGCATCACAAATTTCACAAATAAAGCATTTTTTTCACTGCATTCTAGTTGTGGTTTGTCCAAA
CTCATCAATGTATCTTATCATGTCTGGCTCTAGCTATCCCGCCCCTAACTCCGCCCATCCC
GCCCCTAACTCCGCCCAGTTCCGCCCATTCTCCGCCCCATGGCTGACTAATTTTTTTTATTT
ATGCAGAGGCCGAGGCCGCCTCGGCCTCTGAGCTATTCCAGAAGTAGTGAGGAGGCTTTT
TTGGAGGCCTAGGGACGTACCCAATTCGCCCTATAGTGAGTCGTATTACGCGCGCTCACT
GGCCGTCGTTTTACAACGTCGTGACTGGGAAAACCCTGGCGTTACCCAACTTAATCGCCTT
GCAGCACATCCCCCTTTCGCCAGCTGGCGTAATAGCGAAGAGGCCCGCACCGATCGCCCT
TCCCAACAGTTGCGCAGCCTGAATGGCGAATGGCGCGACGCGCCCTGTAGCGGCGCATT
AAGCGCGGCGGGTGTGGTGGTTACGCGCAGCGTGACCGCTACACTTGCCAGCGCCCTAG
CGCCCGCTCCTTTCGCTTTCTTCCCTTCCTTTCTCGCCACGTTCGCCGGCTTTCCCCGTCA
AGCTCTAAATCGGGGGCTCCCTTTAGGGTTCCGATTTAGTGCTTTACGGCACCTCGACCCC
AAAAAACTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGAACAAC
ACTCAACCCTATCTCGGTCTATTCTTTTGATTTATAAGGGATTTTGCCGATTTCGGCCTATT
GGTTAAAAAATGAGCTGATTTAACAAAAATTTAACGCGAATTTTAACAAAATATTAACGTTTA
CAATTTCCCAGGTGGCACTTTTCGGGGAAATGTGCGCGGAACCCCTATTTGTTTATTTTTCT
AAATACATTCAAATATGTATCCGCTCATGAGACAATAACCCTGATAAATGCTTCAATAATATT
GAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCA
GTTGGGTGCACGAGTGGGTTACATCGAACTGGATCTCAACAGCGGTAAGATCCTTGAGAG
TTTTCGCCCCGAAGAACGTTTTCCAATGATGAGCACTTTTAAAGTTCTGCTATGTGGCGCG
GTATTATCCCGTATTGACGCCGGGCAAGAGCAACTCGGTCGCCGCATACACTATTCTCAGA
ATGACTTGGTTGAGTACTCACCAGTCACAGAAAAGCATCTTACGGATGGCATGACAGTAAG
AGAATTATGCAGTGCTGCCATAACCATGAGTGATAACACTGCGGCCAACTTACTTCTGACA
ACGATCGGAGGACCGAAGGAGCTAACCGCTTTTTTGCACAACATGGGGGATCATGTAACT
CGCCTTGATCGTTGGGAACCGGAGCTGAATGAAGCCATACCAAACGACGAGCGTGACACC
ACGATGCCTGTAGCAATGGCAACAACGTTGCGCAAACTATTAACTGGCGAACTACTTACTC
TAGCTTCCCGGCAACAATTAATAGACTGGATGGAGGCGGATAAAGTTGCAGGACCACTTCT
GCGCTCGGCCCTTCCGGCTGGCTGGTTTATTGCTGATAAATCTGGAGCCGGTGAGCGTGG
GTCTCGCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGAGATAGG
TGCCTCACTGATTAAGCATTGGTAACTGTCAGACCAAGTTTACTCATATATACTTTAGATTG
ATTTAAAACTTCATTTTTAATTTAAAAGGATCTAGGTGAAGATCCTTTTTGATAATCTCATGA
CCAAAATCCCTTAACGTGAGTTTTCGTTCCACTGAGCGTCAGACCCCGTAGAAAAGATCAA
AGGATCTTCTTGAGATCCTTTTTTTCTGCGCGTAATCTGCTGCTTGCAAACAAAAAAACCAC
CGCTACCAGCGGTGGTTTGTTTGCCGGATCAAGAGCTACCAACTCTTTTTCCGAAGGTAAC
TGGCTTCAGCAGAGCGCAGATACCAAATACTGTCCTTCTAGTGTAGCCGTAGTTAGGCCAC
CACTTCAAGAACTCTGTAGCACCGCCTACATACCTCGCTCTGCTAATCCTGTTACCAGTGG
CTGCTGCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACGCTTCCC
GAAGGGAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGAAAAACG
CCAGCAACGCGGCCTTTTTACGGTTCCTGGCCTTTTGCTGGCCTTTTGCTCACATGTTCTTT
CCTGCGTTATCCCCTGATTCTGTGGATAACCGTATTACCGCCTTTGAGTGAGCTGATACCG
CTCGCCGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATGCAGCTGGCACGA
CAGGTTTCCCGACTGGAAAGCGGGCAGTGAGCGCAACGCAATTAATGTGAGTTAGCTCAC
TCATTAGGCACCCCAGGCTTTACACTTTATGCTTCCGGCTCGTATGTTGTGTGGAATTGTG
AGCGGATAACAATTTCACACAGGAAACAGCTATGACCATGATTACGCCAAGCGCGCAATTA
ACCCTCACTAAAGGGAACAAAAGCTGGAGCTGCAAGCTTAATGTAGTCTTATGCAATACTC
TTGTAGTCTTGCAACATGGTAACGATGAGTTAGCAACATGCCTTACAAGGAGAGAAAAAGC
ACCGTGCATGCCGATTGGTGGAAGTAAGGTGGTACGATCGTGCCTTATTAGGAAGGCAAC
AGACGGGTCTGACATGGATTGGACGAACCACTGAATTGCCGCATTGCAGAGATATTGTATT
TAAGTGCCTAGCTCGATACATTAAACGGGTCTCTCTGGTTAGACCAGATCTGAGCCTGGGA
GCTCTCTGGCTAACTAGGGAACCCACTGCTTAAGCCTCAATAAAGCTTGCCTTGAGTGCTT
CAAGTAGTGTGTGCCCGTCTGTTGTGTGACTCTGGTAACTAGAGATCCCTCAGACCCTTTT
AGTCAGTGTGGAAAATCTCTAGCAGTGGCGCCCGAACAGGGACCTGAAAGCGAAAGGGAA
ACCAGAGCTCTCTCGACGCAGGACTCGGCTTGCTGAAGCGCGCACGGCAAGAGGCGAGG
GGCGGCGACTGGTGAGTACGCCAAAAATTTTGACTAGCGGAGGCTAGAAGGAGAGAGATG
GGTGCGAGAGCGTCAGTATTAAGCGGGGGAGAATTAGATCGCGATGGGAAAAAATTCGGT
TAAGGCCAGGGGGAAAGAAAAAATATAAATTAAAACATATAGTATGGGCAAGCAGGGAGCT
AGAACGATTCGCAGTTAATCCTGGCCTGTTAGAAACATCAGAAGGCTGTAGACAAATACTG
GGACAGCTACAACCATCCCTTCAGACAGGATCAGAAGAACTTAGATCATTATATAATACAGT
AGCAACCCTCTATTGTGTGCATCAAAGGATAGAGATAAAAGACACCAAGGAAGCTTTAGAC
AAGATAGAGGAAGAGCAAAACAAAAGTAAGACCACCGCACAGCAAGCGGCCGCTGATCTT
CAGACCTGGAGGAGGAGATATGAGGGACAATTGGAGAAGTGAATTATATAAATATAAAGTA
GTAAAAATTGAACCATTAGGAGTAGCACCCACCAAGGCAAAGAGAAGAGTGGTGCAGAGA
GAAAAAAGAGCAGTGGGAATAGGAGCTTTGTTCCTTGGGTTCTTGGGAGCAGCAGGAAGC
ACTATGGGCGCAGCGTCAATGACGCTGACGGTACAGGCCAGACAATTATTGTCTGGTATA
GTGCAGCAGCAGAACAATTTGCTGAGGGCTATTGAGGCGCAACAGCATCTGTTGCAACTC
ACAGTCTGGGGCATCAAGCAGCTCCAGGCAAGAATCCTGGCTGTGGAAAGATACCTAAAG
GATCAACAGCTCCTGGGGATTTGGGGTTGCTCTGGAAAACTCATTTGCACCACTGCTGTGC
CTTGGAATGCTAGTTGGAGTAATAAATCTCTGGAACAGATTTGGAATCACACGACCTGGAT
GGAGTGGGACAGAGAAATTAACAATTACACAAGCTTAATACACTCCTTAATTGAAGAATCGC
AAAACCAGCAAGAAAAGAATGAACAAGAATTATTGGAATTAGATAAATGGGCAAGTTTGTG
GAATTGGTTTAACATAACAAATTGGCTGTGGTATATAAAATTATTCATAATGATAGTAGGAG
GCTTGGTAGGTTTAAGAATAGTTTTTGCTGTACTTTCTATAGTGAATAGAGTTAGGCAGGGA
TATTCACCATTATCGTTTCAGACCCACCTCCCAACCCCGAGGGGACCCGACAGGCCCGAA
GGAATAGAAGAAGAAGGTGGAGAGAGAGACAGAGACAGATCCATTCGATTAGTGAACGGA
TCTCGACGGTATCGATTAGACTGTAGCCCAGGAATATGGCAGCTAGATTGTACACATTTAG
AAGGAAAAGTTATCTTGGTAGCAGTTCATGTAGCCAGTGGATATATAGAAGCAGAAGTAATT
CCAGCAGAGACAGGGCAAGAAACAGCATACTTCCTCTTAAAATTAGCAGGAAGATGGCCA
GTAAAAACAGTACATACAGACAATGGCAGCAATTTCACCAGTACTACAGTTAAGGCCGCCT
GTTGGTGGGCGGGGATCAAGCAGGAATTTGGCATTCCCTACAATCCCCAAAGTCAAGGAG
TAATAGAATCTATGAATAAAGAATTAAAGAAAATTATAGGACAGGTAAGAGATCAGGCTGAA
CATCTTAAGACAGCAGTACAAATGGCAGTATTCATCCACAATTTTAAAAGAAAAGGGGGGA
TTGGGGGGTACAGTGCAGGGGAAAGAATAGTAGACATAATAGCAACAGACATACAAACTAA
AGAATTACAAAAACAAATTACAAAAATTCAAAATTTTCGGGTTTATTACAGGGACAGCAGAG
ATCCAGTTTGGCTGC

### Example 1: Synthesis of [¹¹C]TMP

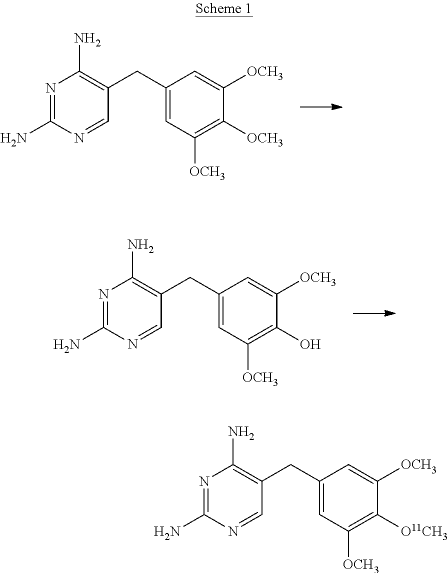

[¹¹C]TMP was prepared according to the procedure in ChemBioChem 2007, 8, 767-774. See, Scheme 1. Specifically, Trimethoprim (3 g, 10.03 mmol, purchased from Sigma-Aldrich) is selectively demethylated by HBr (37.4 mL, 48% in water) for 20 min at 95° C. After the reaction, the reaction mixture was cooled down and NaOH (8.92 mL, 50% w/w) added. After precipitation, the precipitate was filtered and collected, and re-dissolved in boiling water. NH₄OH was added to the mixture solution until pH 7, recrystallized at 4° C., filtered, and collected. 5-(3,5-Dimethoxy-4-hydroxy-benzyl)pyrimidine-2,4-diamine was obtained as a pink solid (1.51 g) in 52.9% yield; ¹H NMR (DMSO-d₆) δ 8.06 (s, —OH), 7.45 (s, 1H), 6.48 (s, 2H), 5.99 (s, —NH₂), 5.63 (s, —NH₂), 3.71 (s, 6H), 3.47 (s, 2H).

5-(3,5-Dimethoxy-4-hydroxy-benzyl)pyrimidine-2,4-diamine was reacted with ¹¹CH₃I for 5 min at 70° C. in the present of 5 N NaOH (4 μL) as a base. After the reaction, the reaction mixture was purified by HPLC with 12% EtOH in 0.01 M Phosphate buffer (pH=3). The flow rate of HPLC was 3 mL/min and the product ([¹¹C]TMP) was eluted at 12 min retention time. The radiochemical yield was 40-50% from [¹¹C]CH₃I, radiochemical purity was over 99%, and the specific activity was 37-56 GBq/μmol.

### Example 2: Synthesis of Cold Fluoropropyl-TMP (FP-TMP)

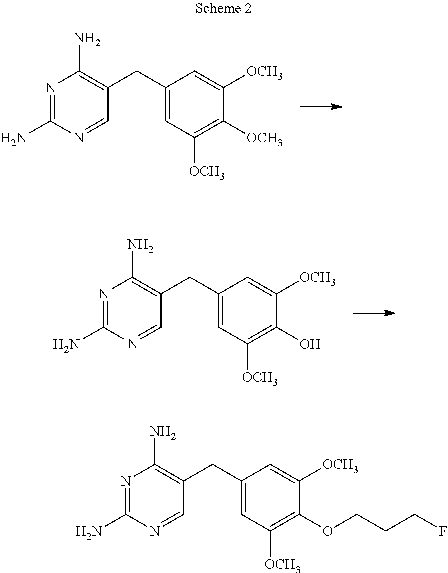 [SMILES: COc1cc(Cc2cnc(N)nc2N)cc(OC)c1OC ; COc1cc(Cc2cnc(N)nc2N)cc(OC)c1O ; COc1cc(Cc2cnc(N)nc2N)cc(OC)c1OCCCF]

FP-TMP was prepared according to the transformations of Scheme 2. Specifically, trimethoprim (3 g, 10.3 mmol) was reacted with HBr (37.4 mL, 48% in water) as described in Example 1.

FP-TMP was prepared from 5-(3,5-dimethoxy-4-hydroxy-benzyl)pyrimidine-2,4-diamine (500 mg, 1.81 mmol) with 1-bromopropyl-3-fluoride (510.4 mg, 3.62 mmol) and cesium carbonate (1.18 g, 3.62 mmol) in DMF (25.0 mL). The reaction mixture was stirred at 80° C. for 4 hours. After the reaction, the solvent was removed in vacuo, flash column (CH₂Cl₂:MeOH=15:1) gave FP-TMP as a yellow solid (166.9 mg) in a 27.4% yield; ¹H NMR (DMSO-d₆) δ 7.51 (s, 1H), 6.54 (s, 2H), 6.05 (s, —NH₂), 5.66 (s, —NH₂), 4.62 (dt, J=46.8 and 7.2 Hz, 2H), 3.89 (t, J=7.2 Hz, 2H), 3.71 (s, 6H), 3.52 (s, 2H), 1.94 (dq, J=25.2 and 7.2 Hz, 2H).

### Example 3: Synthesis of [¹⁸F]FP-TMP

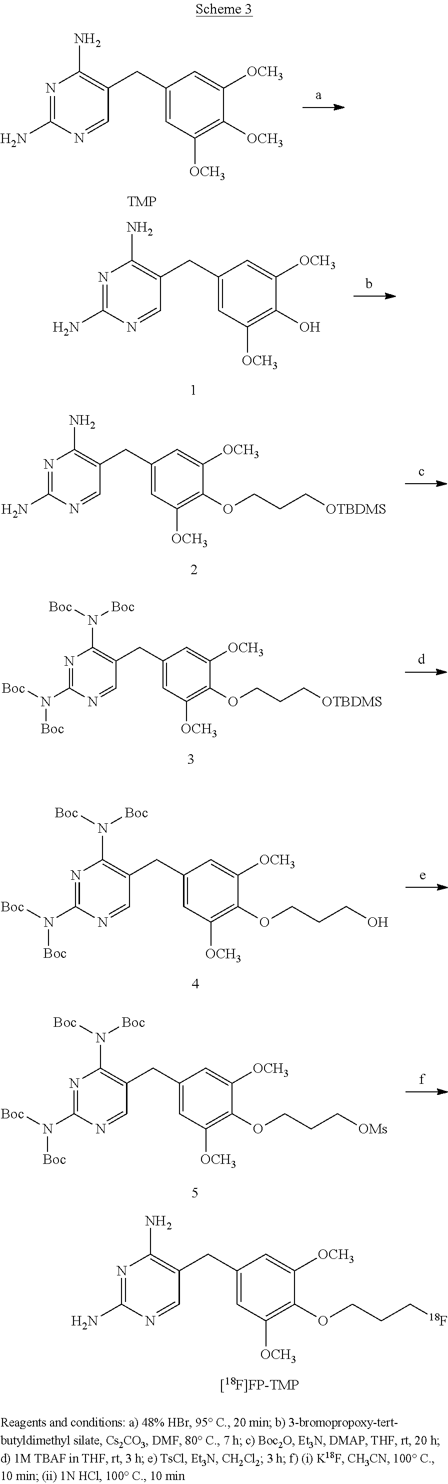

[¹⁸F]Fluoroethyl-TMP was prepared according to the transformations of Scheme 3. Specifically, trimethoprim (3 g, 10.3 mmol) was reacted with HBr (37.4 mL, 48% in water) as described in Example 1.

The hydroxyl moiety of 4-((2,4-diaminopyrimidin-5-yl)methyl)-2,6-dimethoxyphenol (1, 43 mg, 0.15 mmol) was substituted using 3-bromopropoxy-tert-butyldimethyl silate (78.8 mg, 0.31 mmol), cesium carbonate (101.3 mg, 0.31 mmol) in DMF (2.14 mL) at 80° C. for 7 h. DMF was removed in vacuo, flash column gave 5-(4-(3-((tert-butyldimethylsilyl)oxy)propoxy)-3,5-dimethoxybenzyl)pyrimidine-2,4-diamine (2) as a light yellow solid (20 mg) in a 28.7% yield; ¹H NMR (CDCl₃) δ 7.78 (s, 2H), 6.38 (s, 2H), 4.73 (—NH₂, 2H), 4.55 (—NH₂, 2H), 4.05 (t, J=7.2 Hz, 2H), 3.83-3.79 (m, 2H), 3.78 (s, 6H), 3.65 (s, 2H), 1.94 (t, J=7.2 Hz, 2H), 0.88 (s, 9H), 0.06 (s, 6H).

The amine groups of 5-(4-(3-((tert-butyldimethylsilyl)oxy)propoxy)-3,5-dimethoxybenzyl)pyrimidine-2,4-diamine (400 mg, 0.89 mmol) were then bis-BOC protected using Boc₂O (655.9 μL, 2.67 mmol), triethylamine (496.6 μL, 2.67 mmol), dimethylaminopyridine (75.6 mg, 0.26 mmol) in tetrahydrofuran (12 mL) at room temperature for 20 hours. The reaction mixture was diluted with water (50 mL), extracted with CH₂Cl₂ (50 mL) twice, washed with brine (50 mL), and then dried over with anhydrous sodium sulfate, concentrated. Flash column gave di-tert-butyl (5-(4-(3-((tert-butyldimethylsilyl)oxy)propoxy)-3,5-dimethoxybenzyl)pyrimidine-2,4-diyl)bis((tert-butoxycarbonyl)carbamate) (3) as a white solid (195.9 mg) in a 25.8% yield; ¹H NMR (CDCl₃) δ 8.56 (s, 1H), 6.37 (s, 2H), 4.04 (t, J=7.2 Hz, 2H), 3.83-3.82 (m, 4H), 3.78 (s, 6H), 1.95 (q, J=7.2 Hz, 2H), 1.45 (s, 18H), 1.39 (s, 18H), 0.89 (s, 9H), 0.58 (s, 6H).

The silate moiety of di-tert-butyl (5-(4-(3-((tert-butyldimethylsilyl)oxy)propoxy)-3,5-dimethoxybenzyl)pyrimidine-2,4-diyl)bis((tert-butoxycarbonyl)carbamate) (3) (195.5 mg, 0.23 mmol) was then removed using 1 M tetrabutylammonium fluoride in tetrahydrofuran (0.69 mL, 0.69 mmol) and tetrahydrofuran (6.9 mL) at room temperature for 2 hours. The reaction mixture was diluted with water (50 mL), extracted with ethyl acetate (50 mL) twice, washed with brine (50 mL), and then dried over with anhydrous sodium sulfate, concentrated. Flash column (hexane:ethyl acetate=2:3) gave di-tert-butyl (5-(4-(3-hydroxypropoxy)-3,5-dimethoxybenzyl)pyrimidine-2,4-diyl)bis((tert-butoxycarbonyl)carbamate) (4) as a colorless oil (146.6 mg) in an 86.7% yield; ¹H NMR (CDCl₃) δ 8.56 (s, 1H), 6.39 (s, 2H), 4.13 (t, J=7.2 Hz, 2H), 3.90 (t, J=7.2 Hz, 2H), 3.82 (s, 2H), 3.81 (s, 6H), 3.68 (t, J=3.6 Hz, 2H), 1.96 (q, J=7.2 Hz, 2H), 1.54 (s, 18H), 1.45 (s, 9H), 1.39 (s, 9H).

The propoxy moiety of di-tert-butyl (5-(4-(3-hydroxypropoxy)-3,5-dimethoxybenzyl)pyrimidine-2,4-diyl)bis((tert-butoxycarbonyl)carbamate) (4) (137.6 mg, 0.18 mmol) was mesylated using mesyl chloride (43.5 μL, 0.56 mmol), triethyl amine (78.3 μL, 0.56 mmol) in CH₂Cl₂ (4 mL) for 2 hours at room temperature. The reaction mixture was diluted with water (20 mL), extracted with CH₂Cl₂ (20 mL) twice, washed with brine (20 mL), and then dried over with anhydrous sodium sulfate, concentrated. Flash column (hexane:ethyl acetate=1:2) gave 3-(4-((2,4-bis(bis(tert-butoxycarbonyl)amino)pyrimidin-5-yl)methyl)-2,6-dimethoxyphenoxy)propyl methanesulfonate (5) as a white solid (133.5 mg) in an 87.8% yield; ¹H NMR (CDCl₃) δ 8.55 (s, 1H), 6.38 (s, 2H), 4.54 (t, J=7.2 Hz, 2H), 4.05 (t, J=3.6 Hz, 2H), 3.81 (s, 2H), 3.79 (s, 6H), 3.03 (s, 3H), 1.45 (s, 18H), 1.39 (s, 18H).

Finally, the mesylate group of 3-(4-((2,4-bis(bis(tert-butoxycarbonyl)amino)pyrimidin-5-yl)methyl)-2,6-dimethoxyphenoxy)propyl methanesulfonate (5) (2 mg) was replaced with the ¹⁸F moiety using ¹⁸F-potassium fluoride and acetonitrile (200 μL) at 95° C. for 10 minutes. After the first reaction, 1 N HCl (1 mL) was added to a reaction mixture, and heated at 100° C. for 5 minutes. Followed by adding the HPLC eluent (1 mL), purified by HPLC (0.1% TFA in water:0.1% TFA in CH₃CN=80:20, flow rate=5 mL/min). The product ([¹⁸F]FP-TMP) was eluted from 23 to 25 min.

### Example 4: Synthesis of Halogen Radiolabeled Compounds

Compounds where R is a radioactive halogen may be prepared by replacing the “X” moiety in the following precursor compound which the appropriate halogen using reagents and skill in the art.

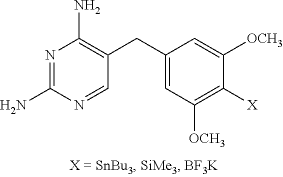

Similarly, the ²¹¹-At compounds may be prepared using the above-noted precursor compound where X is SnBu₃.

### Example 5: Synthesis of Glycol Compounds

Compounds where R is a glycol may be prepared by replacing the “X” moiety in the following precursor compounds which the appropriate glycol moiety using reagents and skill in the art.

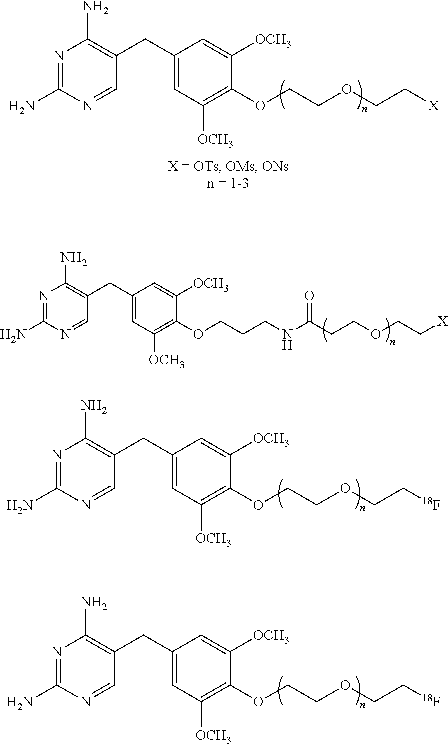

### Example 6: Synthesis of ¹⁸F-Phenyl Compounds

Compounds where R is a ¹⁸F-phenyl group may be prepared by replacing the “X” moiety in the following precursor compound with the appropriate phenyl moiety using reagents and skill in the art.

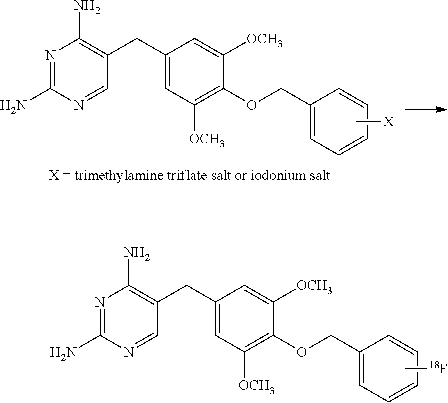

### Example 7: Synthesis of ¹⁸F-Triazole or Tetrazole Compounds

Compounds where R is a ¹⁸F-phenyl group may be prepared by replacing the “X” moiety in the following precursor compound with the appropriate triazole or tetrazole moiety using reagents and skill in the art.

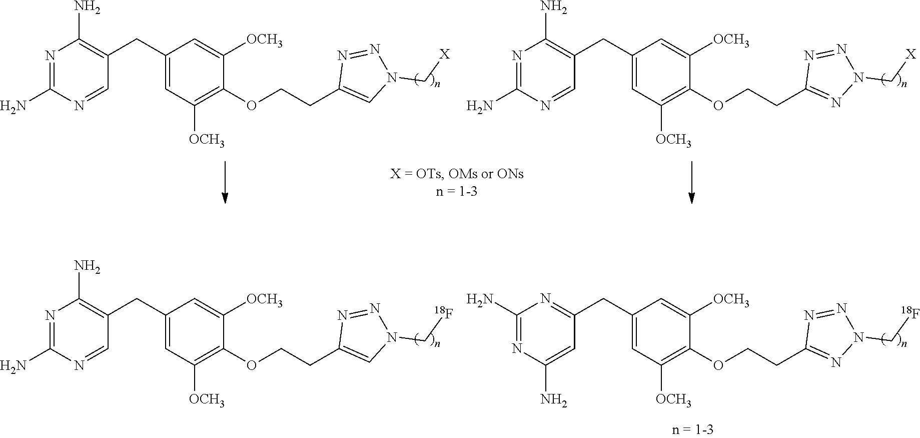

### Example 8: Synthesis of a ¹⁸F-BODIPY Compound

The following ¹⁸F-BODIPY compounds may be prepared by replacing the OTf moiety in the following precursor compound with ¹⁸F using reagents and skill in the art.

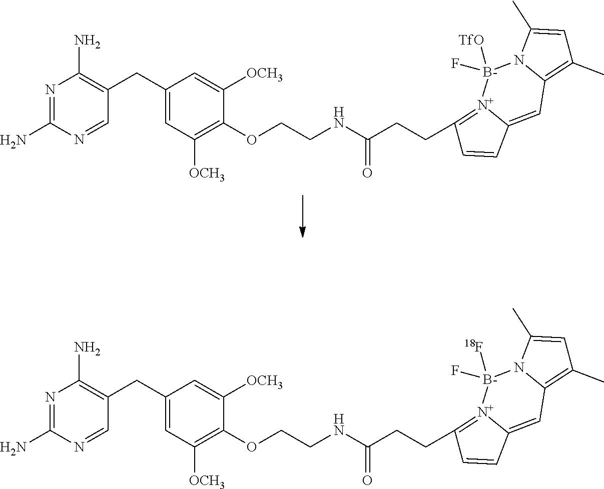 [SMILES: COc1cc(Cc2cnc(N)nc2N)cc(OC)c1OCCNC(=O)CCC1=[N+]2C(=Cc3c(C)cc(C)n3[B-]2(F)OS(=O)(=O)C(F)(F)F)C=C1 ; COc1cc(Cc2cnc(N)nc2N)cc(OC)c1OCCNC(=O)CCC1=[N+]2C(=Cc3c(C)cc(C)n3[B-]2(F)[18F])C=C1]

Accordingly, the following compounds having BODIPY structurally similar moieties may be prepared:

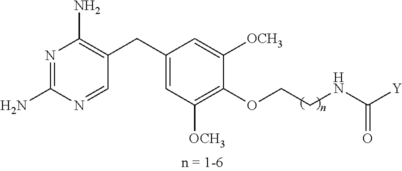

wherein Y is

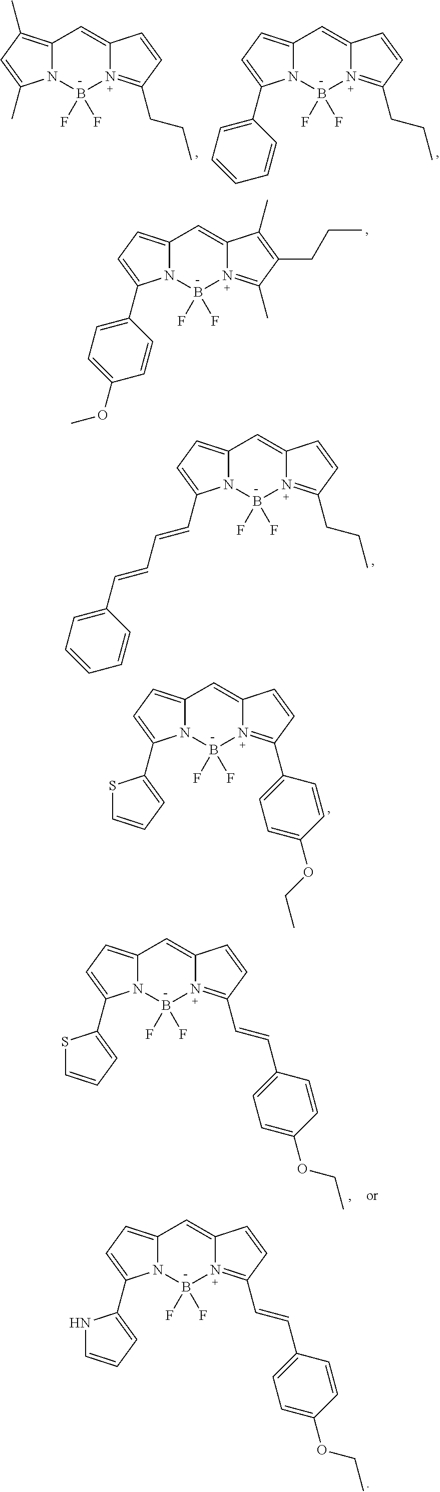

### Example 9: [¹¹C]TMP Binding

To assess [¹¹C]TMP binding to recombinant Ec DHFR protein (addgene, plasmid #29326) expressed in E coli (Invitrogen) and purified as detailed in Iwamoto, Chem. Biol., 17:981-988, and a dot blot assay was performed. Two L of Ec DHFR protein (as above) at varying concentrations of 100-4 M were dotted onto a nitrocellulose membrane (Abcam) dried for 1 h, blocked, and then incubated in 5% milk in TBS-T (20 mM Tris HCl, 150 mM NaCl, pH 7.5, 0.05% Tween 20), and incubated with [¹¹C]TMP (2 million CPM) for 30 minutes with or without cold TMP (10 μM) for 30 minutes. The blot was then washed with TBS×2 and exposed to a phosphor plate (GE) and imaged on a Typhoon laser scanner (GE).

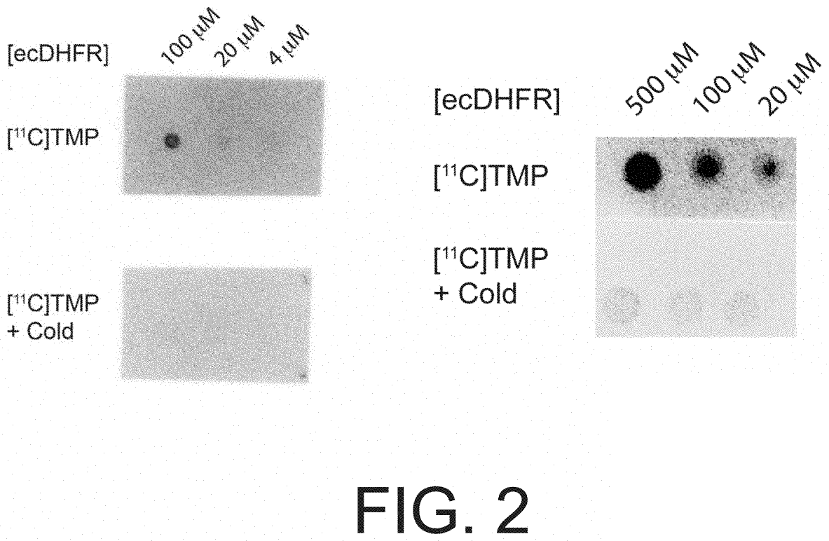

The dot blot of FIG. 2 shows [¹¹C]TMP concentration-dependent specific cell uptake using an in vitro dot blot assay with recombinant protein. Separately, robust uptake in cells carrying YFP-Ec DHFR fusions was seen.

### Example 10: Transgenic Cell Lines

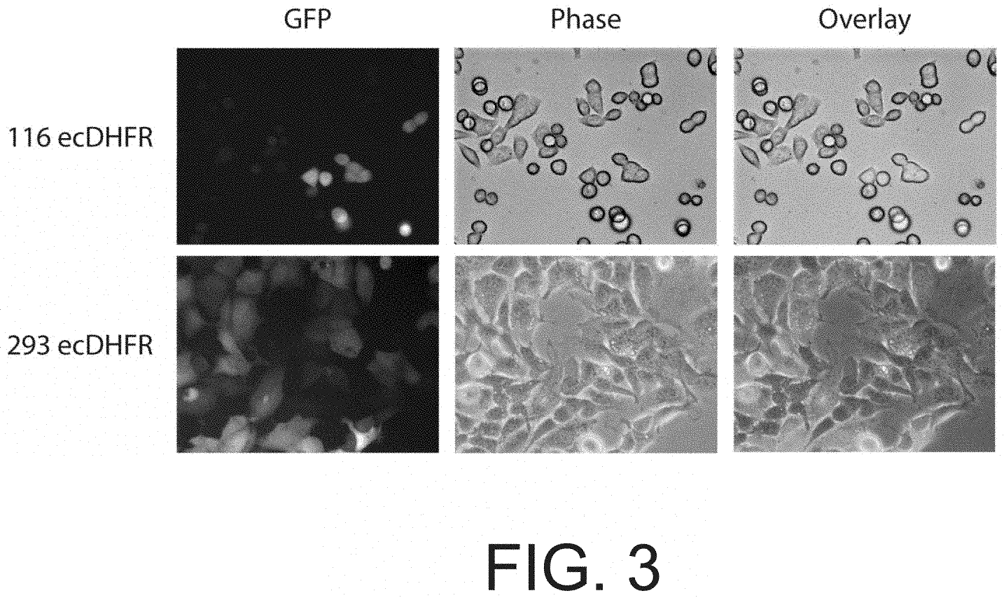

Transgenic mammalian cell lines carrying Ec DHFR were made using the Phoenix Amphotropic retroviral transduction. Specifically, these cell lines were prepared by retroviral transduction by transfecting Phoenix Amphotrophic cell lines (ATCC, # CRL-3213) with plasmid vector pBMN YFP-Ec-DHFR ires HcRed and used for YFP-Ec-DHFR expression using FACS (BD). Bright field and fluorescent microscopy was utilized to analyze the same and confirmed presence of the transgene in human embryonic kidney, HEK 293 cells (ATCC, # CCL-1573) and human colon carcinoma HCT 116 cells (ATCC, # CCL-247). See, FIG. 3.

The cells were lysed in radioimmunoprecipitation assay buffer (50 mM Tris, 150 mM sodium chloride, 1.0 mM EDTA, 1% Nonidet P40, and 0.25% SDS (pH 7.0)), supplemented with complete protease inhibitor cocktail (Roche) and phosphatase inhibitor cocktail 1 (Sigma Chemical Co). The cells were sonicated briefly, centrifuged at 13,000×g for 20 min at 4° C., and the supernatant collected. The protein concentration was determined using a Bio-Rad Dc protein assay kit (Bio-Rad Laboratories). Lysates containing 30 μg of protein were run on a 4-20% acrylamide gel and transferred to a PVDF membrane using the Trans-Blot Turbo Transfer System (Bio-Rad Laboratories). The PVDF membrane was incubated with Odyssey blocking buffer (Licor Biotechnology) for 1 h at room temperature, then overnight with a mouse monoclonal antibody recognizing YFP (catalogue #632381, Clontech) at a 1:1000 dilution at 4° C., and finally with the secondary antibody, IRDye 680RD goat anti-mouse IgG (Licor Biotechnology) at a 1:15,000 dilution. The same blot was incubated overnight with goat anti-GAPDH antibody (Santa Cruz) at a 1:300 dilution at 4° C. and then with IRDye 680RD donkey anti-goat IgG secondary antibody (Licor Biotechnology) at a 1:15,000 dilution. The signals were detected and quantified using the Odyssey® CLx Infrared Imaging System (Licor Biotechnology).

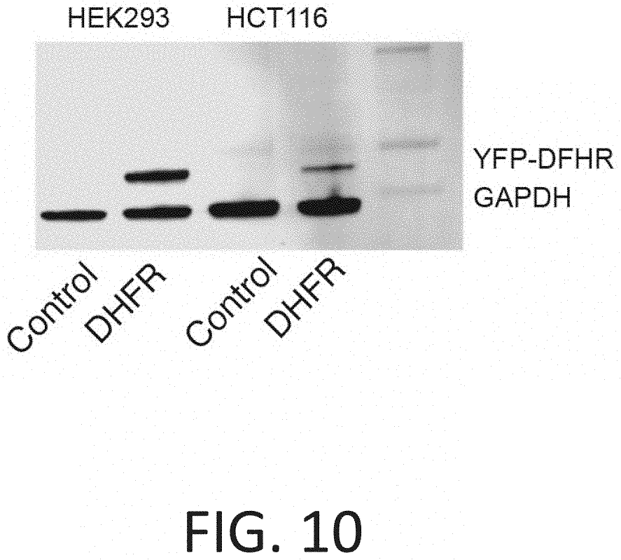

Western blotting confirmed the correct molecular weight (FIG. 10, 45 kDa).

### Example 11: [¹¹C]TMP HEK 293 Cell Uptake Studies

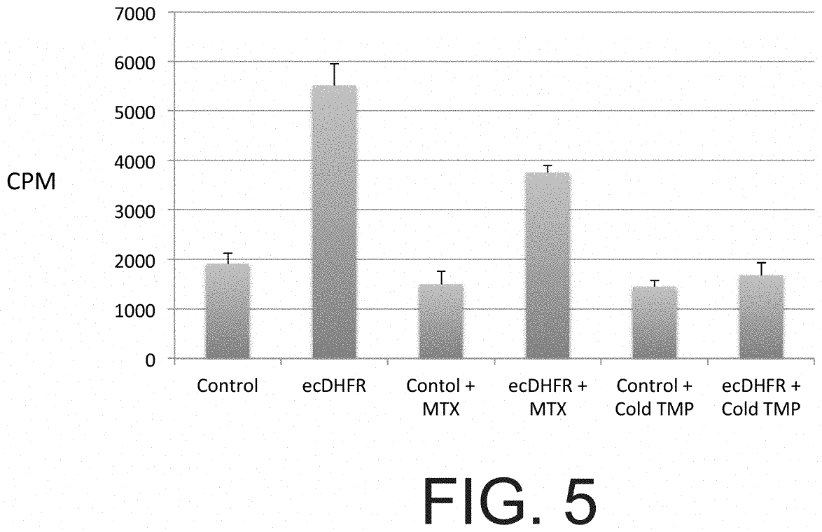

In this example, a HEK 293 cell uptake study was performed using TMP radiotracer cell uptake studies. Confluent HEK 293 control (ATCC, # CCL-1573; 8 million) or DHFR cells (high uptake) were trypsinized, incubated with [¹¹C]TMP with and without competing 10 μM cold TMP to block radiotracer uptake. Cells were washed twice with cold PBS and then counted with a gamma counter (FIG. 5).

These results show that there was over 10-fold signal induction at the time of assay with HEK293 cells and 3-fold induction with HCT116 cells, which correlated with differences in expression in the western blot.

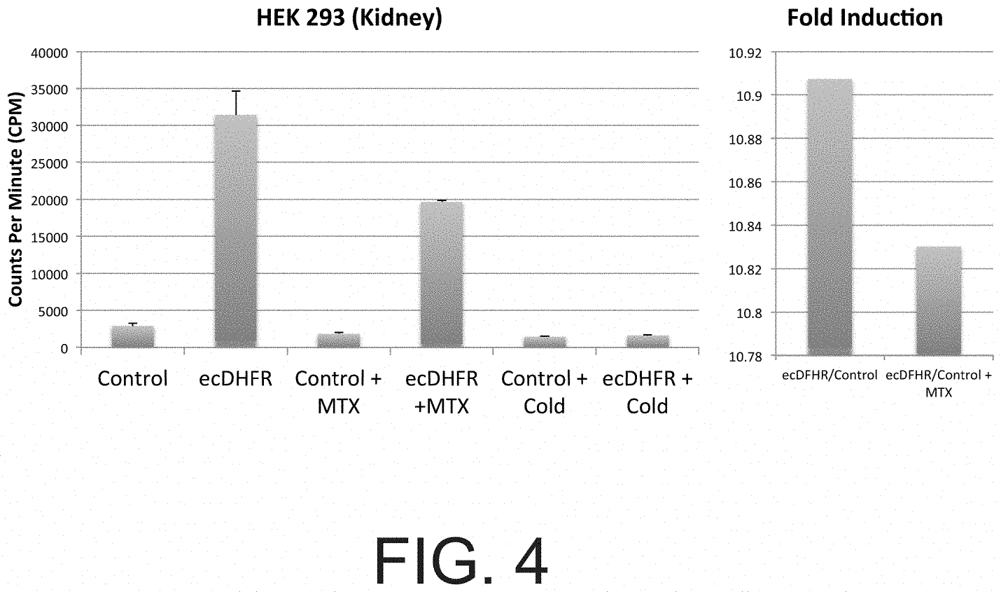

Given that 5 half-lives had occurred at the time of assay, less than 10% of the tracer bound to Ec DFHR remained detectable. This large percentage of decayed TMP competition suggests that TMP radiotracers labeled with longer-lived isotopes may demonstrate over 2 orders of magnitude of specific signal. Methotrexate co-treatment during uptake experiments did not change dynamic range of uptake and only marginally affected the absolute uptake numbers in HEK293 cells (FIG. 4).

### Example 12: [¹¹C]TMP Bacterial Cell Uptake Studies

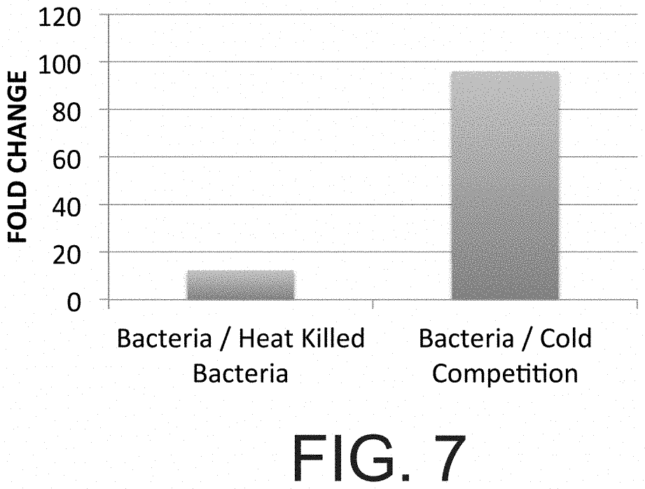

This example was performed to illustrate that TMP radiotracer derivatives may be used to specifically image bacteria. To this end, radiotracer bacterial cell uptake studies were completed using E. coli (Invitrogen; HB101). E. coli were grown overnight to saturation in standard LB media and then diluted 1:5 in fresh media 1 hour before incubation with radiotracer to ensure log phase growth. The experimental groups incubated with [¹¹C]TMP were Heat-killed Bacteria (low uptake), Bacteria (high uptake), and Bacteria+Cold (low uptake) indicating incubation with 10 M of cold TMP to block radiotracer uptake (FIGS. 6 and 7).

These bacterial uptake studies show over 10-fold specific uptake of [¹¹C]TMP in live bacteria compared with heat killed bacteria and that the uptake can be completely blocked with cold compound.

### Example 13: [¹¹C]TMP In Vivo Studies

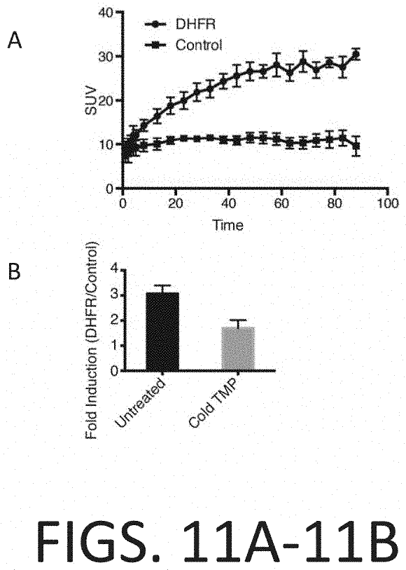

After successful in vitro uptake experiments, HCT116 tumor cells were xenografted into the posterior back/shoulder subcutaneous tissues of nude mice to assess in vivo tracer distribution and uptake. Tumors were grown over 10 days and animals were maintained on a low folate diet. Mice were anesthetized and imaged with small animal PET/CT. A representative image is shown at 90 minutes (5 min bin) after [¹¹C]TMP injection which shows strong signal coming from the DHFR carrying tumor cells. A time activity curve shows rapid and dynamic identification of DHFR carrying cells and over 3-fold signal induction with HCT116 cells compared to control cells, which correlated with in vitro uptake data (FIG. 11A). Other tissues with rapid uptake included the kidneys and bladder. There is decreased fold-induction (DHFR to control tumor signal) from the DHFR tumors after pre-treatment with cold oral TMP competition (0.2 mg/mL via the drinking water, FIG. 11B).

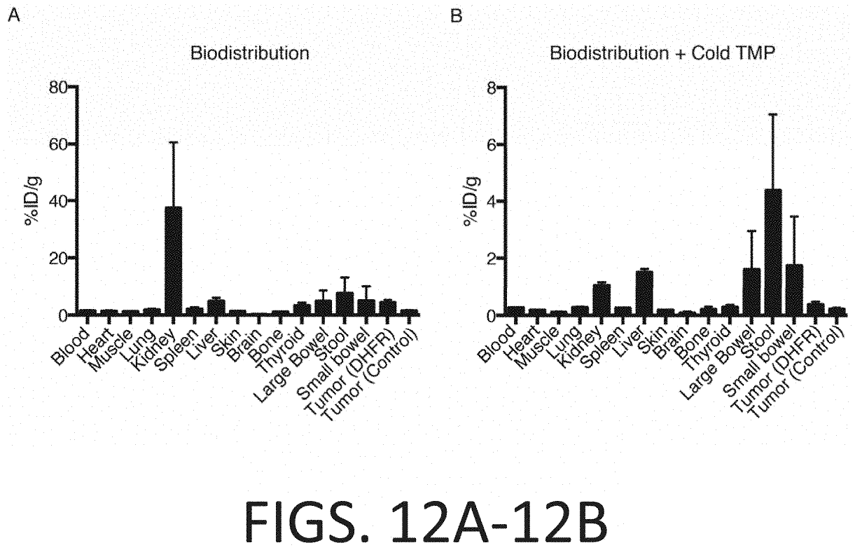

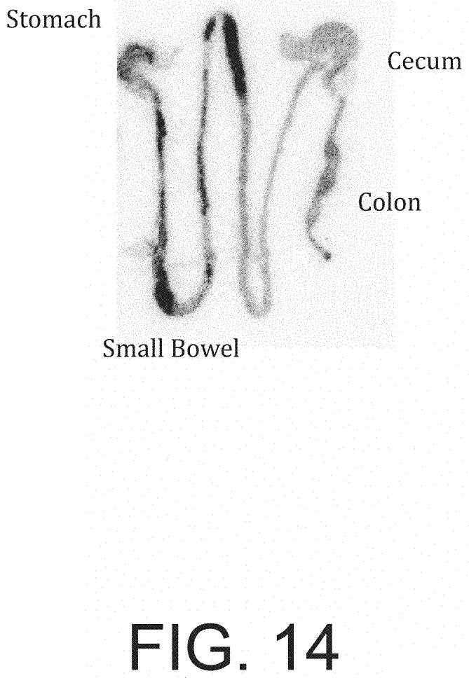

Biodistribution analysis was performed at 90 minutes after injection. Here again, DHFR to control signal induction showed over 3-fold signal induction in DHFR tumors versus control (FIG. 12A). There was significant uptake from the kidneys, but also modest liver uptake. Nonuniform increased signal in the small bowel after GI tract explant supports hepatobiliary excretion of the radiotracer (FIG. 14).

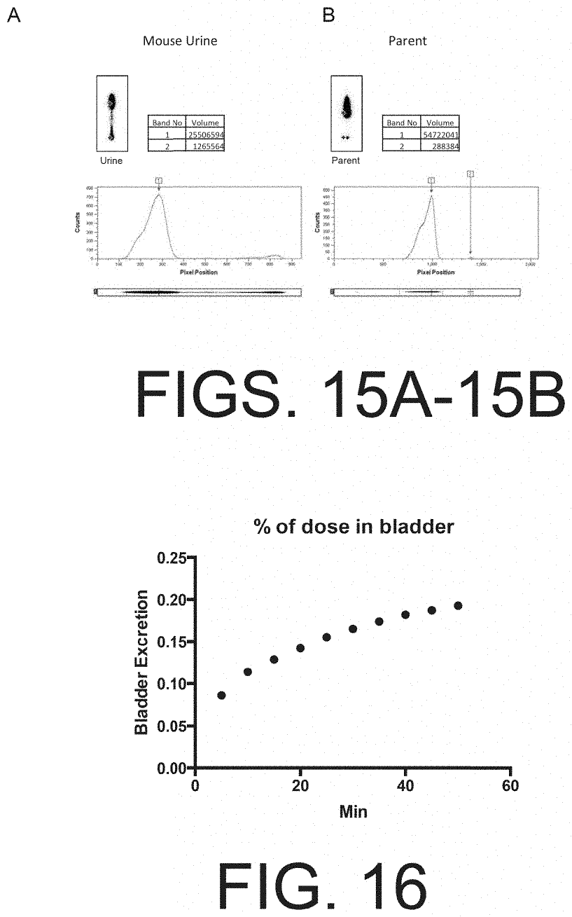

Notably, the signal in the small bowel appeared more concentrated than in the cecum/colon, the site of highest commensal bacterial colonization.

[¹¹C]TMP is excreted in the urine predominantly as the parent compound as measured by radio-thin layer chromatography (FIG. 15A/B) and there is rapid accumulation in the bladder (FIG. 16). The rate of urine filtration is rapid with approximately 20% of the dose rapidly accumulating in the bladder. Some of this prompt excretion may be related to the fact that mice have 8-10-fold circulating folate levels compared to humans. This excess of competing substrate may mildly affect DHFR active site occupancy, which could affect the overall signal to noise, thus mice are maintained on low folate, antibiotic free diets for 2 weeks prior to experimentation.

Additionally, there was biodistribution signal from the brain corroborating earlier studies that used TMP as a ligand to cross the blood brain barrier in rodents. In mice that had been pretreated with oral TMP there was a marked decreased in uptake in the kidneys without significant changes in other tissues (FIG. 12B).

In summary, the results with [¹¹C]TMP illustrate that there is little uptake in control cells in vitro. Additionally, co-treatment with cold TMP blocked specific uptake and co-treatment with inhibitory concentration of MTX showed no change in the dynamic range of uptake, and only minimal change in overall uptake in DHFR cells.

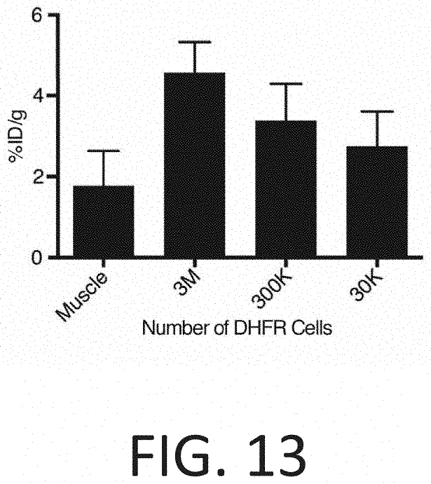

The number of cells detectable by imaging in a particular tissue/volume is crucial information, especially for investigators interested whether adoptive cell therapies are reaching the target solid tumor. Thus, cell sensitivity experiments were performed to assess the minimum number of cells necessary for detection of radiotracer signal. Given the strong in vitro signal uptake, HEK293 cells carrying DHFR transgene were diluted (3M, 300K, and 30K) and injected in 150 μL of matrigel matrix and imaged the next day. A representative mouse image is shown of the 300K cell shoulder area (FIG. 13), and ex vivo analysis by gamma counting corroborated detection of 300K cells (P<0.05).

### Example 14: [¹¹C]TMP Bacteria Comparison Studies

This example was performed to show the uptake of [¹¹C]TMP, initial dose of 2 million counts per minute CPM/mL on live bacteria over heat killed bacteria after a 30 minute incubation. The bacteria were then pelleted and washed in PBS twice before uptake was assessed on a gamma counter (Perkin Elmer). Several bacterial stains were tested including S. aureus (Xen 29, Perkin Elmer #119240) Pseudomonas (Xen 05 Perkin Elmer #119228) and E. coli (Xen 14 Perkin Elmer 119223).

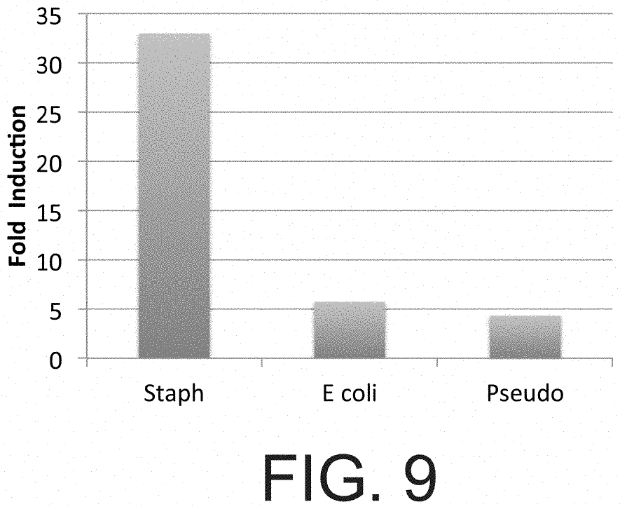

The results (FIG. 9) illustrate that a high fold-induction in all strains of bacteria tested and over 40 fold induction in Staphylococcus.

### Example 15: [¹¹C]TMP Bacteria Concentrations Study

This example was performed to study the effect of [¹¹C]TMP in the presence of varying E. coli (Invitrogen; HB101) bacterial concentrations from <1×10⁵ to 1×10⁷ colony forming units per mL. The [¹¹C]TMP initial dose was 2 million counts per minute (cpm)/mL, the bacteria were pelleted, washed twice in PBS and uptake was assessed with a gamma counter (Perkin Elmer).

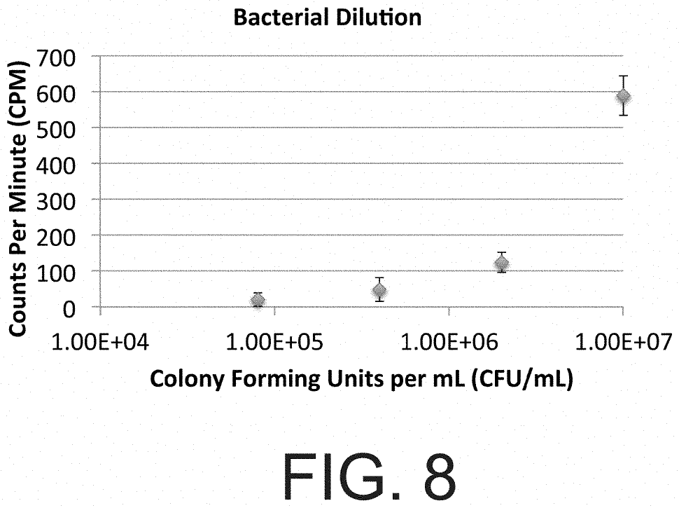

There was greater uptake with higher concentrations of bacteria, suggesting that [¹¹C]TMP may quantitatively report the number of bacteria in a given infection. FIG. 8 shows that the more live bacteria that are present the greater the binding/signal of [¹¹C]TMP.

### Example 16: ¹⁸F-FP-TMP Uptake/Specificity Studies

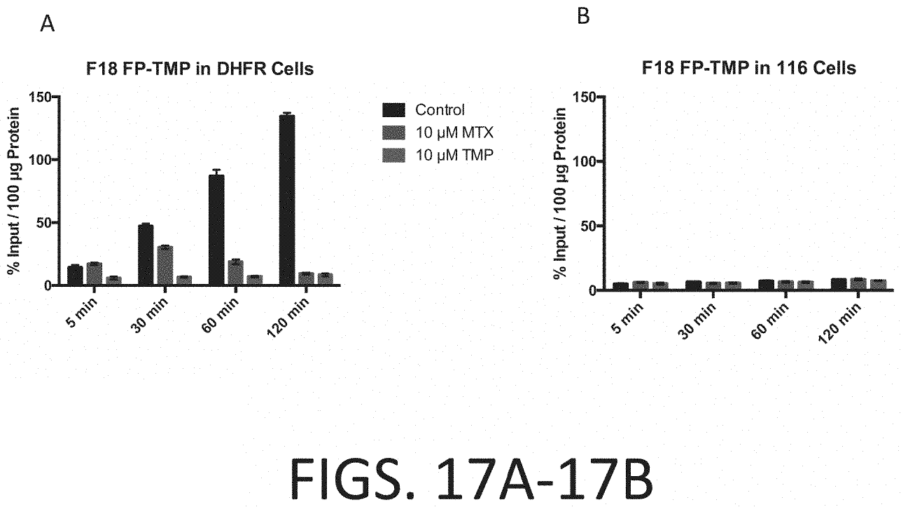

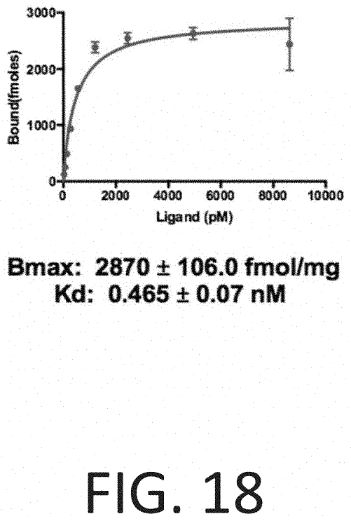

¹⁸F-FP-TMP uptake/specificity studies in HCT116 and HCT116 DHFR cells were conducted. Forty thousand cells/well/100 μL were plated in a 96 well plate 24 hours prior to the experiment. On the day of the experiment, about 70,000 cpm/50 μL/well in DMEM media were added and incubated for about 60 minutes at about 37° C. Fresh MTX and TMP (10 μM) solutions were prepared and then co-incubated with ligand. At the end of each time, media was aspirated and individual wells were counted in a gamma counter. See, Tables 1 and 2 and FIGS. 17 and 18.

TABLE 1

| | DHFR | | 10 μM MTX | | 10 μM TMP | |
|---|---|---|---|---|---|---|
| Time (min) | Mean | SD | Mean | SD | Mean | SD |
| 5 | 14.3 | 1.87 | 17.2 | 1.03 | 5.82 | 1.1 |
| 30 | 47.2 | 1.84 | 30.4 | 1.29 | 6.66 | 0.48 |
| 60 | 87.1 | 4.82 | 18.8 | 1.84 | 7.04 | 0.7 |
| 120 | 134.6 | 2.71 | 9.42 | 0.73 | 8.38 | 1.13 |

TABLE 2

| | Control | | 10 μM MTX | | 10 μM TMP | |
|---|---|---|---|---|---|---|
| Time (min) | Mean | SD | Mean | SD | Mean | SD |
| 5 | 5.11 | 0.49 | 6.32 | 0.41 | 5.37 | 0.78 |
| 30 | 6.58 | 0.51 | 5.59 | 0.43 | 5.83 | 0.48 |
| 60 | 7.37 | 0.62 | 6.74 | 0.63 | 6.48 | 0.52 |
| 120 | 8.4 | 0.52 | 8.62 | 0.66 | 7.61 | 0.33 |

The Lowry method was then conducted for protein determination (DHFR: 22.3 μg and 116: 24.5 μg).

These results illustrate that there was over 7-fold induction and 15-fold signal induction in DHFR cells compared to control cells at 30 and 120 minutes respectively. This is an improved signal to noise (target to background) compared to [¹¹C]TMP

The B-Max and kD for [¹⁸F]FPTMP were 2870 and fmol/mg and 0.465 nM respectively. This indicates a very strong binding affinity of [¹⁸F]FPTMP for Ec DHFR in mammalian cell. This affinity is on par with unmodified TMP and suggest excellent targeting of the desired protein and little if any off target effects or binding.

### Example 17: [¹⁸F]FPTMP In Vitro Detection

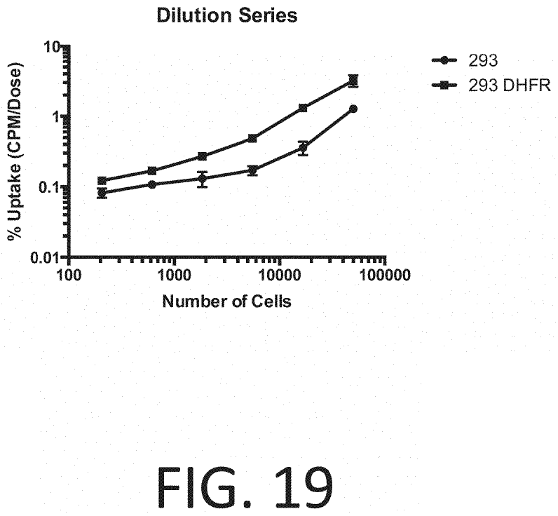

Serial Dilutions of 293 and 293 DHFR cells were incubated with [¹⁸F]FPTMP for 2 hours and washed twice. Cellular uptake was assessed on a gamma counter (FIG. 19).

These results show that [¹⁸F]FPTMP in vitro detection is sensitive to a few hundred DHFR cells.

### Example 18: [¹⁸F]FPTMP Validation: Tumor Model

Small animal micro PET/CT of DHFR tumors with [¹⁸F]FPTMP. HCT116 tumors were xenografted subcutaneously (8 million cells) to the shoulders of nude mice. The tumors were grown for 14 days and imaged using small animal PET followed by CT imaging. A representative animal is shown at imaging time point 4 h after [¹⁸F]FPTMP ˜0.1 mCi IV.

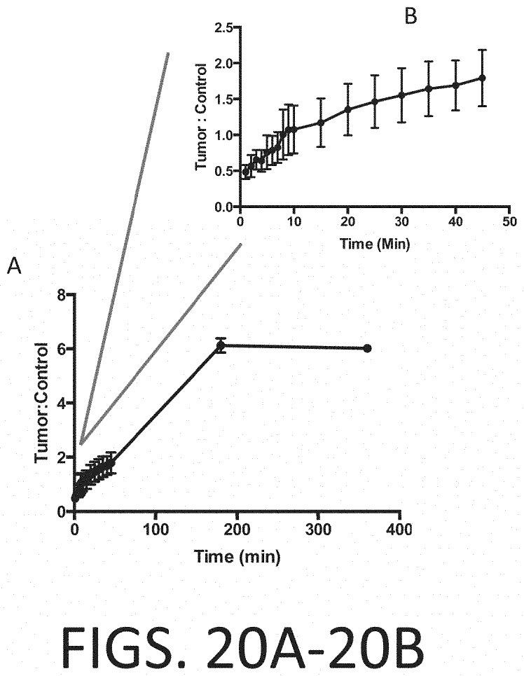

FIGS. 20A and 20B illustrate the quantification of in vivo uptake data. Error bars represent standard deviation (n=3).

### Example 19: [¹⁸F]FPTMP Bio-Distribution

Bio-distribution studies were performed at 6 h after ¹⁸F FPTMP injection.

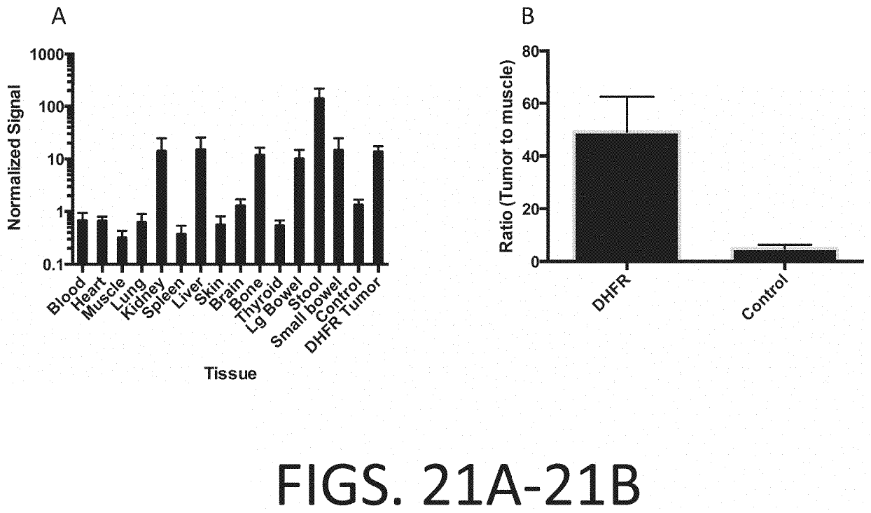

FIGS. 21A/B provide the quantification of tumor to muscle ratio. Error bars represent standard deviation (n=3).

### Example 20: [¹⁸F]FPTMP Bacterial Uptake In Vitro

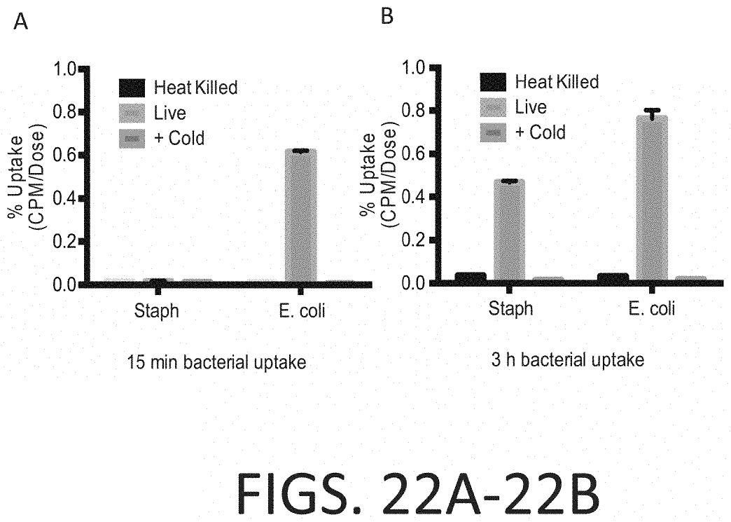

Heat-killed S. aureus and E. coli bacteria were boiled at 98° C. for 10 minutes. The bacteria were then pelleted at 6000 g for 5 minutes and incubated with [¹⁸F]FPTMP in PBS for 15 min and 3 h at 37° C. with and without cold TMP (10 μM). The bacteria were pelleted repeatedly, washed twice with cold PBS, and assayed for uptake with a gamma counter. Error bars represent standard deviation (n=3). See, FIGS. 22A/B.

### Example 21: Infection v Inflammation v Tumor Using [¹⁸F]FPTMP

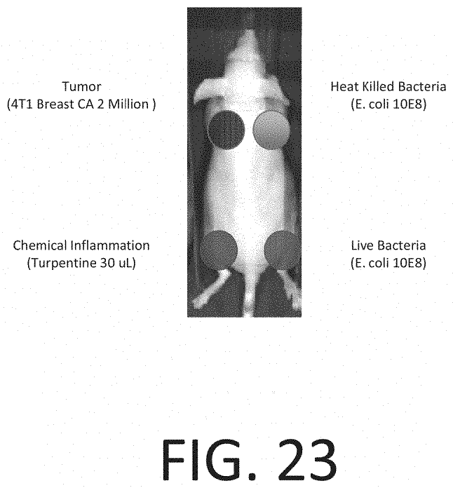

Balb/c mice were injected with 30 μL of turpentine into their left leg 3 days prior to imaging. This provided a bland (non-infectious) inflammation control. Live bacteria (E. coli, 1×10⁸ CFU) were injected subcutaneously into the right leg and heat killed bacteria were injected into the right shoulder 12 h prior to imaging. Mouse breast cancer cells (4T1, 2 million) were injected subcutaneously into the left shoulder 12 h prior to imaging (FIG. 23).

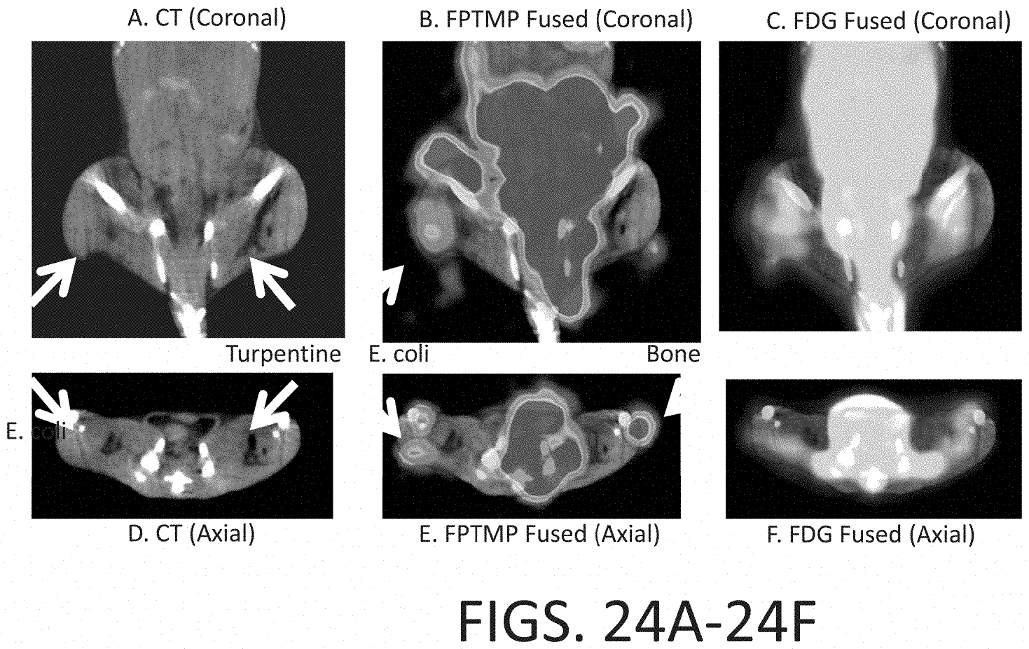

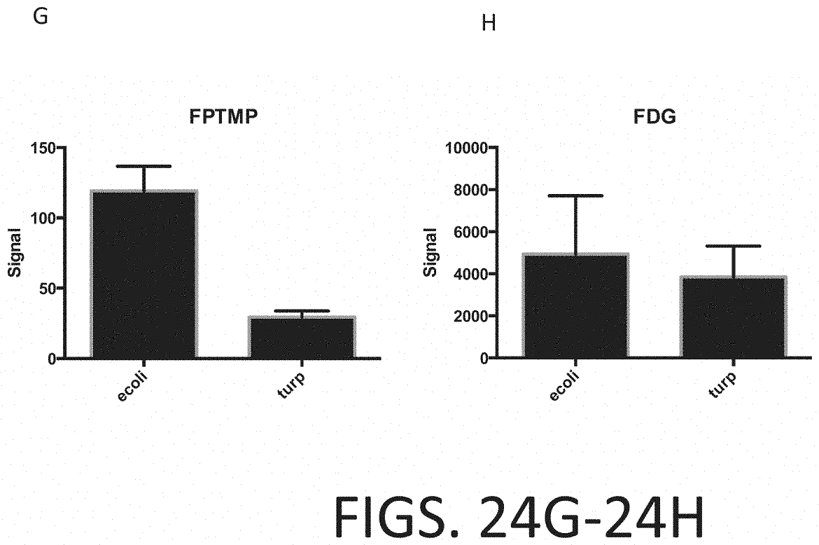

Balb/c mice were injected with 1 mg of D-luciferin prior bioluminescence imaging. This effectively checked for live 4T1 tumor cells (carrying luciferase gene). The E. coli contain the Lux operon (Bioluminescence protein and substrate synthetic pathway) and are bioluminescent without exogenous luciferin. Bioluminescent imaging was performed with 5 minute bin prior to radiotracer injection (FIGS. 24A-24E).

Balb/c mice were injected with about 100 mCi of [¹⁸F]FPTMP. Time activity curves of the mice were performed over 45 minutes. Subsequent time points at 2, 4, and 6 hours were performed (n=3). The following day animals were injected with about 200 mCi of fludeoxyglucose ([¹⁸F]FDG). Animals were sacrificed, tissues harvested for Bio-D and lower limbs were OCT flash frozen for future IHC (FIGS. 25A-25F).

These results illustrate that [¹⁸F]FPTMP uptake was observed in live bacteria, but not in turpentine inflammation and that there was no difference in [¹⁸F]FDG uptake for bacteria versus turpentine inflammation (FIGS. 24A-24H). These results also illustrate that there is no [¹⁸F]FPTMP uptake in tumor cells (FIGS. 25A-25F).

### Example 22: Analysis of Experimental Results

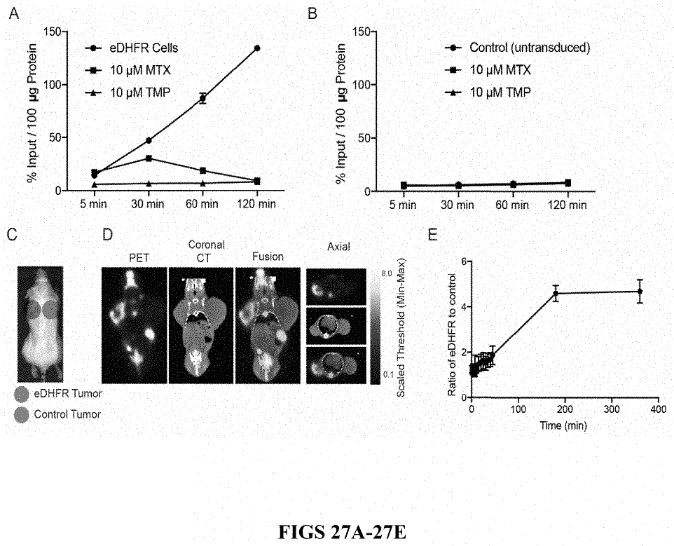

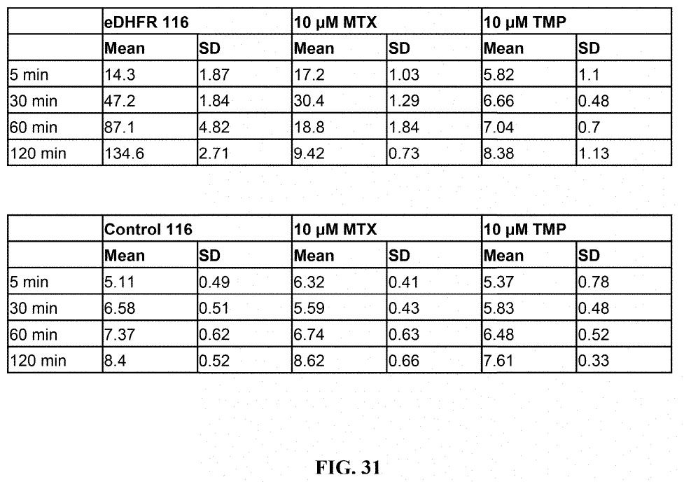

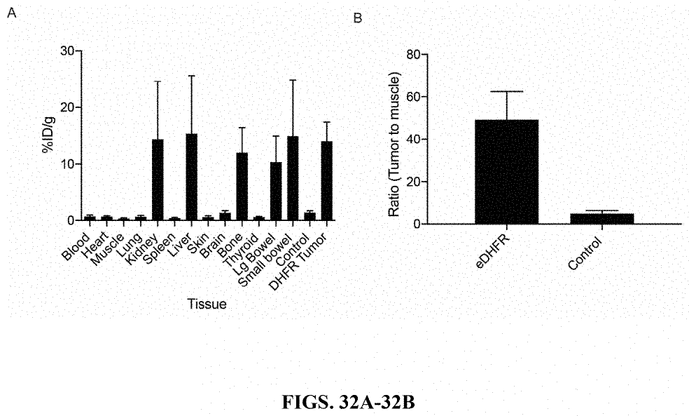

To test the ability of [¹⁸F]FPTMP to enter mammalian cells and bind bacterial eDHFR, a cell uptake experiment was performed. Previously derived HCT116 expressing eDHFR-YFP and control untransduced HCT116 cells were grown overnight in a 96-well plate.¹³ [¹⁸F]FPTMP was synthesized as previously described with high specific activity (between 5,000 and 15,000 Ci/mmol) and radiochemical purity (99%).¹⁴ The cells were then incubated with [¹⁸F]FPTMP with and without excess, competing, non-radioactive TMP (10 uM) or methotrexate (MTX, 10 uM). eDHFR cells showed a time dependent increase in uptake whereas there was no accumulation in control (non-transduced) cells. Excess TMP blocked binding completely and MTX, which is also a nM inhibitor of both mammalian and bacterial DHFR, blocked binding completely by 120 minutes (FIGS. 27A and 27B).^{16,17} Numerical values of uptake are shown in the supplemental information (FIG. 31).

Given rapid, high-level, and specific [¹⁸F]FPTMP uptake in vitro, these same cell lines (HCT116 control and eDFHR-YFP) were xenografted on the shoulders of CD-1 nu/nu mice and were grown over 3 weeks (FIG. 27C). [¹⁸F]FPTMP was administered by tail vein (˜100 □Ci/mouse) and each mouse was imaged dynamically for 45 minutes with PET; static PET images were acquired 3 h and 6 h after injection (FIGS. 27D and 27E). Ex vivo biodistribution analysis of [¹⁸F]FPTMP after the final PET/CT imaging time point demonstrated marked uptake in eDHFR tumors compared to control tumors (FIG. 31A) and over 40-fold increased uptake in eDHFR tumors compared to muscle (FIG. 31B). Normal tissues that had very low level of uptake include the blood, heart, lungs, muscle, spleen, skin, and brain. Tissues that showed high uptake and/or excretion of the tracer include the kidney, liver, and bowel. Uptake in the bone has been shown to be in part due to defluorination of [¹⁸F]FPTMP, a common occurrence in rodent models of fluorinated radiotracers that is not seen in non-human primates.¹⁴

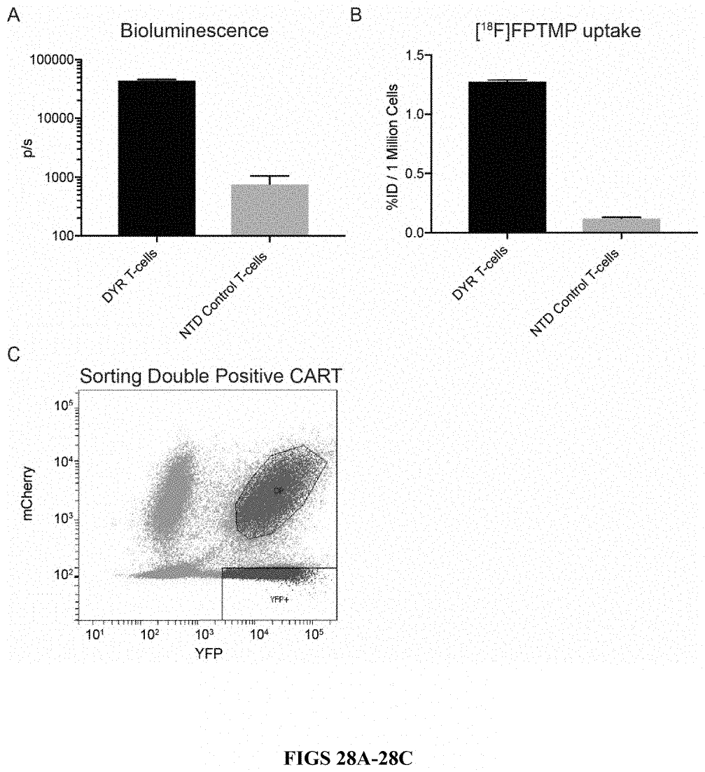

Given these findings in tumor cells, this strategy was next evaluated in imaging primary human T cells. Renilla renformis luciferase (DYR plasmid) was added to this construct, separated by a T2A ribosomal cleavage site to create a triple reporter plasmid referred to as the DYR plasmid (eDHFR-YFP-Renilla, FIG. 26C). The luciferase served as an imaging approach known to work well in small animals for identifying the location of live engineered cells. Lentivirus-transduced primary human T cells were sorted on YFP expression. An initial in vitro test of the bioluminescence and PET reporters in the sorted T cells was performed. There was over a 40-fold increase in BLI signal from the DYR T cells in comparison to the non-transduced (NTD) T cells following co-incubation with coelanterazine bioluminescence substrate (FIG. 28A). Sorted DYR T cells demonstrated an approximately 10-fold increase in [¹⁸F]FPTMP uptake relative to control NTD T cells following a 30 minutes incubation. (FIG. 28B).

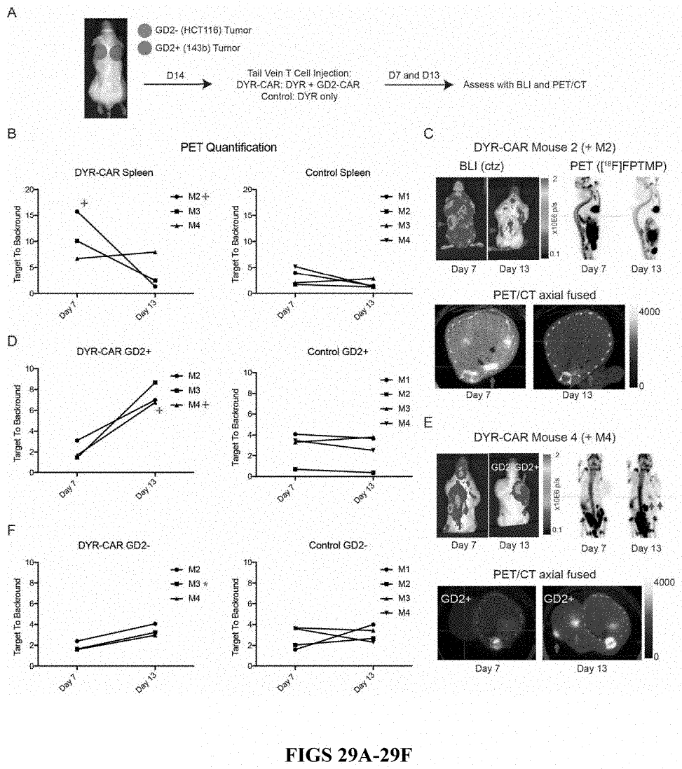

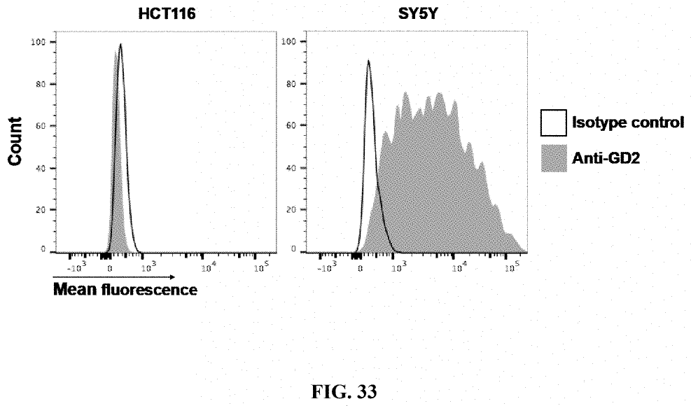

To evaluate CAR T cell trafficking in live animals, the presently disclosed triple imaging reporter was applied to a known CAR T cell system targeting GD2. Primary human T cells were co-transduced to express DYR and a high-affinity variant of a 4-1BB-based anti-GD2 CAR (GD2-E101K) containing an mCherry fluorescent protein separated by a T2A site.¹⁵ Transduced T cells (DYR-CAR) were sorted on YFP and mCherry to isolate double positive DYR-CAR or single positive control DYR-only T cells (FIG. 28C). A rodent model was designed to evaluate the trafficking of DYR-CAR T cells to GD2+143b human osteosarcoma subcutaneous xenograft tumors and used a GD2− control tumor, HCT116 human colon carcinoma (FIG. 29A). HCT116 tumors were confirmed to be GD2 negative with flow cytometry (FIG. 33). GD2+ and GD− tumors were grown for 14 days on the opposite shoulders of recipient mice prior to T cell injection. The treatment group received the double positive DYR-CAR T cells while the control group received DYR only T cells by tail vein injection. Mice were imaged first by BLI with coelenterazine injection followed by PET/CT with [¹⁸F]FPTMP injection on days 7 and 13 after T cell administration. Mouse 1 of the DYR-CAR group died during a PET/CT imaging session—likely from anesthesia and was excluded from the analysis.

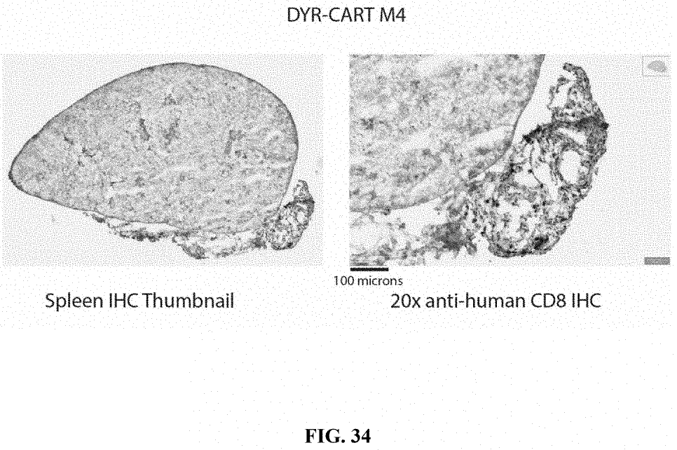

The spleens of DYR-CAR mice M2 and M3 demonstrated increased signal on day 7 and that decreased by day 13 in both optical and PET modalities (FIGS. 29B-29C). The M4 mouse maintained a low level of signal near the spleen which was corroborated ex vivo IHC (FIG. 34). Control mice showed no significant uptake in the spleen and basal levels on day 7 or 13. Representative DYR-CAR mouse M2 images demonstrate increased signal in the spleen on day 7 that decreased by day 13.

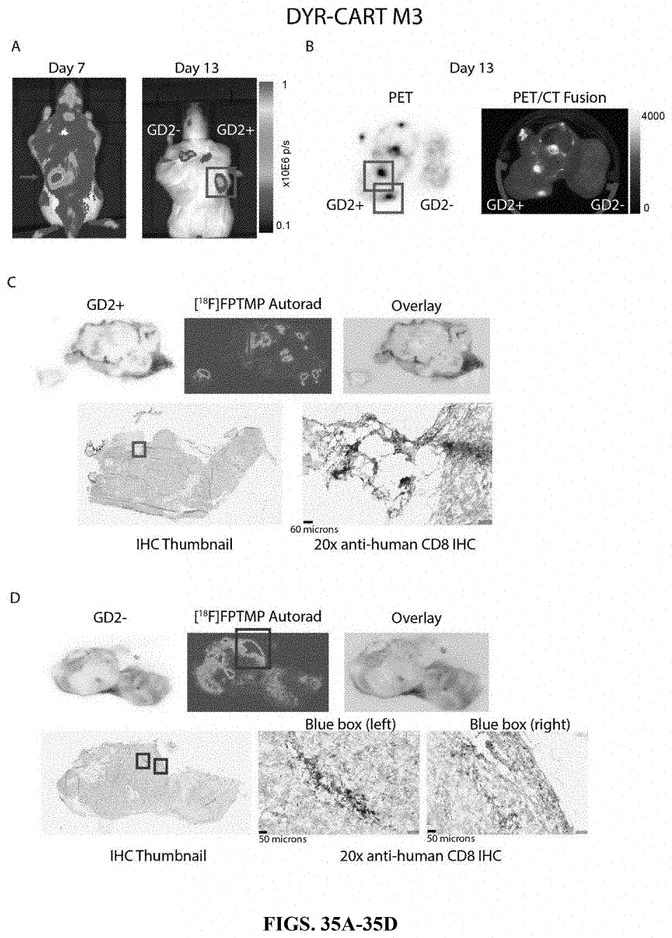

As splenic signal decreased over time, foci of increased signal developed within the GD2+ tumors using both optical and PET modalities (FIGS. 29D-29E and FIGS. 35A-35B). These foci were non-uniform and distributed about different locations in the tumor. At these early time points, the foci were generally peripheral, located medially against the chest wall or superficially invading into the tumor just beneath the dermis. The number and extent of the CAR T cell foci were seen to better advantage in rotating 3D maximum intensity projections at the day 13 time point. For quantification, regions of interest were drawn around the entire tumors, and the signal maximum in the tumors was divided by the signal maximum from the heart/mediastinal blood pool signal, thereby yielding a target to background ratio. Target to background in GD2+ tumors reached 6-8-fold increased signal, whereas in control tumors, target to background ratios were below 4-fold. Importantly, foci of [¹⁸F]FPTMP uptake were easily distinguished within the GD2+ tumors and in comparison to normal tissues. The GD2− control tumors did not demonstrate similar foci of increased uptake by PET imaging following treatment with DYR-CAR T cells (FIG. 29F and FIG. 35B).

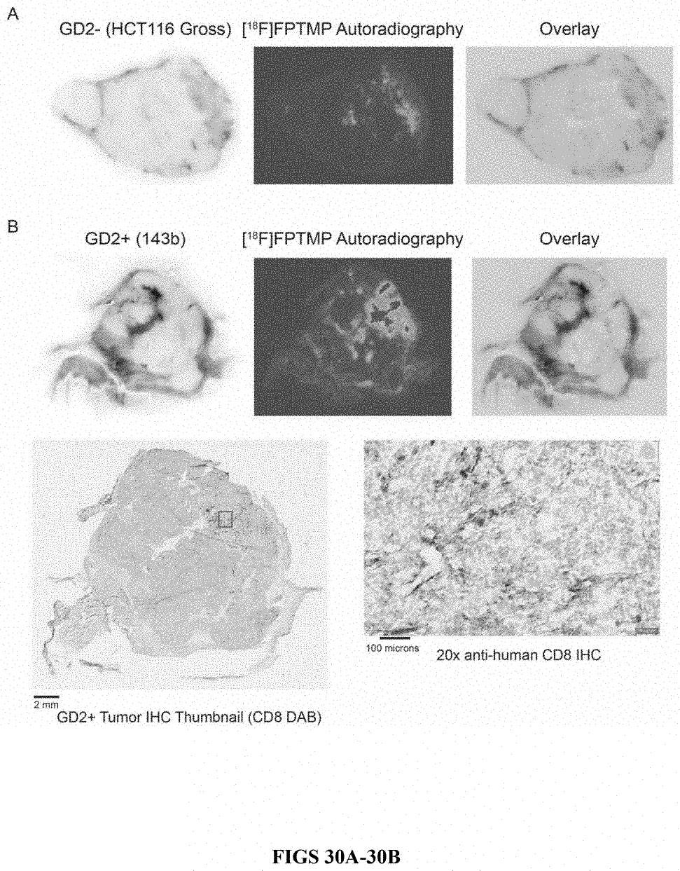

To validate that the foci of increased signal on BLI and PET imaging were indeed coming from DYR-CAR T cells, an autoradiography was performed on representative mouse spleens and tumors. For example, DYR-CAR mouse M4, which showed persistent uptake in the region of the spleen on PET imaging, also showed positive staining for anti-human CD8 cells in the spleen (FIG. 29B and FIG. 34). As expected in, there was no autoradiography signal above background in the DYR-CAR mouse M4 GD2− tumor (FIG. 30A); whereas there was clear correlation between the imaging, the autoradiography signal, and anti-CD8 IHC in the GD2+ tumor (FIG. 30B).

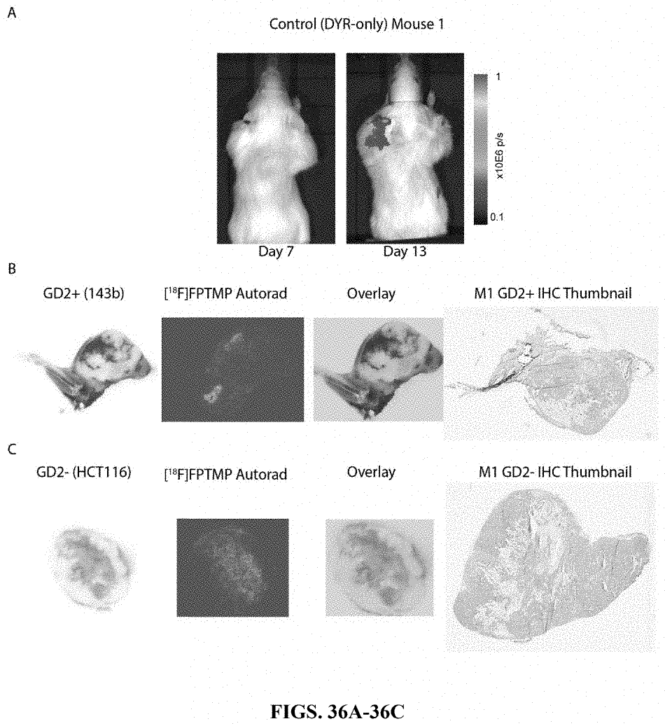

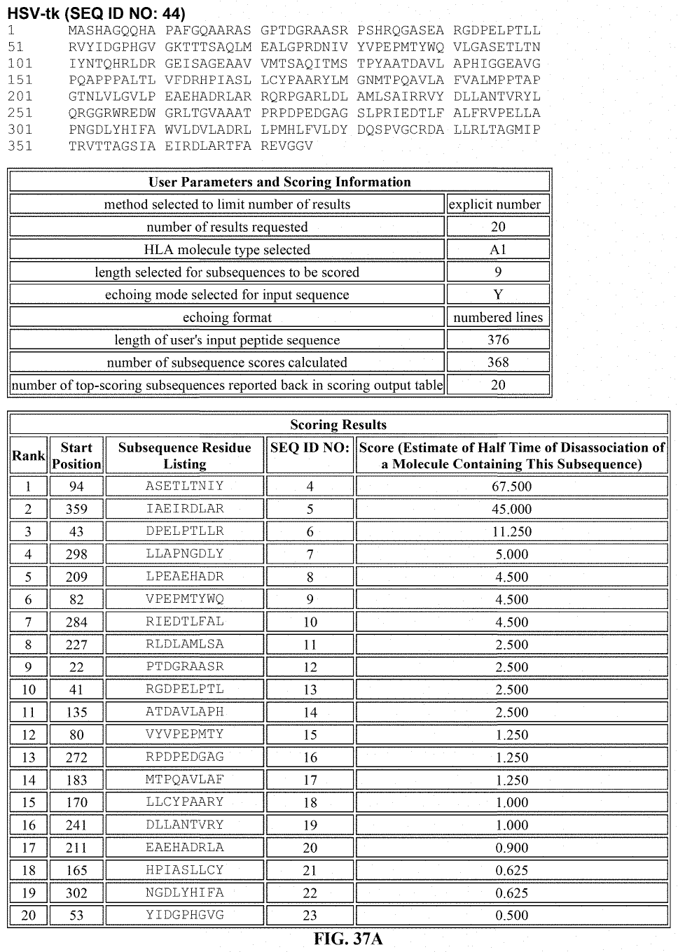

Although there was no significant focal PET imaging signal from the GD2− tumor in M3 of the DYR-CAR group, there were a few superior foci of BLI signal within the tumor. This area correlated with moderately increased signal on autoradiography and indeed positive CD8 T cells were noted on IHC (FIGS. 35B and 35D). Control DYR only T cells, did not show significant localization by BLI or PET imaging, nor was there any focal signal found by autoradiography or IHC (FIGS. 36A-36C).

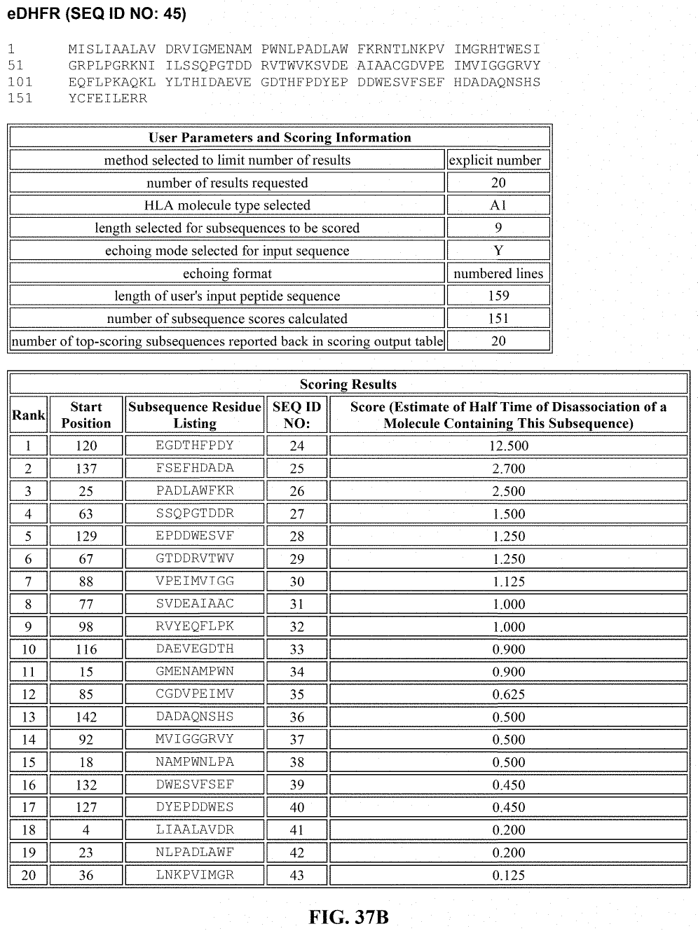

Finally, to provide in silico evidence for the comparative potential for immunogenicity between HSV-tk and eDHFR, representative HLA peptide motif searches were performed and the estimated half-time of dissociation with HLA-A1 showed 16 sequences for HSV-tk>1.0 (max half time of dissociation 67.5 s), whereas eDHFR had 9 such sequences (max half time of dissociation 12.5 s, FIGS. 37A-37B.¹⁸

### Example 23: Discussion

Prior to this work, this strategy using a carbon-11 version of trimethoprim ([¹¹C]TMP) was presented in a simple rodent xenograft model.¹³ While the molecular similarity of [¹¹C]TMP to the clinically available therapeutic antibiotic has allowed to rapidly test the biodistribution and estimated organ dosimetry in humans, Fluorine-18 is more practical for human imaging given its longer half-life and energetics. The first synthesis of [¹⁸F]FPTMP, and promising non-human primate biodistribution recently was published in a complementary application of these radiotracers for bacterial infection imaging, and the use of [¹⁸F]FPTMP in engineered human cells was a natural next step building upon the introductory work above.¹⁴

The uptake of [¹⁸F]FPTMP in HCT116 cells carrying eDHFR compared to untransduced cells was greater than 15-fold, and demonstrated time dependent accumulation. As expected, excess unlabeled TMP and MTX were able to compete with [¹⁸F]FPTMP in eDHFR cells (FIGS. 27A-27B). Taking these same cell lines into a xenograft rodent model, there was over a 4-fold difference in [¹⁸F]FPTMP uptake between eDFHR tumor and untransduced control tumor using a ratio of the maximum counts from each tumor (FIGS. 27C-27E). The ex vivo biodistribution suggested low background in many important tissues, for example blood, muscle, lung, and brain, and the uptake ratio of eDHFR tumor to muscle was over 40-fold (FIGS. 31A-31B), supporting the ability of this system to serve as a sensitive tool for detecting cells that carry the eDHFR reporter gene.

A more difficult challenge for a PET reporter gene strategy is imaging systemically delivered cells, where cell numbers in a particular tissue or imaging voxel are often relatively low. The characterization of the targeting of CAR T cells to GD2 disialoganglioside was performed for the treatment of pediatric cancers such as neuroblastoma or osteosarcoma.^{15,23} A triple reporter imaging construct that fused eDHFR-YFP with a T2A cleavage site followed by Renilla luciferase (DYR) was constructed herein (FIG. 26C). After lentiviral transduction of primary human T cells, the bioluminescent signal and uptake of [¹⁸F]FPTMP in transduced compared to untransduced T cells was tested, showing over 40-fold bioluminescent signal induction and 10-fold radiosignal induction (FIGS. 28A-28B). The bioluminescent signal provided supporting evidence for the trafficking of DYR-CAR T cells to the spleen and GD2+ tumors, and a handle for the optimal timing of the more operationally intensive tracer synthesis and PET/CT imaging.

Immunodeficient, humanized NSG mice were xenografted with GD2+ osteosarcoma 143b cells (10 million) on one shoulder with GD2− human colon carcinoma HCT116 cells (10 million) on the contralateral shoulder. The tumors grew for two weeks to similar sizes and were sufficiently large to be vascularized. DYR CAR T cells or control DYR only T cells were injected by tail vein. As expected, there was early CAR T cell signal in the spleen suggesting that the spleen is reservoir for GD2 CAR T cell residency or expansion, consistent with prior observations (FIG. 29B).¹⁵ As the splenic signal both by BLI and PET dropped in several mice, there were increases in signal coming preferentially from the GD2+143b tumors (FIG. 29D), suggesting trafficking to epitope containing tissues by approximately 2 weeks. It was found that in one GD2− HCT116 to control tumor there was mild generalized uptake on PET imaging which could represent a component of alloreactivity, and that there were several foci of BLI signal that appeared to correlate with a collection of DYR-CAR T cells within that particular sample on IHC (FIGS. 35A-35D). There was no clear trend for this across other mice. Also, there was no significant signal from the DYR control T cell injected mice, suggesting that without the CAR, there was no proliferation and thus the T cells died (FIGS. 29A-29F and FIGS. 35A-35D).

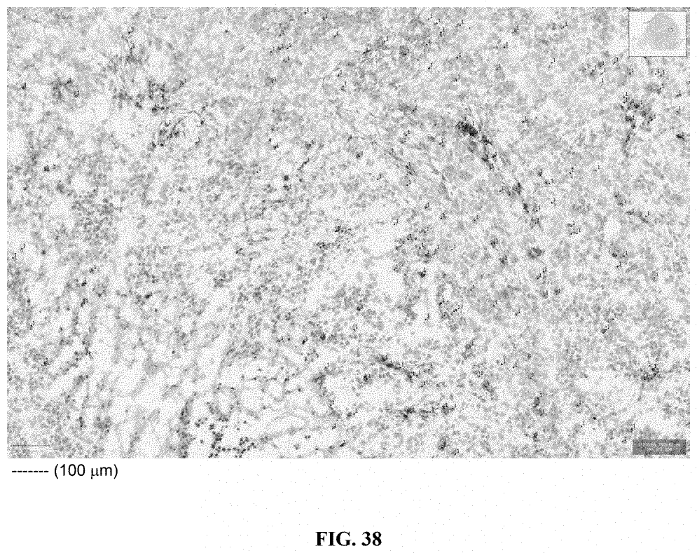

A key component for clinical utility of this imaging strategy is the sensitivity of detection in terms of number of cells in a particular tissue. For example, if approximately 0.6-6×E8 cells are injected in the case of tisagenlecleucel, a CART19 therapy (package insert), related questions arise: How many cells need to reach the target tissue for efficacy? What are the off-tumor sites of accumulation/toxicity? As few as 11,000 CD8 DYR-CAR T cells per mm³ can be detected in tumor tissues, a sensitivity that is comparable or better than other PET reporter techniques including human-neuroendocrine (hNET) and HSV-tk recently benchmarked in a direct T cell injection approach (FIG. 38).⁹ CD8 rather than CD4 T cells are the dominant tumor infiltrating population in this CAR T model. ¹⁵ Additionally, this value supports and improves upon empirically derived number of detectable cells in a xenograft model using the related radiotracer [¹¹C]TMP, which has a shorter isotope half-life and theoretically lower target to background ratio.¹³

The ability to monitor the CAR T cell trafficking over time was disclosed herein, for example in the spleen at early time points and later at the tumor, suggesting that this approach allows long-term (weeks-months) monitoring of engineered cells—a key feature over direct labeling techniques such as Zr-89 or In-111 oxime.^{24,25}

In silico evidence suggests that in comparison to HSV-tk, eDHFR has fewer potential immunogenic peptides and that of the possible peptides, eDHFR peptides show decreased half-times of dissociation with a representative human HLA (FIGS. 37A-37B).¹⁸

### Example 24: Conclusions

It was tested herein the ability of [¹⁸F]FPTMP, a fluorine-18 PET probe, to image primary human CAR T cells engineered to expressed the PET reporter gene eDHFR, yellow fluorescent protein (YFP), and Renilla luciferase (rLuc). Engineered T cells showed approximately 50-fold increased bioluminescent imaging (BLI) signal and 10-fold increased [¹⁸F]FPTMP uptake compared to controls in vitro. eDHFR-expressing anti-GD2 CAR T cells were then injected into live mice bearing control GD2− tumor on one shoulder and GD2+ tumor on the other. PET/CT images acquired on days 7 and 13 demonstrated early residency of CAR T cells in the spleen followed by on-target redistribution to the GD2+ tumors. This was corroborated by ex vivo autoradiography and anti-human CD8 immunohistochemistry. Surprisingly, a high sensitivity of detection was found for infiltrating CD8 CAR T cells into the GD2+ tumor that is likely to be clinically relevant, ˜10×E4 cells per mm³. Taken together, these data suggest that the [¹⁸F]FPTMP/eDHFR PET probe/reporter gene pair offers important advantages including genetic portability, facile radiosynthesis, favorable biodistribution and kinetics, and high sensitivity, that could better allow investigators to monitor immune cell trafficking to tumors.

Disclosed herein is a PET imaging strategy to monitor CAR T cells using [¹⁸F]FPTMP and eDHFR as a reporter gene. The ability to image small numbers of transgenic cells in humans would be helpful to accelerate new cell-based therapies into the clinic, and may provide a better understanding of treatment success, failure, and toxicity.

### Example 25: Novel PET Radiotracers for Imaging CAR T Cells with a Dual Function Report Gene

A dual function reporter gene that serves as both an imaging agent and a potent suicide gene could find widespread application as a safety switch in cell-based therapies, which would open the door to numerous imaging applications. Since CAR T cell therapy and other gene therapies carry significant risk, which includes cytokine release syndrome, neurological toxicity, on-target/off-tumor toxicity, insertional oncogenesis, graft versus host disease, and off-target antigen recognition, the elegance of a dual function suicide-reporter is key from both a regulatory and patient perspective.

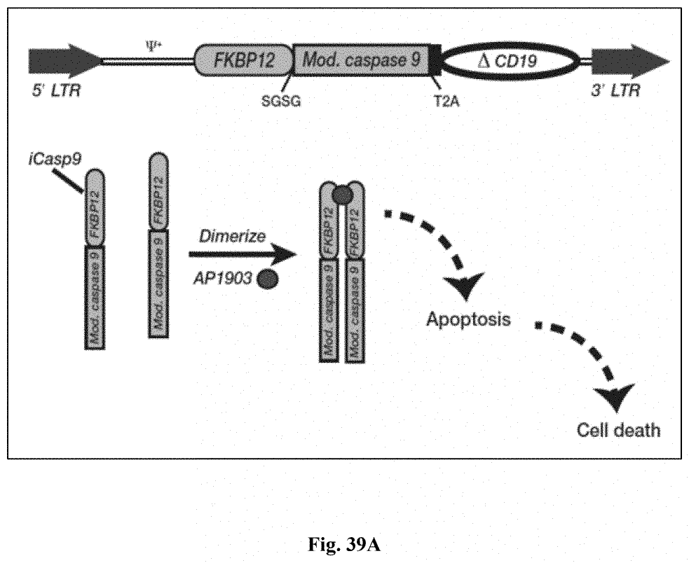

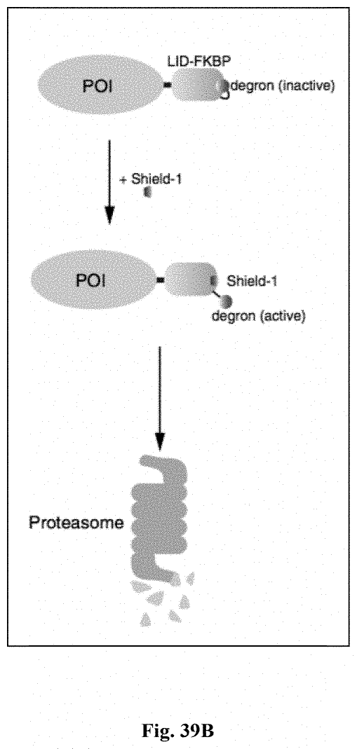

This disclosure contemplates the use of the mutant human FK506 binding protein, F36V-FKBP, as a dual function reporter gene, since it has a number of high affinity ligands such as Shield-1 that could serve as novel PET radiotracers, and it has been successfully utilized in humans as a component of the iCasp9 suicide gene (Di Stasi et al., N Engl J Med. 2011; 365(18):1673-1683). The iCasp9 system is based on human caspase 9, in which the recruitment domain of the caspase has been replaced by F36V-FKBP. This allows the caspase pathway to be activated by the small molecule AP1903, which is a dimer of Shield-1 that causes apoptosis via dimerization of the F36V-FKBP/caspase 9 fusion protein (FIG. 39A). iCasp9 has been used as a safety switch in patients that underwent stem-cell transplantation for relapsed acute leukemia, and a single dose of AP1903 was given to several patients who developed graft-versus-host disease (GVHD); more than 90% of the modified T cells were eliminated within 30 min after administration of AP1903, and the GVHD was terminated without abrogating immune reconstitution (Di Stasi et al., N Engl J Med. 2011; 365(18):1673-1683; Zhou et al., Blood. 2014; 123(25):3895-3905). Of note, the iCasp9 system has not been found to be immunogenic. Currently, the iCasp9 system is being utilized in several phase ½ clinical trials as a suicide gene for stem cell transplantation, as well as anti-GD2 CAR T cell therapy for neuroblastoma/sarcoma.

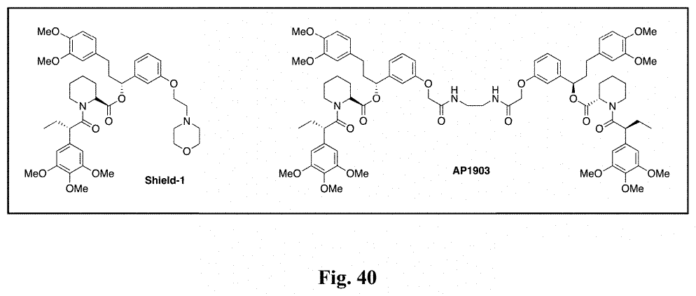

F36V-FKBP has also been used in the ligand-induced degradation (LID) system, in which protein expression can be controlled by the addition of Shield-1 (FIG. 39B) (Bonger et al., Nat Chem Biol. 2011; 7(8):531-537). The LID system is based on the addition of a degradation sequence (degron) to the C terminus of F36V-FKBP, which is then fused to a protein of interest. In the absence of Shield-1 the degron is bound to FKBP and the protein is stable. However, when Shield-1 is present, it binds tightly to FKBP, displaces the degron, and induces rapid degradation of the LID domain and the fused protein of interest. Although the LID system has not yet been tested in humans, it should be non-immunogenic given its similarity to the iCasp9 system. In preclinical studies the LID system can be used to control expression of the CAR in T cells in a dose-dependent manner using Shield-1, with an IC₅₀ in the nM range.

[¹¹C] or [¹⁸F] radiolabeled Shield-1 (Shld1) can serve as a novel PET radiotracer for imaging F36V-FKBP because it has a high affinity for the mutant protein with an IC₅₀ of 3.3 nM, it is specific for the mutant protein versus wild-type FKBP, and it is a monomeric version of AP1903 which has already been given to humans without any reported serious adverse events (FIG. 40). Ligands such as Shield-1 and AP1903 that possess a “bump” in the FKBP binding domain have been shown to bind more tightly to the mutant F36V-FKBP relative to the wild-type protein by greater than three orders of magnitude; thus binding of [¹¹C/¹⁸F]Shld1 to wild-type FKBP should be negligible (Clackson et al., Proc Natl Acad Sci USA. 1998; 95(18):10437-10442). Additionally, [¹¹C/¹⁸F]Shld1 has no effect on the CAR T cells, since the radiotracer is a monomer and only the dimer (AP1903) is capable of activating the caspase pathway.

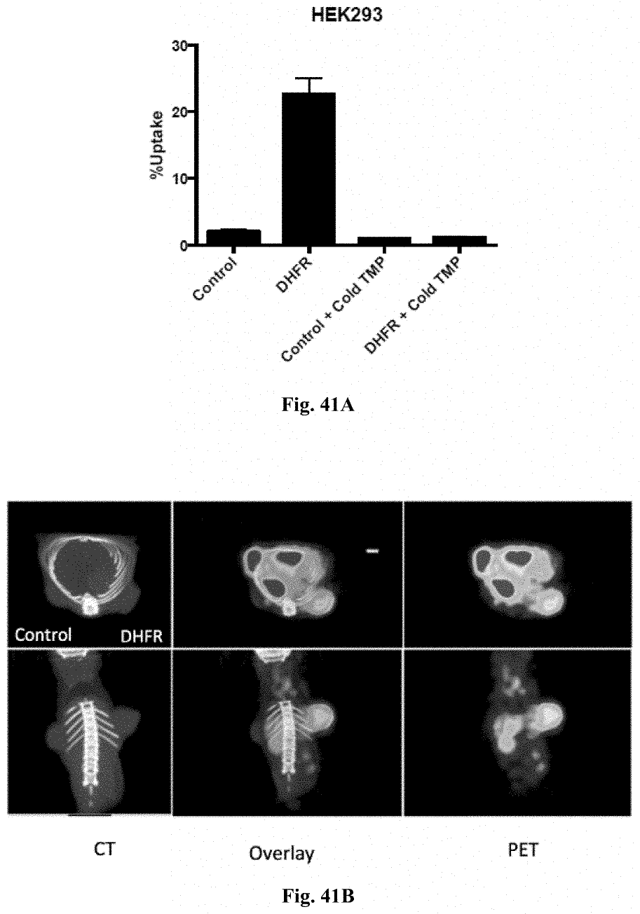

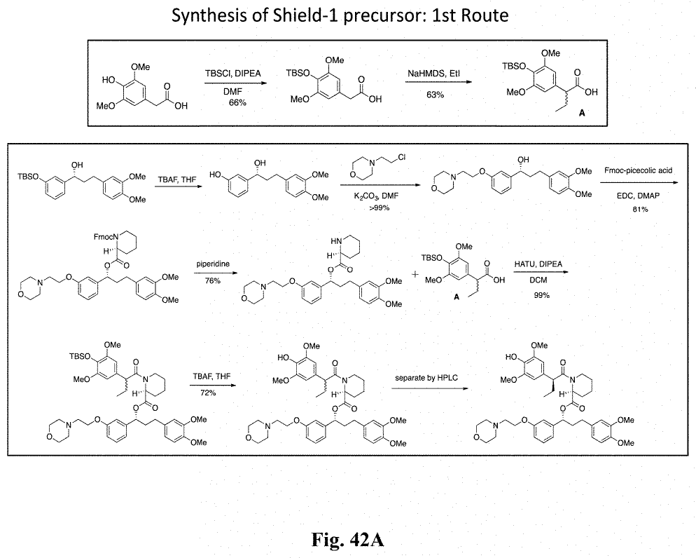

This disclosure also includes the use of a modified iCasp9 dual function suicide-reporter gene in which the ligand binding domain (F36V-FKBP) has been replaced by E. coli dihydrofolate reductase (eDHFR). [¹¹C] labeled trimethoprim (TMP) was previously synthesized as a novel PET radiotracer with high affinity for the DHFR enzyme. The novel PET radiotracer, [¹¹C]TMP, was synthesized by methylation with [¹¹C]methyl iodide; the desired product was isolated with high specific activity (500-1000 mCi/mmol) and 50-60% radiochemical yield. Cell uptake studies were then performed with HEK293 cells that had been transduced with the DHFR reporter gene, with untransduced HEK293 cells as a control; 10 uM cold TMP was also used as a control, to confirm that the uptake of [¹¹C]TMP was blocked in the presence of a large excess of cold TMP. The results are shown in FIG. 41A, in which the transduced HEK293 cells demonstrated 23% uptake of [¹¹C]TMP, versus less than 1-2% uptake in the controls. For the animal imaging experiments, DHFR positive and DHFR negative HCT116 tumors were xenografted subcutaneously (10 million cells) to the shoulders of nude mice. The tumors were grown for 10 days and imaged using small animal PET followed by CT imaging. A representative animal is shown at imaging time point 85-90 min after ˜1 mCi of [¹¹C]TMP injected IV through the tail vein (FIG. 41i). As can be seen in the images, the DHFR positive tumors demonstrated markedly increased radiotracer uptake relative to the control tumor (Sellmyer et al., Proc. Natl. Acad. Sci. U.S.A. 114: 8372-8377 (2017). Sellmyer et al., Mol Ther 25: 120-126 (2017).

The eDHFR/[¹¹C/¹⁸F]TMP system was shown to be highly sensitive for detecting transduced cells via PET imaging, and has only mild physiologic radiotracer uptake within the liver and GI tract. Since TMP is able to cross the blood-brain barrier (BBB), and is an established ligand for inducing protein dimerization, it has the potential to be an excellent PET radiotracer for a dual function suicide-reporter gene. Thus the eDHFR iCasp9 system is capable of being imaged with [¹⁸F]TMP.

This disclosure includes a novel dual function suicide-reporter gene that is capable of being imaged with a PET radiotracer and is also capable of serving as a potent suicide gene. This disclosure utilizes (1) the iCasp9 suicide gene that is currently in clinical use (based on F36V-FKBP/Shld1), and (2) the modified iCasp9 system based on eDHFR/TMP.

### Example 26: Synthesis and In Vitro Studies with [¹¹C]Shld1

[¹¹C]Shld1 was successfully synthesized, and demonstrated that the novel radiotracer is equivalent to commercially available cold Shld1 (FIGS. 4A-4B, 5A-5B). The Shld1 precursor was synthesized via a modified multi-step route (Amara et al., Proc Natl Acad Sci USA. 1997; 94(20):10618-10623; Yang et al., J Med Chem. 2000; 43(6):1135-1142). Methylation of the precursor with [¹¹C]methyl iodide yielded [¹¹C]Shld1 in 5% radiochemical yield (FIG. 43A) with a specific activity of >533 GBq/μmoL at end of bombardment. Analysis of the radiotracer by HPLC (FIG. 43B) demonstrated co-elution of [¹¹C]Shld1 at the same time as the reference standard; radiochemical purity is >95%. The functionality of the synthesized Shld1 was evaluated using HCT116 cells expressing luciferase fused to the FKBP12 L106P destabilizing domain (L106P-tsLuc). HCT116 cells treated with varying concentrations of synthesized Shld1 induced equivalent protein stabilization when compared to the literature (FIG. 5C) (Banaszynski et al., Nat Med. 2008; 14(10):1123-1127).

Cell uptake studies with [¹¹C]Shld-1 were performed with HEK293 and HCT116 cells that were transduced with the F36V-FKBP reporter gene (via a retroviral vector), with untransduced HEK293 and HCT116 cells as a control; FK506 (10 μM) was used as a blocking agent. Transduced HEK293 and HCT116 cells both demonstrated [¹¹C]Shld-1 uptake close to 100% input/100 g protein, at 40 min, with substantially less uptake in the untransduced cells and the blocking experiment (FIGS. 6A-6B).

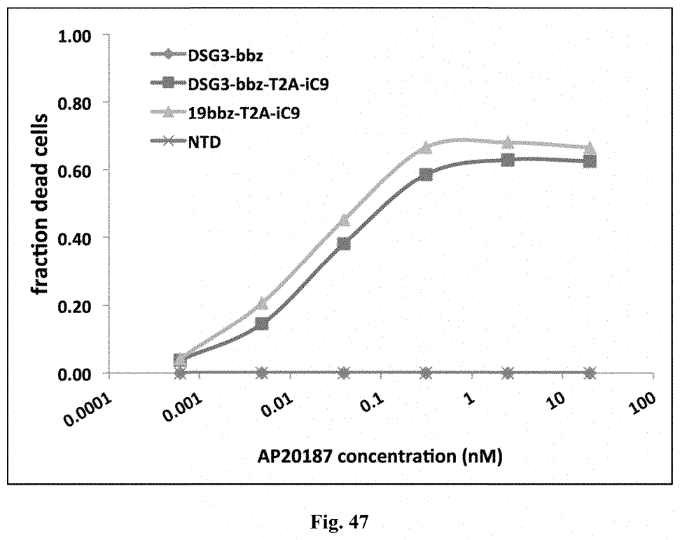

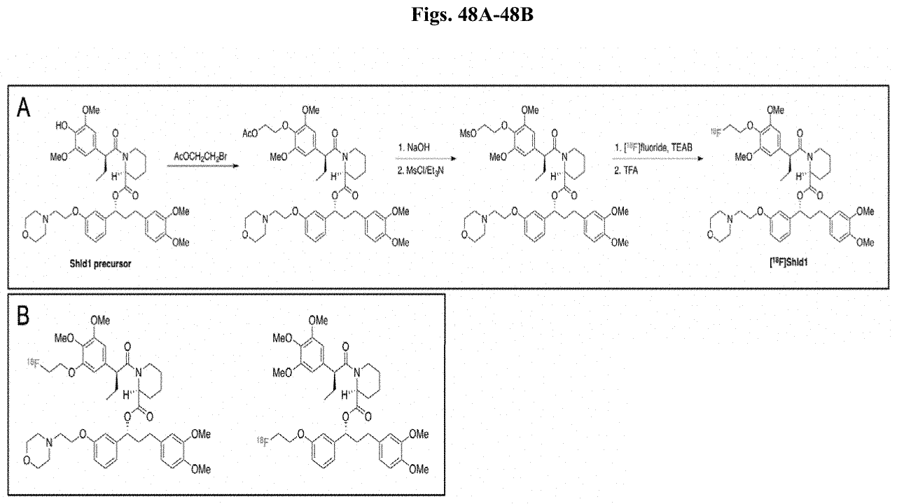

The iCasp9 system has been previously incorporated into CAR T cells directed against desmoglein-3 by others for studying pemphigus. T cells were transduced with two different CARs containing iCasp9 (iC9) and one CAR without iC9. The inducible caspase 9 was activated by treatment with varying concentrations of AP20187, a chemical inducer of dimerization which is an analog of AP1903 (FIG. 47). Of note is that the fraction of dead cells is less than 100% because not all of the cells were transduced. The IC₅₀ of AP20187 is <0.1 nM. Nontransduced cells (NTD) and cells without iC9 were not killed by AP20187.

### Example 27: Biodistribution of [¹¹C]Shld1 in Mice

Biodistribution studies were performed to determine the major organs of [¹¹C]Shld1 uptake, measure the rate of radiotracer clearance from the blood pool, and assess the ability of [¹¹C]Shld1 to cross the BBB.

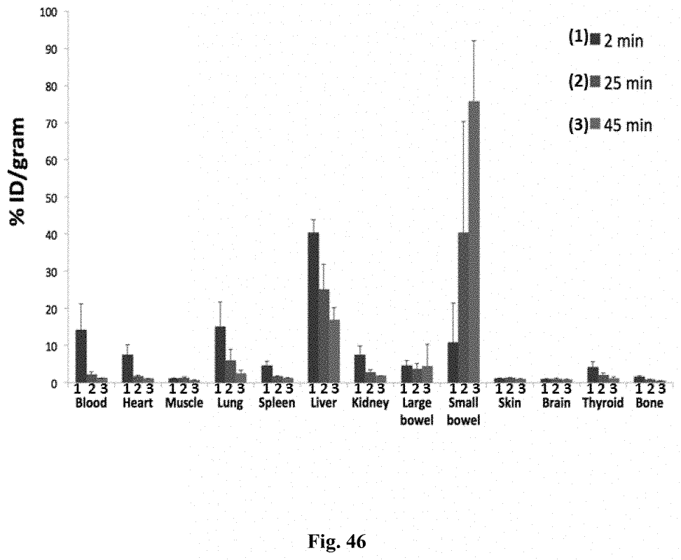

Biodistribution data for [¹¹C]Shld1 were collected at 2 min, 25 min, and 45 min following injection of radiotracer via the tail vein (50 μCi), with 4 mice (C57BL/6) per time point (FIG. 46). Organs were harvested and counted using a well gamma counter. Standard deviations were calculated for each sample.

### Example 28: Synthesis of FP Shield-1 and Comparison of its Affinity with Shield-1

Fluoropropyl-Shield-1 (FP-Shield-1) was synthesized from the Shield-1 precursor in one step (FIG. 59). The affinity of the synthesized FP-Shield-1 for F36V-FKBP was evaluated using HCT116 cells expressing luciferase fused to the FKBP12 L106P destabilizing domain (L106P-tsLuc). HCT116 cells were treated with increasing concentrations of synthesized FP-Shield-1 or commercially available Shield-1 (from Cheminpharma, LLC), and incubated with luciferin. The results demonstrated that the affinity of FP-Shield-1 for F36V-FKBP is similar to commercially available Shield-1. Thus, [¹⁸F]FP-Shield-1 should serve as an effective PET probe for imaging F36V-FKBP expression (including iCasp9 expression or LID expression) in vivo. Other [¹⁸F] labeled derivatives of Shield-1 (FIGS. 10A-10B) are also expected to bind to F36V-FKBP with high affinity. [¹⁸F] labeled derivatives of Shield-1 are expected to provide better image quality and sensitivity compared to [¹¹C]Shld1.

### Example 29: Development of a Modified iCasp9 Suicide Gene with eDHFR as the Ligand Binding Domain

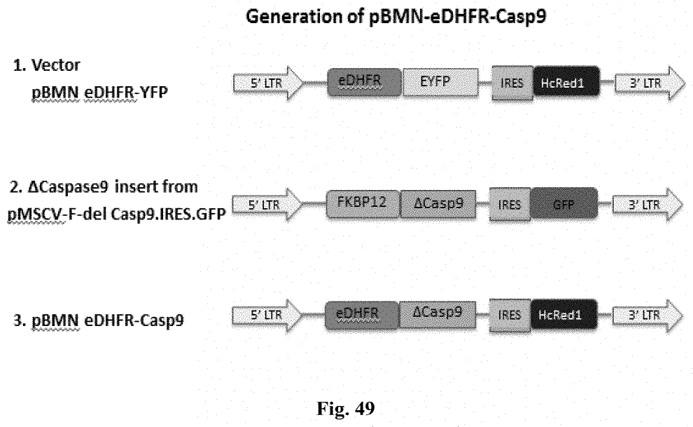

A modified iCasp9 dual function suicide-reporter gene in which the ligand binding domain (F36V-FKBP) has been replaced by eDHFR was generated (FIG. 49).

The eDHFR iCasp9 system was created by linking eDHFR to modified caspase 9 via a Ser-Gly-Gly-Gly-Ser (SEQ ID NO: 1) linker, as described in the literature for the F36V-FKBP component of the original iCasp9 system (Di Stasi et al., N Engl J Med. 2011; 365(18):1673-1683).

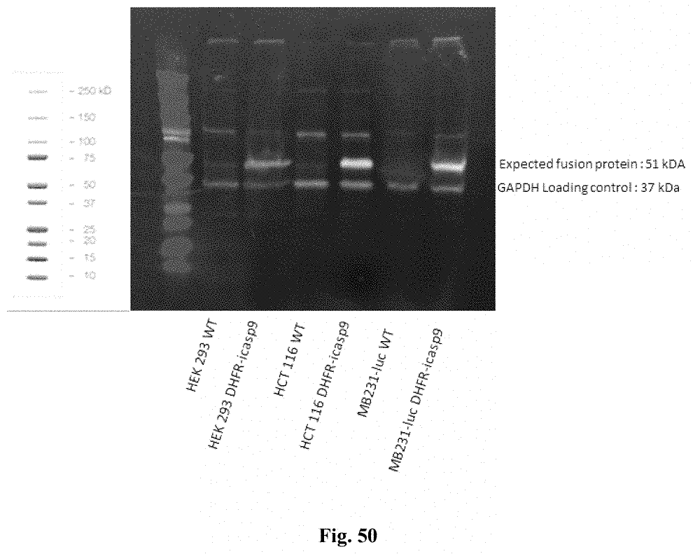

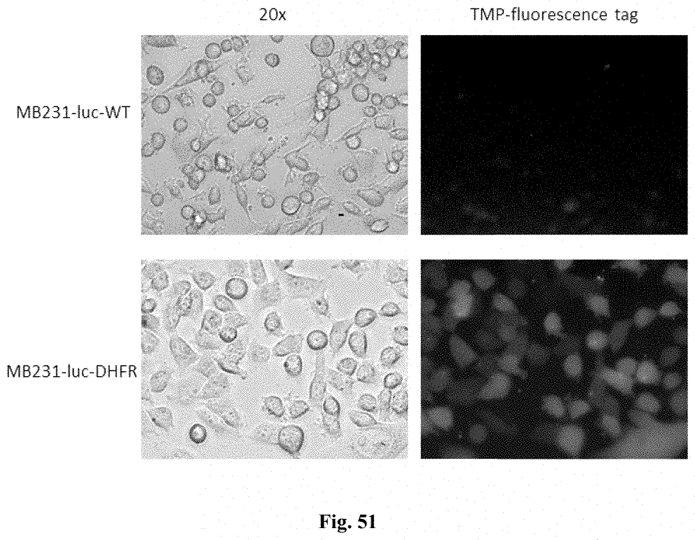

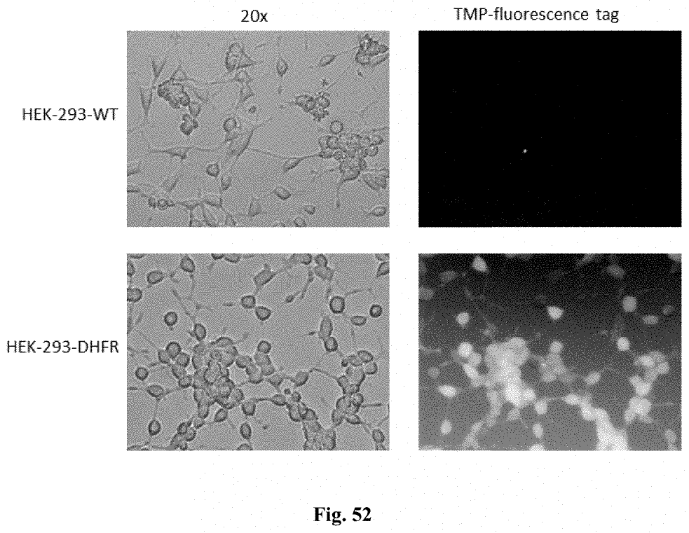

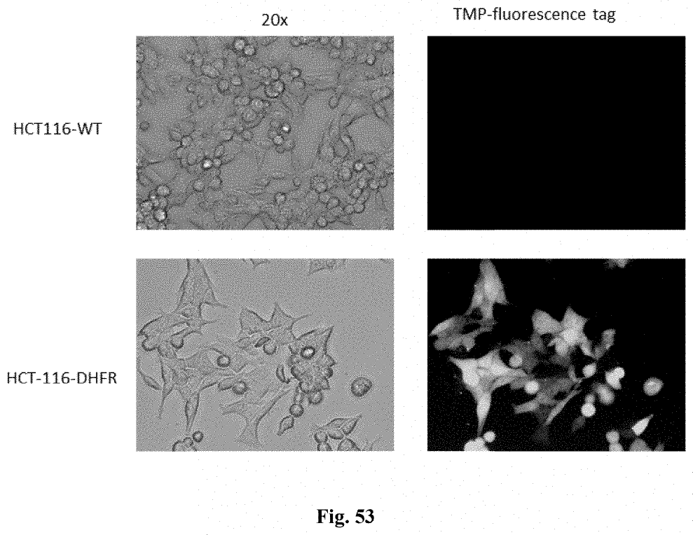

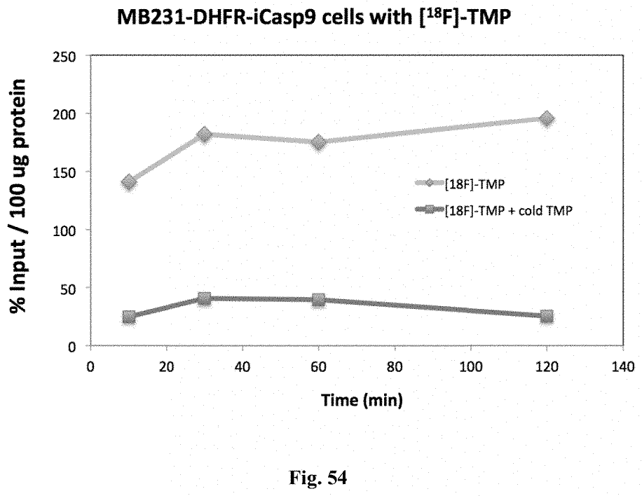

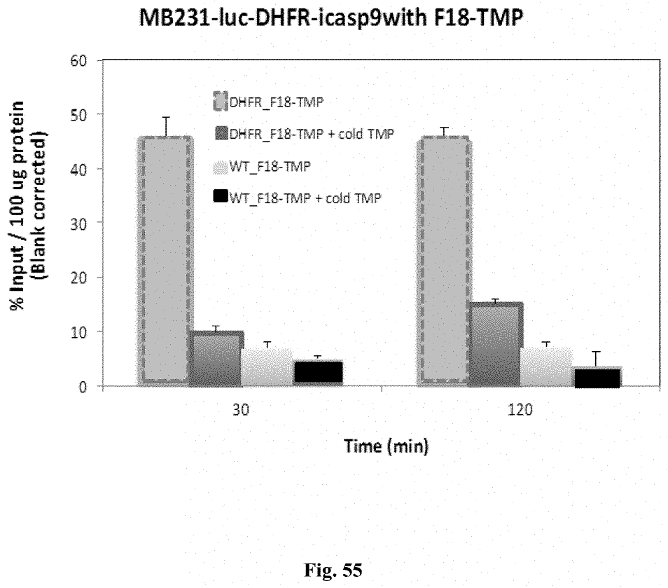

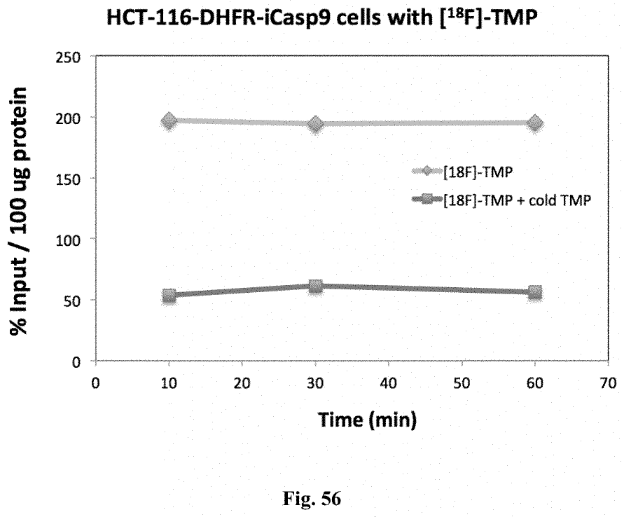

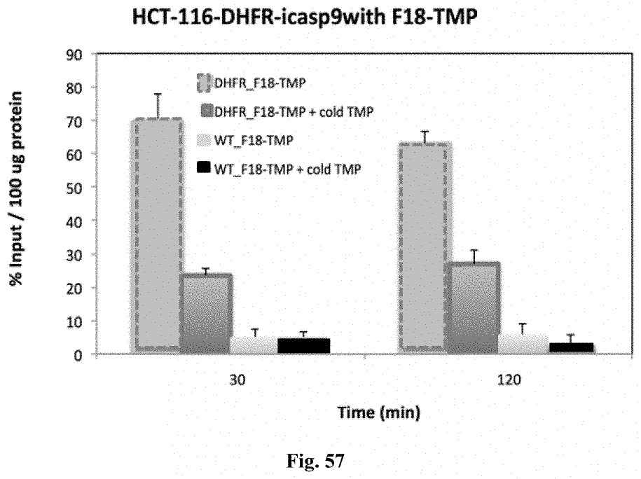

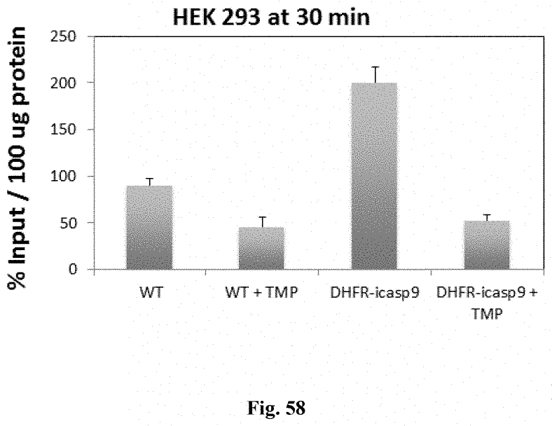

A western blot depicted the eDHFR-iCasp9 fusion protein in HEK293, HCT116, and MB231 cells (FIG. 50). HEK293, HCT116, and MB231 cells transduced with DHFR-iCasp9 were labeled with a Ligand-link (fluorescein linked with TMP); untransduced cells were not labeled with fluorescent TMP (FIGS. 13-15).

Cell uptake studies were performed with [¹⁸F]TMP in cells transduced with eDHFR iCasp9, in a similar manner as described for [¹¹C]Shld1. [¹⁸F]TMP was taken up rapidly by MB231, HCT116, and HEK293 cells transduced with eDHFR iCasp9 (FIGS. 16-20). The uptake could be blocked by a large excess of cold TMP, consistent with low nonspecific binding; untransduced cells were also used as a control.

### Example 30: Development of a Functional DHFR-iCasp9 Suicide Gene, and Synthesis of a Library of Bis-TMP Compounds with Varying Linker Lengths

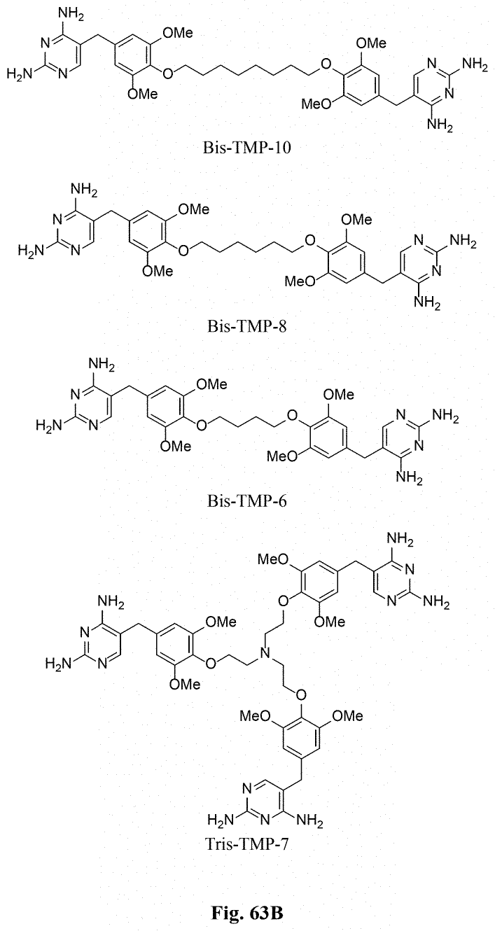

A library of Bis-TMP compounds was synthesized in which the linker between TMP molecules was 6, 8, 10, 16, 21, 27, or 33 atoms in length (FIGS. 63A-63B). A Tris-TMP was also synthesized (FIGS. 63A-63B).

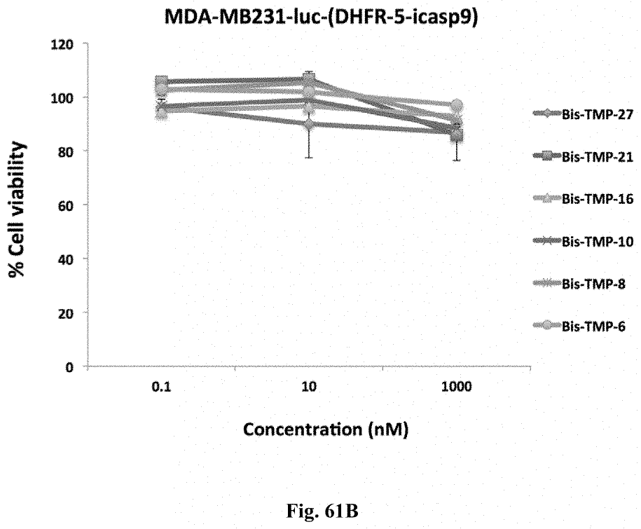

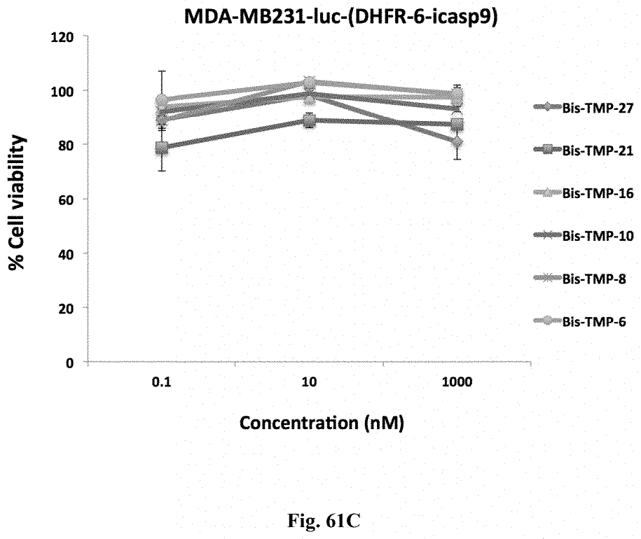

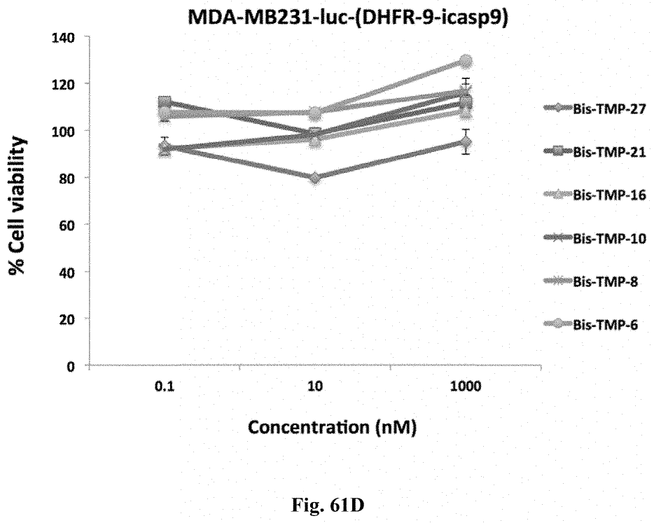

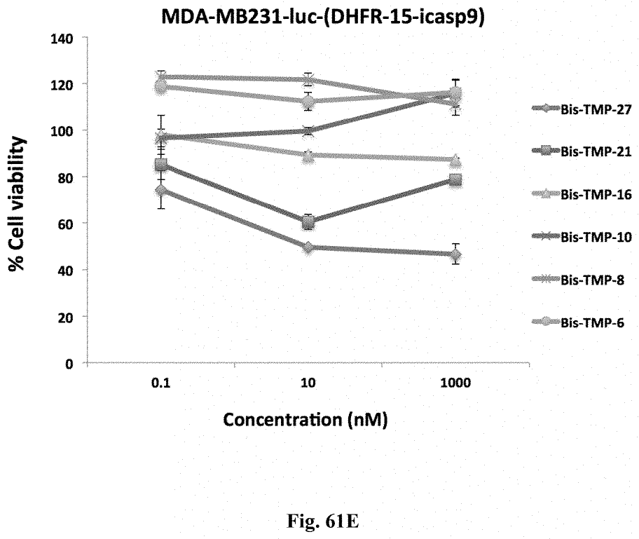

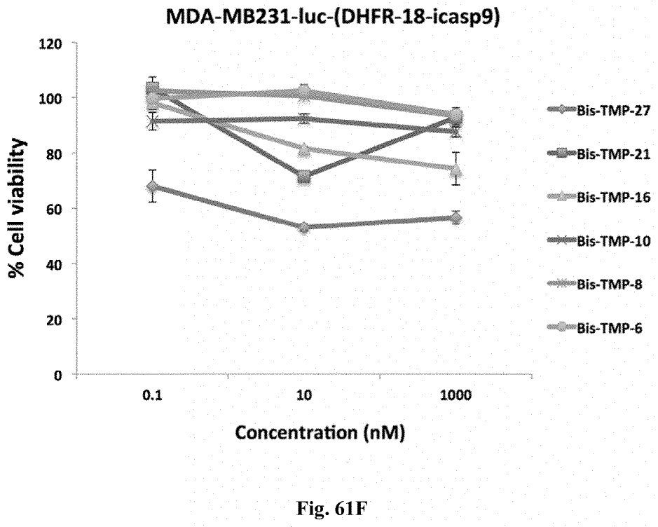

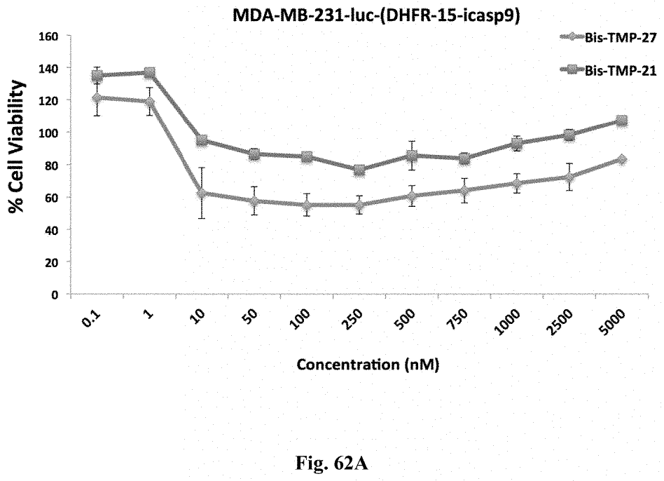

Various DHFR-iCasp9 constructs were created in which the linker between DHFR and iCasp9 was modified to be 5, 6, 9, 15, or 18 amino acids in length. The percentage viability of MDA-MB231 cells transduced with these DHFR-iCasp9 constructs was then assessed following treatment with a variety of Bis-TMP compounds at increasing concentrations (FIGS. 61A-61F). Wild-type MDA-MB231 cells were also evaluated as a control. Most combinations did not produce any cell killing; only DHFR-iCasp9 linkers of 15 or 18 amino acids, and Bis-TMP linkers of 21 and 27 atoms demonstrated cell killing.

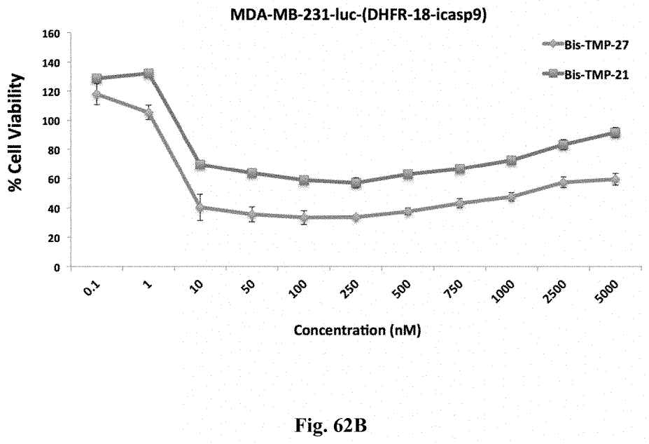

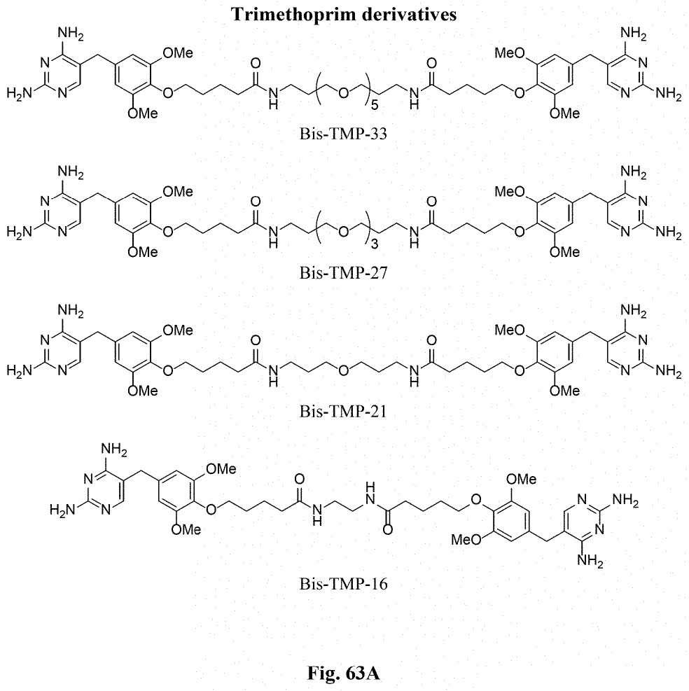

A more detailed analysis of the percentage (%) viability of MDA-MB231 cells transduced with DHFR-15-iCasp9 or DHFR-18-iCasp9 (with 15 or 18 amino acid linkers, respectively) was then assessed following treatment with Bis-TMP-21 or Bis-TMP-27 (with 21 or 27 atoms linkers, respectively) at increasing concentrations (FIGS. 62A-62B). These graphs illustrate potent activation of the DHFR-iCasp9 suicide gene at low nM concentrations of Bis-TMP. The DHFR-18-iCasp9 suicide gene and Bis-TMP-27 were the most effective combination, killing 70% of cells with an IC₅₀ of ˜5 nM (FIG. 62B).

### Example 31: Application

Further to the disclosure elsewhere herein, this disclosure includes the first dual function reporter gene, based on iCasp9, that serves as both a PET imaging reporter gene and a suicide gene. Although imaging reporter genes and potent suicide genes both exist, an imaging reporter gene (much less a dual function reporter gene) that is acceptable for routine clinical use has thus far not been contemplated. The disclosure fills a void in the clinical research toolbox by creating an imaging reporter gene that is acceptable for routine clinical use, in part because it has a potent safety switch. Since CAR T cells therapy and other gene therapies carry significant risk, the elegance of a dual function suicide-reporter is key from both a regulatory and patient perspective. Also, one aspect of this disclosure includes a modified iCasp9, in which the ligand binding domain has been replaced with eDHFR and is capable of being imaged with [¹⁸F]TMP.

The present disclosure includes a dual function reporter gene that has the potential to find widespread use in CAR T cell therapy and other cell-based therapies as both a PET imaging reporter gene and a suicide gene. The disclosure provides methods for biodistribution and patterns of trafficking of CAR T cells, provides insight into the mechanism of action of CAR T cell therapy, including an assessment of therapy-related toxicities, and helps in the design of more effective T cell therapies (and other cell-based therapies) for cancer. The imaging tools of this disclosure can be used routinely in the clinic to answer questions about response and therapy-related toxicities, and the presence of a potent suicide gene serves as a valuable safety switch which could be employed to control the many toxicities that are inherent in CAR T cell therapy (such as cytokine release syndrome, neurological toxicity, on-target/off-tumor toxicity, insertional oncogenesis, graft versus host disease, and off-target antigen recognition).

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

### REFERENCES

1. June, C. H. & Sadelain, M. Chimeric Antigen Receptor Therapy. N Engl J Med 379, 64-73(2018).
2. Pantel, A. R. & Mankoff, D. A. Molecular imaging to guide systemic cancer therapy: Illustrative examples of PET imaging cancer biomarkers. Cancer Lett 387, 25-31 (2017).
3. Weissleder, R., Schwaiger, M. C., Gambhir, S. S. & Hricak, H. Imaging approaches to optimize molecular therapies. Sci Transl Med 8, 355ps316 (2016).
4. Collins, S. A., Hiraoka, K., Inagaki, A., Kasahara, N. & Tangney, M. PET imaging for gene & cell therapy. Current gene therapy 12, 20-32 (2012).
5. Yaghoubi, S., et al. Human pharmacokinetic and dosimetry studies of [(18)F]FHBG: a reporter probe for imaging herpes simplex virus type-1 thymidine kinase reporter gene expression. J Nucl Med 42, 1225-1234 (2001).
6. Yaghoubi, S. S., et al. Noninvasive detection of therapeutic cytolytic T cells with 18F-FHBG PET in a patient with glioma. Nat Clin Pract Oncol 6, 53-58 (2009).
7. Krebs, S., et al. Antibody with Infinite Affinity for In Vivo Tracking of Genetically Engineered Lymphocytes. J Nucl Med 59, 1894-1900 (2018).
8. Castanares, M. A., et al. Evaluation of prostate-specific membrane antigen as an imaging reporter. J Nucl Med 55, 805-811 (2014).
9. Moroz, M. A., et al. Comparative Analysis of T Cell Imaging with Human Nuclear Reporter Genes. J Nucl Med 56, 1055-1060 (2015).
10. Traversari, C., et al. The potential immunogenicity of the TK suicide gene does not prevent full clinical benefit associated with the use of TK-transduced donor lymphocytes in HSCT for hematologic malignancies. Blood 109, 4708-4715 (2007).
11. Keu, K. V., et al. Reporter gene imaging of targeted T cell immunotherapy in recurrent glioma. Sci Transl Med 9(2017).
12. Klebanoff, C. A., Rosenberg, S. A. & Restifo, N. P. Prospects for gene-engineered T cell immunotherapy for solid cancers. Nat Med 22, 26-36 (2016).
13. Sellmyer, M. A., et al. Quantitative PET Reporter Gene Imaging with [11C]Trimethoprim. Mol Ther 25, 120-126 (2017).
14. Sellmyer, M. A., et al. Bacterial infection imaging with [18F]fluoropropyl-trimethoprim. Proc Natl Acad Sci USA 114, 8372-8377 (2017).
15. Richman, S. A., et al. High-Affinity GD2-Specific CAR T Cells Induce Fatal Encephalitis in a Preclinical Neuroblastoma Model. Cancer Immunol Res 6, 36-46 (2018).
16. Baccanari, D. P. & Kuyper, L. F. Basis of selectivity of antibacterial diaminopyrimidines. in J Chemother, Vol. 5 393-399 (1993).
17. Matthews, D. A., et al. Dihydrofolate reductase: x-ray structure of the binary complex with methotrexate. Science 197, 452-455 (1977).
18. Parker, K. C., Bednarek, M. A. & Coligan, J. E. Scheme for ranking potential HLA-A2 binding peptides based on independent binding of individual peptide side-chains. J Immunol 152, 163-175 (1994).
19. Ellebrecht, C. T., et al. Reengineering chimeric antigen receptor T cells for targeted therapy of autoimmune disease. Science 353, 179-184 (2016).
20. Bennett, J. Taking Stock of Retinal Gene Therapy: Looking Back and Moving Forward. Mol Ther 25, 1076-1094 (2017).
21. Calloway, N. T., et al. Optimized fluorescent trimethoprim derivatives for in vivo protein labeling. in Chembiochem, Vol. 8 767-774 (2007).
22. Bushby, S. R. & Hitchings, G. H. Trimethoprim, a sulphonamide potentiator. Br J Pharmacol Chemother 33, 72-90 (1968).
23. Singh, N., et al. Nature of tumor control by permanently and transiently modified GD2 chimeric antigen receptor T cells in xenograft models of neuroblastoma. Cancer Immunol Res 2, 1059-1070 (2014).
24. Weist, M. R., et al. PET of Adoptively Transferred Chimeric Antigen Receptor T Cells with (89)Zr-Oxine. J Nucl Med 59, 1531-1537 (2018).
25. Parente-Pereira, A. C., et al. Trafficking of CAR-engineered human T cells following regional or systemic adoptive transfer in SCID beige mice. J Clin Immunol 31, 710-718 (2011).
26. Liu, C. T., et al. Functional significance of evolving protein sequence in dihydrofolate reductase from bacteria to humans. Proc Natl Acad Sci USA 110, 10159-10164 (2013).
27. Campbell, D. O., et al. Structure-guided engineering of human thymidine kinase 2 as a positron emission tomography reporter gene for enhanced phosphorylation of non-natural thymidine analog reporter probe. J Biol Chem 287, 446-454 (2012).
28. Milone, M. C., et al. Chimeric receptors containing CD137 signal transduction domains mediate enhanced survival of T cells and increased antileukemic efficacy in vivo. Mol Ther 17, 1453-1464 (2009).
29. Oefner, C., et al. Increased hydrophobic interactions of iclaprim with Staphylococcus aureus dihydrofolate reductase are responsible for the increase in affinity and antibacterial activity. J Antimicrob Chemother 63, 687-698 (2009).

What is claimed is:

1. An engineered cell comprising a chimeric antigen receptor (CAR) and further comprising a nucleic acid molecule comprising a ligand binding domain capable of binding to a radiolabeled tracer.

2. The engineered cell of claim 1, wherein the cell is a T cell.

3. The engineered cell of claim 1, wherein the ligand binding domain is E. coli dihydrofolate reductase (eDHFR).

4. The engineered cell of claim 1, wherein the radiolabeled tracer is [¹⁸F]fluoropropyl-trimethoprim ([¹⁸F]FPTMP).

5. A method of assessing the efficacy or toxicity of an adoptive cell therapy in a subject, the method comprising:
a. administering to the subject an engineered T cell comprising a chimeric antigen receptor (CAR) and a nucleic acid molecule comprising a ligand binding domain;
b. administering to the subject a radiolabeled tracer capable of binding to the ligand binding domain;
c. detecting the amount of radiolabeled tracer bound by imaging; and,
d. assessing the efficacy or toxicity of the adoptive cell therapy in the subject.

6. A method of detecting the quantity of engineered T cells in a subject, the method comprising:
a. administering to the subject an engineered T cell comprising a chimeric antigen receptor (CAR) and a nucleic acid molecule comprising a ligand binding domain;
b. administering to the subject a radiolabeled tracer capable of binding to the ligand binding domain; and,
c. imaging the amount of radiolabeled tracer bound thereby detecting the quantity of engineered T cells in the subject.

7. A method of monitoring an immunotherapy treatment in a subject, the method comprising:
a. administering to the subject an engineered T cell comprising a chimeric antigen receptor (CAR) and a nucleic acid molecule comprising a ligand binding domain;
b. administering to the subject a radiolabeled tracer capable of binding to the ligand binding domain; and,
c. detecting the level of radiolabeled tracer bound by imaging as a measure of the immunotherapy treatment.

8. The method of claim 5, wherein the imaging is performed by positron emission tomography (PET), computerized tomography (CT) or bioluminescence (BL).

9. A method of imaging engineered T cells in a subject, the method comprising:
a. administering to the subject an engineered T cell comprising a chimeric antigen receptor (CAR) and a nucleic acid molecule comprising a ligand binding domain;
b. administering to the subject a radiolabeled tracer capable of binding to the ligand binding domain; and,
c. detecting the radiolabeled tracer by imaging using positron emission tomography (PET) or computed tomography (CT).

10. The method of claim 5, wherein the ligand binding domain is E. coli dihydrofolate reductase (eDHFR).

11. The method of claim 5, wherein the radiolabeled tracer is [¹⁸F]fluoropropyl-trimethoprim ([¹⁸F]FPTMP).

12. The method of claim 5, wherein the engineered T cell(s) is/are autologous to the subject.

13. The method of claim 5, wherein the engineered T cell(s) is/are allogenic to the subject.

14. The method of claim 5, wherein the subject is a mammal.

15. The method of claim 14, wherein the mammal is a human.


SEQUENCE LISTING (46 sequences)

SEQ ID NO: 1 - DNA, 2211 bp, Artificial Sequence
  Artificially synthesized
ggatccatgataagtttgattgctgctctggctgtggaccgggtaatcggtatggaaaacgccatgccctggaacctgcctgccgatttggcttggttcaagcgcaataccctgaacaaaccagtaatcatgggaaggcatacatgggaaagcattggaagaccacttcccggtagaaagaatattatcctgtctagccagcccggcacggatgatagggtgacatgggtaaagagcgtcgatgaggcgattgcggcgtgtggtgacgtgccggaaattatggttatcggaggcggcagggtctacgaacagttcctgccgaaggcacagaagctgtacctcacccacatcgatgcagaggtggaaggagacacgcactttccagattacgagcctgatgactgggagagtgtttttagcgaattccatgacgcagacgcccaaaactctcactcctactgctttgagattctcgaacgaagggcatgcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagggcagcggagagggcagaggaagtcttctaacatgcggtgacgtggaggagaatcccggccctgctagcacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataagtcgac

SEQ ID NO: 2 - protein, 732 aa, Artificial Sequence
  Artificially synthesized
MISLIAALAVDRVIGMENAMPWNLPADLAWFKRNTLNKPVIMGRHTWESIGRPLPGRKNIILSSQPGTDDRVTWVKSVDEAIAACGDVPEIMVIGGGRVYEQFLPKAQKLYLTHIDAEVEGDTHFPDYEPDDWESVFSEFHDADAQNSHSYCFEILERRACVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKGSGEGRGSLLTCGDVEENPGPASTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 3 - DNA, 8975 bp, Artificial Sequence
  Artificially synthesized
attgatcacgtgaggctccggtgcccgtcagtgggcagagcgcacatcgcccacagtccccgagaagttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagaggatccatgataagtttgattgctgctctggctgtggaccgggtaatcggtatggaaaacgccatgccctggaacctgcctgccgatttggcttggttcaagcgcaataccctgaacaaaccagtaatcatgggaaggcatacatgggaaagcattggaagaccacttcccggtagaaagaatattatcctgtctagccagcccggcacggatgatagggtgacatgggtaaagagcgtcgatgaggcgattgcggcgtgtggtgacgtgccggaaattatggttatcggaggcggcagggtctacgaacagttcctgccgaaggcacagaagctgtacctcacccacatcgatgcagaggtggaaggagacacgcactttccagattacgagcctgatgactgggagagtgtttttagcgaattccatgacgcagacgcccaaaactctcactcctactgctttgagattctcgaacgaagggcatgcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagggcagcggagagggcagaggaagtcttctaacatgcggtgacgtggaggagaatcccggccctgctagcacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacattaaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgc

SEQ ID NO: 4 - protein, 9 aa, Homo sapiens
ASETLTNIY

SEQ ID NO: 5 - protein, 9 aa, Homo sapiens
IAEIRDLAR

SEQ ID NO: 6 - protein, 9 aa, Homo sapiens
DPELPTLLR

SEQ ID NO: 7 - protein, 9 aa, Homo sapiens
LLAPNGDLY

SEQ ID NO: 8 - protein, 9 aa, Homo sapiens
LPEAEHADR

SEQ ID NO: 9 - protein, 9 aa, Homo sapiens
VPEPMTYWQ

SEQ ID NO: 10 - protein, 9 aa, Homo sapiens
RIEDTLFAL

SEQ ID NO: 11 - protein, 9 aa, Homo sapiens
RLDLAMLSA

SEQ ID NO: 12 - protein, 9 aa, Homo sapiens
PTDGRAASR

SEQ ID NO: 13 - protein, 9 aa, Homo sapiens
RGDPELPTL

SEQ ID NO: 14 - protein, 9 aa, Homo sapiens
ATDAVLAPH

SEQ ID NO: 15 - protein, 9 aa, Homo sapiens
VYVPEPMTY

SEQ ID NO: 16 - protein, 9 aa, Homo sapiens
RPDPEDGAG

SEQ ID NO: 17 - protein, 9 aa, Homo sapiens
MTPQAVLAF

SEQ ID NO: 18 - protein, 9 aa, Homo sapiens
LLCYPAARY

SEQ ID NO: 19 - protein, 9 aa, Homo sapiens
DLLANTVRY

SEQ ID NO: 20 - protein, 9 aa, Homo sapiens
EAEHADRLA

SEQ ID NO: 21 - protein, 9 aa, Homo sapiens
HPIASLLCY

SEQ ID NO: 22 - protein, 9 aa, Homo sapiens
NGDLYHIFA

SEQ ID NO: 23 - protein, 9 aa, Homo sapiens
YIDGPHGVG

SEQ ID NO: 24 - protein, 9 aa, Homo sapiens
EGDTHFPDY

SEQ ID NO: 25 - protein, 9 aa, Homo sapiens
FSEFHDADA

SEQ ID NO: 26 - protein, 9 aa, Homo sapiens
PADLAWFKR

SEQ ID NO: 27 - protein, 9 aa, Homo sapiens
SSQPGTDDR

SEQ ID NO: 28 - protein, 9 aa, Homo sapiens
EPDDWESVF

SEQ ID NO: 29 - protein, 9 aa, Homo sapiens
GTDDRVTWV

SEQ ID NO: 30 - protein, 9 aa, Homo sapiens
VPEIMVIGG

SEQ ID NO: 31 - protein, 9 aa, Homo sapiens
SVDEAIAAC

SEQ ID NO: 32 - protein, 9 aa, Homo sapiens
RVYEQFLPK

SEQ ID NO: 33 - protein, 9 aa, Homo sapiens
DAEVEGDTH

SEQ ID NO: 34 - protein, 9 aa, Homo sapiens
GMENAMPWN

SEQ ID NO: 35 - protein, 9 aa, Homo sapiens
CGDVPEIMV

SEQ ID NO: 36 - protein, 9 aa, Homo sapiens
DADAQNSHS

SEQ ID NO: 37 - protein, 9 aa, Homo sapiens
MVIGGGRVY

SEQ ID NO: 38 - protein, 9 aa, Homo sapiens
NAMPWNLPA

SEQ ID NO: 39 - protein, 9 aa, Homo sapiens
DWESVFSEF

SEQ ID NO: 40 - protein, 9 aa, Homo sapiens
DYEPDDWES

SEQ ID NO: 41 - protein, 9 aa, Homo sapiens
LIAALAVDR

SEQ ID NO: 42 - protein, 9 aa, Homo sapiens
NLPADLAWF

SEQ ID NO: 43 - protein, 9 aa, Homo sapiens
LNKPVIMGR

SEQ ID NO: 44 - protein, 376 aa, Herpes simplex virus
MASHAGQQHAPAFGQAARASGPTDGRAASRPSHRQGASEARGDPELPTLLRVYIDGPHGVGKTTTSAQLMEALGPRDNIVYVPEPMTYWQVLGASETLTNIYNTQHRLDRGEISAGEAAVVMTSAQITMSTPYAATDAVLAPHIGGEAVGPQAPPPALTLVFDRHPIASLLCYPAARYLMGNMTPQAVLAFVALMPPTAPGTNLVLGVLPEAEHADRLARRQRPGARLDLAMLSAIRRVYDLLANTVRYLQRGGRWREDWGRLTGVAAATPRPDPEDGAGSLPRIEDTLFALFRVPELLAPNGDLYHIFAWVLDVLADRLLPMHLFVLDYDQSPVGCRDALLRLTAGMIPTRVTTAGSIAEIRDLARTFAREVGGV

SEQ ID NO: 45 - protein, 159 aa, Escherichia coli [sequence omitted]

SEQ ID NO: 46 - protein, 5 aa, Artificial Sequence
  Artificially synthesized
SGGGS